(19) United States
(12) Patent Application Publication
(10) Pub. No.: US 2007/0036828 A1
(43) Pub. Date: Feb. 15, 2007

(54) Group b streptococcus vaccine

(75) Inventors: Rino Rappuoli, Castelnuovo Berardenga (IT); John Telford, Siena (IT); Guido Grandi, Siena (IT)

Correspondence Address: NOVARTIS VACCINES AND DIAGNOSTICS INC., CORPORATE INTELLECTUAL PROPERTY R338, P.O. BOX 8097, Emeryville, CA, 94662-8097, US

(73) Assignee: CHIRON CORPORATION, Emeryville, CA (US)

(21) Appl. No.: 10/527,672

(22) PCT Filed: Sep. 15, 2003
(86) PCT No.: PCT/US03/29167

§ 371 Date: Apr. 18, 2006

Related U.S. Application Data
(60) Provisional application No. 60/410,839, filed on Sep. 13, 2002.

(51) Int. Cl. A61K 39/08; C08B 37/00

(52) U.S. Cl. 424/239.1; 536/53

(57) ABSTRACT

This application relates to improved Group B Streptococcus (“GBS”) saccharide-based vaccines comprising combinations of GBS polysaccharides with polypeptide antigens, and vice versa, such that the polypeptide and the saccharide each contribute to the immunological response in a recipient. The combination is particularly advantageous where the saccharide and polypeptide are from different GBS serotypes. The combined antigens may be present as a simple combination where separate saccharide and polypeptide antigens are administered together, or they may be present as a conjugated combination, where the saccharide and polypeptide antigens are covalently linked to each other. Preferably, the immunogenic compositions of the invention comprise a GBS saccharide antigen and at least two GBS polypeptide antigens, wherein said GBS saccharide antigen comprises a saccharide selected form GBS serotype Ia, Ib, and III, and wherein said GBS polypeptide antigens comprise a combination of at least two polypeptide or fragments thereof selected from the antigen group consisting of GBS 80, GBS 91, GBS 104, GBS 147, GBS 173, GBS 276, GBS 305, GBS 313, GBS 322, GBS 328, GBS 330, GBS 338, GBS 358, GBS 361, GBS 404, GBS 656, GBS 690, and GBS 691.

This application claims the benefit of U.S. Provisional Patent Application Ser. No. 60/410,839, filed Sep. 13, 2002, which application is incorporated herein by reference in its entirety.

### TECHNICAL FIELD

This invention relates to polysaccharides from the bacteria Streptococcus agalactiae (GBS) and to their use in immunisation.

### BACKGROUND ART

Once thought to infect only cows, the Gram-positive bacterium Streptococcus agalactiae (or “group B streptococcus”, abbreviated to “GBS” (Ref 1) is now known to cause serious disease, bacteremia and meningitis, in immunocompromised individuals and in neonates. There are two types of neonatal infection. The first (early onset, usually within 5 days of birth) is manifested by bacteremia and pneumonia. It is contracted vertically as a baby passes through the birth canal. GBS colonises the vagina of about 25% of young women, and approximately 1% of infants born via a vaginal birth to colonised mothers will become infected. Mortality is between 50-70%. The second is a meningitis that occurs 10 to 60 days after birth. If pregnant women are vaccinated with type III capsule so that the infants are passively immunised, the incidence of the late onset meningitis is reduced but is not entirely eliminated.

The “B” in “GBS” refers to the Lancefield classification, which is based on the antigenicity of a carbohydrate which is soluble in dilute acid and called the C carbohydrate. Lancefield identified 13 types of C carbohydrate, designated A to O , that could be serologically differentiated. The organisms that most commonly infect humans are found in groups A, B, D, and G. Within group B, strains can be divided into at least 9 serotypes (Ia, Ib, Ia/c, II, III, IV, V, VI, VII and VIII) based on the structure of their polysaccharide capsule. In the past, serotypes Ia, Ib, II, and III were equally prevalent in normal vaginal carriage and early onset sepsis in newborns. Type V GBS has emerged as an important cause of GBS infection in the USA, however, and strains of types VI and VII have become prevalent among Japanese women.

The genome sequence of a serotype V strain 2603 V/R has been published (Ref 2) and various polypeptides for use a vaccine antigens have been identified (Ref 3). The vaccines currently in clinical trials, however, are based on polysaccharide antigens. These suffer from serotype-specificity and poor immunogenicity, and so there is a need for effective vaccines against S. agalactiae infection.

It is an object of the invention to provide further and improved GBS vaccines.

### DISCLOSURE OF THE INVENTION

The inventors have realised that saccharide-based vaccines can be improved by using them in combination with polypeptide antigens, and vice versa, such that the polypeptide and the saccharide each contribute to the immunological response in a recipient. The combination is particularly advantageous where the saccharide and polypeptide are from different GBS serotypes.

The combined antigens may be present as a simple combination where separate saccharide and polypeptide antigens are administered together, or they may be present as a conjugated combination, where the saccharide and polypeptide antigens are covalently linked to each other.

Thus the invention provides an immunogenic composition comprising (i) one or more GBS polypeptide antigens and (ii) one or more GBS saccharide antigens. The polypeptide and the polysaccharide may advantageously be covalently linked to each other to form a conjugate.

Between them, the combined polypeptide and saccharide antigens preferably cover two or more GBS serotypes (e.g. 2, 3, 4, 5, 6, 7, 8 or more serotypes). The serotypes of the polypeptide and saccharide antigens may or may not overlap. For example, the polypeptide might protect against serogroup II or V, while the saccharide protects against either serogroups Ia, Ib, or III. Preferred combinations protect against the following groups of serotypes: (1) serotypes Ia and Ib, (2) serotypes Ia and II, (3) serotypes Ia and III, (4) serotypes Ia and IV, (5) serotypes Ia and V, (6) serotypes Ia and VI, (7) serotypes Ia and VII, (8) serotypes Ia and VIII, (9) serotypes Ib and II, (10) serotypes Ib and III, (11) serotypes Ib and IV, (12) serotypes Ib and V, (13) serotypes Ib and VI, (14) serotypes Ib and VII, (15) serotypes Ib and VIII, 16) serotypes II and III, (17) serotypes II and IV, (18) serotypes II and V, (19) serotypes II and VI, (20) serotypes II and VII, (21) serotypes II and VII, (22) serotypes III and IV, (23) serotypes III and V, (24) serotypes III and VI (25) serotypes III and VII, (26) serotypes III and VIII, (27) serotypes IV and V, (28) serotypes IV and VI, (29) serotypes IV and VII, (30) serotypes IV and VIII, (31) serotypes V and VI, (32) serotypes V and VII, (33) serotypes V and VIII, (34) serotypes VI and VII, (35) serotypes VI and VIII, and (36) serotypes VII and VIII.

Still more preferably, the combinations protect against the following groups of serotypes: (1) serotypes Ia and II, (2) serotypes Ia and V, (3) serotypes Ib and II, (4) serotypes Ib and V, (5) serotypes III and II, and (6) serotypes III and V. Most preferably, the combinations protect against serotypes III and V.

Protection against serotypes II and V is preferably provided by polypeptide antigens. Protection against serotypes Ia, Ib and/or III may be polypeptide or saccharide antigens.

Preferably, the immunogenic composition comprises one or more serogroup V antigens or fragments thereof selected from the antigen group consisting of GBS 80, GBS 91, GBS 104, GBS 147, GBS 173, GBS 276, GBS 305, GBS 313, GBS 322, GBS 328, GBS 330, GBS 338, GBS 358, GBS 361, GBS 404, GBS 656, GBS 690, and GBS 691. Preferably, the composition comprises a composition of at least two of these GBS antigens or a fragment thereof.

In one embodiment, the immunogenic composition comprises a GBS saccharide antigen and at least two GBS polypeptide antigens or fragments thereof, wherein said GBS saccharide antigen comprises a saccharide selected from GBS serotype Ia, Ib, and III, and wherein said GBS polypeptide antigens comprise a combination of at least two polypeptide or a fragment thereof selected from the antigen group consisting of GBS 80, GBS 91, GBS 104, GBS 147, GBS 173, GBS 276, GBS 305, GBS 313, GBS 322, GBS 328, GBS 330, GBS 338, GBS 358, GBS 361, GBS 404, GBS 656, GBS 690, and GBS 691.

Preferably, the combination comprises GBS 80 or a fragment thereof. In one embodiment, the GBS polypeptide antigens comprise a combination of two GBS antigens or fragments thereof selected from the antigen group consisting of (1) GBS 80 and GBS 91, (2) GBS 80 and GBS 104, (3) GBS 80 and GBS 147, (4) GBS 80 and GBS 173, (5) GBS 80 and GBS 276, (6) GBS 80 and GBS 305, (7) GBS 80 and GBS 313, (8) GBS 80 and GBS 322, (9) GBS 80 and GBS 328, (10) GBS 80 and GBS 330, (11) GBS 80 and GBS 338, (12) GBS 80 and GBS 358, (13) GBS 80 and GBS 361, (14) GBS 80 and GBS 404, (14) GBS 80 and GBS 404, (15) GBS 80 and GBS 656, (16) GBS 80 and GBS 690, and (17) GBS 80 and GBS 691.

Still more preferably, the combination is selected from the antigen group consisting of (1) GBS 80 and GBS 338; (2) GBS 80 and GBS 361, (3) GBS 80 and GBS 305, (4) GBS 80 and GBS 328, (5) GBS 80 and GBS 690, (6) GBS 80 and GBS 691 and (7) GBS 80 and GBS 147. Even more preferably, the combination comprises GBS 80 and GBS 691.

In one embodiment, the composition comprises a combination at least three GBS polypeptide antigens. Preferably, this combination comprises GBS 80 and GBS 691.

Preferably, the immunogenic composition further comprises a GBS polypeptide or a fragment thereof of serogroup II.

The Polypeptide Antigen

The polypeptide is preferably: (a) a polypeptide comprising an amino acid sequence selected from the group consisting of the even-numbered SEQ IDs 2-10966 from Ref. 3; (b) a polypeptide comprising an amino acid sequence having sequence identity to an amino acid sequence from in (a); or (c) a polypeptide comprising a fragment of an amino acid sequence from (a).

Within (a), preferred SEQ IDs are those which encode GBS1 to GBS689 (see Table IV of reference 3).

Within (b), the degree of sequence identity may vary depending on the amino acid sequence (a) in question, but is preferably greater than 50% (e.g. 60%, 70%, 80%, 90%, 95%, 99% or more). Polypeptides within (b) include homologs, orthologs, allelic variants and functional mutants of (a). Typically, 50% identity or more between two proteins is considered to be an indication of functional equivalence. Identity between proteins is preferably determined by the Smith-Waterman homology search algorithm as implemented in the MPSRCH program (Oxford Molecular), using an affine gap search with parameters gap open penalty=12 and gap extension penalty=1.

Within (c), the length of the fragment may vary depending on the amino acid sequence (a) in question, but the fragment is preferably at least 7 consecutive amino, acids from the sequences of (a) e.g. 8, 10, 12, 14, 16, 18, 20, 30, 40, 50, 60, 70, 80, 90, 100, 150, 200 or more. Preferably the fragment comprises one or more epitopes from the sequence. Other preferred fragments are the N-terminal signal peptides of SEQ IDs 1-10966 from Ref. 3, SEQ IDs 1-10966 from Ref 3 without their N-terminal signal peptides, and SEQ IDs 1-10966 from Ref 3 wherein up to 10 amino acid residues (i.e. 1, 2, 3, 4, 5, 6, 7, 8, 9 or 10 residues) are deleted from the N-terminus and/or the C-terminus e.g. the N-terminal amino acid residue may be deleted.

The polypeptides can, of course, be prepared by various means (e.g. recombinant expression, purification from GBS, chemical synthesis etc.) and in various forms (e.g. native, fusions, glycosylated, non-glycosylated etc.). They are preferably prepared in substantially pure form (i.e. substantially free from other streptococcal or host cell proteins) or substantially isolated form.

Preferred polypeptide antigens are: GBS 80, GBS 91, GBS 104, GBS 147, GBS 173, GBS 276, GBS 305, GBS 313, GBS 322, GBS 328, GBS 330, GBS 338, GBS 358, GBS 361, GBS 404, GBS 656, GBS 690, and GBS 691, including polypeptides having amino acid sequences with sequence identity thereto etc.

The nucleotide and amino acid sequences of GBS80 in Ref 3 are SEQ ID 8779 and SEQ ID 8780. These sequences are set forth below as SEQ ID NOS 1 and 2:

SEQ ID NO.1

ATGAAATTATCGAAGAAGTTATTGTTTTCGGCTGCTGTTTTAACAATGGT
GGCGGGGTCAACTGTTGAACCAGTAGCTCAGTTTGCGACTGGATGAGTAT
TGTAAGAGCTGCAGAAGTGTCACAAGAACGCCCAGCGAAACAACAGTAAA
TATCTATAAATTACAAGCTGATAGTTATAAATCGGAAATTACTTCTAATG
GTGGTATCGAGAATAAAGACGGCGAAGTAATATCTAACTATGCTAAACTT
GGTGACAATGTAAAAGGTTTGCAAGGTGTACAGTTTAAACGTTATAAAGT
CAAGACGGATATTTCTGTTGATGAATTGAAAAAATTGACAACAGTTGAAG
CAGCAGATGCAAAAGTGGAACGATTCTTGAAGAAGGTGTCAGTCTACCTC
AAAAAACTAATGCTCAAGGTTTGGTCGTCGATGCTCTGGATTCAAAAAGT
AATGTGAGATACTTGTATGTAGAAGATTTAAAGAATTCACCTTCAAACAT
TACCAAAGCTTATGCTGTACCGTTTGTGTTGGAATTACCAGTTGCTAACT
CTACAGGTACAGGTTTCCTTTCTGAAATTAATATTTACCCTAAAAACGTT
GTAACTGATGAACCAAAAACAGATAAAGATGTTAAAAAATTAGGTCAGGA
CGATGCAGGTTATACGATTGGTGAAGAATTCAAATGGTTCTTGAAATCTA
CAATCCCTGCCAATTTAGGTGACTATGAAAAATTTGAAATTACTGATAAA
TTTGCAGATGGCTTGACTTATAAATCTGTTGGAAAAATCAAGATTGGTTC
GAAAACACTGAATAGAGATGAGCACTACACTATTGATGAACCAACAGTTG
ATAACCAAAATACATTAAAAATTACGTTTAAACCAGAGAAATTTAAAGAA
ATTGCTGAGCTACTTAAAGGAATGACCCTTGTTAAAAATCAAGATGCTCT
TGATAAAGCTACTGCAAATACAGATGATGCGGCATTTTTGGAAATTCCAG
TTGCATCAACTATTAATGAAAAAGCAGTTTTAGGAAAAGCAATTGAAAAT
ACTTTTGAACTTCAATATGACCATACTCCTGATAAAGCTGACAATCCAAA
ACCATCTAATCCTCCAAGAAAACCAGAAGTTCATACTGGTGGGAAACGAT
TTGTAAAGAAAGACTCAACAGAAACACAAACACTAGGTGGTGCTGAGTTT
GATTTGTTGGCTTCTGATGGGACAGCAGTAAAATGGACAGATGCTCTTAT
TAAAGCGAATACTAATAAAAACTATATTGCTGGAGAAGCTGTTACTGGGC
AACCAATCAAATTGAAATCACATACAGACGGTACGTTTGAGATTAAAGGT
TTGGCTTATGCAGTTGATGCGAATGCAGAGGGTACAGCAGTAACTTACAA
ATTAAAAGAAACAAAAGCACCAGAAGGTTATGTAATCCCTGATAAAGAAA
TCGAGTTTACAGTATCACAAACATCTTATAATACAAAACCAACTGACATC
ACGGTTGATAGTGCTGATGCAACACCTGATACAATTAAAAACAACAAACG
TCCTTCAATCCCTAATACTGGTGGTATTGGTACGGCTATCTTTGTCGCTA
TCGGTGCTGCGGTGATGGCTTTTGCTGTTAAGGGGATGAAGCGTCGTACA
AAAGATAAC

SEQ ID NO:2

MKLSKKLLFSAAVLTMVAGSTVEPVAQFATGMSIVRAAEVSQERPAKTTV
NIYLQADSYKSEITSNGGIENKDGEVISNYAKLGDNVKGLQGVQFKRYKV
KTDISVDELKLTTVEAADAKVGTILEEGVSLPQKTNAQGLVVDALDSKSN
VRYLYVEDLKNSPSNITKAYAVPFVLELPVANSTGTGFLSEINIYPKNVV
TDEPKTDKDVKKLGQDDAGYTIGEEFKWFLKSTIPANLGDYEKFEITDKF
ADGLTYKSVGKIKIGSKTLNRDEHYTIDEPTVDNQNTLKITFKPEKFKEI
AELLKGMTLVKNQDALDKATANTDDAAFLEIPVASTINEKAVLGKAIENT
FELQYDHTPDKADNPKPSNPPRKPEVHTGGKRPVKKDSTETQTLGGAEFD
LLASDGTAVKWTDALIKANTNKNYIAGEAVTGQPIKLKSHTDGTFEIKGL
AYAVDANAEGTAVTYKLKETKAPEGYVIPDKEIEFTVSQTSYNTKPTDIT
VDSADATPDTIKNNKRPSIPNTGGIGTAIFVAIGAAVMAFAVKGMKRRTK
DN

The nucleotide and amino acid sequences of GBS 91 in Ref. 3 are SEQ ID 8937 and SEQ ID 8938. These sequences are set forth below as SEQ ID NOS 3 and 4:

SEQ ID NO.3

ATGAAAAAAGGACAAGTAAATGATACTAAGCAATCTTACTCTCTACGTAA
ATATAAATTTGGTTTAGCATCAGTAATTTTAGGGTCATTCATAATGGTCA
CAAGTCCTGTTTTTGCGGATCAAACTACATCGGTCAAGTTAATAATCAGA
CAGGCACTAGTGTGGATGCTAATAATTCTTCCAATGAGACAAGTGCGTCA
AGTGTGATTACTTCCAATAATGATAGTGTTCAAGCGTCTGATAAAGTTGT
AAATAGTCAAAATACGGCAACAAAGGACATTACTACTCCTTTAGTAGAGA
CAAAGCCAATGGTGGAAAAAACATTACCTGAACAAGGGAATTATGTTTAT
AGCAAAGAAACCGAGGTGAAAAATACACCTTCAAAATCAGCCCCAGTAGC
TTTCTATGCAAAGAAAGGTGATAAAGTTTTCTATGACCAAGTATTTAATA
AAGATAATGTGAAATGGATTTCATATAAGTCTTTTTGTGGCGTACGTCGA
TACGCAGTATTGAGTCACTAGATCCATCAGGAGGTTCAGAGACTAAAGCA
CCTACTCCTGTAACAAATTCAGGAAGCAATAATCAAGAGAAAATAGCAAC
GCAAGGAAATTATACATTTTCACATAAAGTAGAAGTAAAAAATGAAGCTA
AGGTAGCGAGTCCAACTCAATTTACATTGGACAAAGGAGACAGAATTTTT
TACGACCAAATACTAACTATTGAAGGAAATCAGTGGTTATCTTATAAATC
ATTCAATGGTGTTCGTCGTTTTGTTTTGCTAGGTAAAGCATCTTCAGTAG
AAAAAACTGAAGATAAAGAAAAAGTGTCTCCTCAACCACAAGCCCGTATT
ACTAAAACTGGTAGACTGACTATTTCTAACGAAACAACTACAGGTTTTGA
TATTTTAATTACGAATATTAAAGATGATAACGGTATCGCTGCTGTTAAGG
TACCGGTTTGGACTGAACAAGGAGGGCAAGATGATATTAAATGGTATACA
GCTGTAACTACGGGGATGGCAACTACAAAGTAGCTGTATCATTTGCTGAC
CATAAGAATGAGAAGGGTCTTTATAATATTCATTTATACTACCAAGAAGC
TAGTGGGACACTTGTAGGTGTAACAGGAACTAAAGTGACAGTAGCTGGAA
CTAATTCTTCTCAAGAACCTATTGAAAATGGTTTAGCAAAGACTGGTGTT
TATAATATTATCGGAAGTACTGAAGTAAAAAATGAAGCTAAAATATCAAG
TCAGACCCAATTTACTTTAGAAAAAGGTGACAAAATAAATTATGATCAAG
TATTGACAGCAGATGGTTACCAGTGGATTTCTTACAAATCTTATAGTGGT
GTTCGTCGCTATATTCCTGTGAAAAAGCTAACTACAAGTAGTGAAAAAGC
GAAAGATGAGGCGACTAAACCGACTAGTTATCCCAACTTACCTAAAACAG
GTACCTATACATTTACTAAAACTGTAGATGTGAAAAGTCAACCTAAAGTA
TCAAGTCCAGTGGAATTTAATTTTCAAAAGGGTGAAAAAATACATTATGA
TCAAGTGTTAGTAGTAGATGGTCATCAGTGGATTTCATACAAGAGTTATT
CCGGTATTCGTCGCTATATTGAAATT

SEQ ID NO.4

MKKGQVNDTKQSYSLRKYKFGLASVILGSFIMVTSPVFADQTTSVQVNNQ
TGTSVDANNSSNETSASSVITSNNDSVQASDKVVNSQNTATKDITTPLVE
TKPMVEKTLPEQGNYVYSKETEVKNTPSKSAPVAFYAKKGDKVFYDQVFN
KDNVKWISYKSFCGVRRYAAIESLDPSGGSETKAPTPVTNSGSNNQEKIA
TQGNYTFSHEVKNEAKVASPTQFTLDKGDRIFYDQILTIEGNQWLSYKSF
NGVRRFVLLGKASSVEKTEDKEKVSPQPQARITKTGRLTISNETTTGFDI
LITNIKDDNGIAAVKVPVWTEQGGQDDIKWYTAVTTGDGNYKVAVSFADH
NEKGLYNIHLYYQEASGTLVGTGTKVTVAGTNSSQEPIENGLAKTGVYNI
IGSTEVKISSQTQFTLEKGDKINYDQVLTADGYQWISYKSYSGVRRYIPV
KKLTTSSEKAKDEATKPTSYPNLPKTGTYTFTKTVDVKSQPKVSSPVEFN
FQKGEKIHYDQVLVVDGHQWISYKSYSGIRRYIEI

The nucleotide and amino acid sequences of GBS 104 in Ref. 3 are SEQ ID 8777 and SEQ ID 8778. These sequences are set forth below as SEQ ID NOS 5 and 6:

SEQ ID NO.5

ATGAAAAAGAGACAAAAAATATGGAGAGGGTTATCAGTTACTTTACTAAT
CCTGTCCCAAATTCCATTTGGTATATTGGTACAAGGTGAAACCCAAGATA
CCAATCAAGCACTTGGAAAAGTAATTGTTAAAAAAACGGGAGACAATGCT
ACACCATTAGGCAAAGCGACTTTTGTGTTAAAAAATGACAATGATAAGTC
AGAAACAAGTCACGAAACGGTAGAGGGTTCTGGAGAAGCAACCTTTGAAA
ACATAAAACCTGGAGACTACACATTAAGAGAAGAAACAGCACCAATTGGT
TATAAAAAAACTGATAAAACCTGGAAAGTTAAAGTTGCAGATAACGGAGC
AACAATAATCGAGGGTATGGATGCAGATAAAGCAGAGAAACGAAAAGAAG
TTTTGAATGCCCAATATCCAAAATCAGCTATTTATGAGGATACAAAAGAA
AATTACCCATTAGTTAATGTAGAGGGTTCCAAAGTTGGTGAACAATACAA
AGCATTGAATCCAATAAATGGAAAAGATGGTCGAAGAGAGATTGCTGAAG
GTTGGTTATCAAAAAAAATTACAGGGGTCAATGATCTCGATAAGAATAAA
TATAAAATTGAATTAACTGTTGAGGGTAAAACCACTGTTGAAACGAAAGA
ACTTAATCAACCACTAGATGTCGTTGTGCTATTAGATAATTCAAATAGTA
TGAATAATGAAAGAGCCAATAATTCTCAAAGAGCATTAAAAGCTGGGGAA
GCAGTTGAAAAGCTGATTGATAAAATTACATCAAATAAAGACAATAGAGT
AGCTCTTGTGACATATGCCTCAACCATTTTTGATGGTACTGAAGCGACCG
TATCAAAGGGAGTTGCCGATCAAAATGGTAAAGCGCTGAATGATAGTGTA
TCATGGGATTATCATAAAACTACTTTTACAGCAACTACACATAATTACAG
TTATTTAAATTTAACAAATGATGCTAACGAAGTTAATATTCTAAAGTCAA
GAATTCCAAAGGAAGCGGAGCATATAAATGGGGATCGCACGCTCTATCAA
TTTGGTGCGACATTTACTCAAAAAGCTCTAATGAAAGCAAATGAAATTTT
AGAGACACAAAGTTCTAATGCTAGAAAAAAACTTATTTTTCACGTAACTG
ATGGTGTCCCTACGATGTCTTATGCCATAAATTTTAATCCTTATATATCA
ACATCTTACCAAAACCAGTTTAATTCTTTTTTAAATAAAATACCAGATAG
AAGTGGTATTCTCCAAGAGGATTTTATAATCAATGGTGATGATTATCAAA
TAGTAAAAGGAGATGGAGAGAGTTTTAAACTGTTTTCGGATAGAAAAGTT
CCTGTTACTGGAGGAACGACACAAGCAGCTTATCGAGTACCGCAAAATCA
ACTCTCTGTAATGAGTAATGAGGGATATGCAATTAATAGTGGATATATTT
ATCTCTATTGGAGAGATTACAACTGGGTCTATCCATTTGATCCTAAGACA
AAGAAAGTTTCTGCAACGAAACAAATCAAAACTCATGGTGAGCCAACAAC
ATTATACTTTAATGGAAATATAAGACCTAAAGGTTATGACATTTTTACTG
TTGGGATTGGTGTAAACGGAGATCCTGGTGCAACTCCTCTTGAAGCTGAG
AAATTTATGCAATCAATATCAAGTAAAACAGAAAATTATACTAATGTTGA
TGATACAAATAAAATTTATGATGAGCTAAATAAATACTTTAAAACAATTG
TTGAGGAAAAACATTCTATTGTTGATGGAAATGTGACTGATCCTATGGGA
GAGATGATTGAATTCCAATTAAAAAATGGTCAAAGTTTTACACATGATGA
TTACGTTTTGGTTGGAAATGATGGCAGTCAATTAAAAAATGGTGTGGCTC
TTGGTGGACCAAACAGTGATGGGGGAATTTTAAAAGATGTTACAGTGACT
TATGATAACATCTCAAACCATCAAAATCAATCATTTGAACTTAGGAAGTG
GACAAAAAGTAGTTCTTACCTATGATGTACGTTTAAAAGATAACTATATA
AGTAACAAATTTTACAATACAAATAATCGTACAACGCTAAGTCCGAAGAG
TGAAAAAGAACCAAATACTATTCGTGATTTCCCAATTCCCAAAATTCGTG
ATGTTCGTGAGTTTCCGGTACTAACCATCAGTAATCAGAAGAAAATGGGT
GAGGTTGAATTTATTAAAGTTAATAAAGACAAACATTCAGAATCGCTTTT
GGGAGCTAAGTTTCAACTTCAGATAGAAAAAGATTTTTCTGGGTATAAGC
AATTTGTTCCAGAGGGAAGTGATGTTACAACAAAGAATGATGGTAAAATT
TATTTTAAAGCACTTCAAGATGGTAACTATAAATTATATGAAATTTCAAG
TCCAGATGGCTATATAGAGGTTAAAACGAAACCTGTTGTGACATTTACAA
TTCAAAATGGAGAAGTTACGAACCTGAAAGCAGATCCAAATGCTAATAAA
AATCAAATCGGGTATCTTGAAGGAAATGGTAAACATCTTATTACCAACAC
TCCCAAACGCCCACCAGGTGTTTTTCCTAAAACAGGGGGAATTGGTACAA
TTGGTACAATTGTCTATATATTAGTTGGTTCTACTTTTATGATACTTACC
ATTTGTTCTTTCCGTCGTAAACAATTG

SEQ ID NO.6

MKKRQKIWRGLSVTLLILSQIPFGILVQGETQDTNQALGVIVKKTGDNAT
PLGKATFVLKNDNDKSETSHETVEGSGEATGENIKPGDYTLREETAPIGY
KKTDKTWKVKVADNGATIIEGMDADKAEKRKEVLNAQYPKSAIYEDTKEN
YPLVNVEGSKVGEQYKALNPINGKDGRREIAEGWLSKKITGVNDLDKNKY
KIELTVEGKTTVETKELNQPLDVVVLLDNSNSMNNERANNSQRALKAGEA
VEKLIDKITSNKDNRVALVTYASTIFDGTEATVSKGVADQNGKALNDSVS
WDYHKTTFTATTHNYSYLNLTNDANEVNILKSRIPKEAEHINGDRTLYQF
GATFQKALMKANEILETQSSNARKKLIFHVTDGVPTMSYAINFNPYISTS
YQNQFNSFLNKIPDRSGILQEDFIINGDDYQIVKGDGESFKLFSDRKVPV
TGGTTQAAYRVPQNQLSVMSNEGYAINSGYIYLYWRDYNWVYPFDKTKKV
SATKQIKTHGEPTTLYFNGNIRPKGYDIFTVGIGVNGDPGATPLEAEKFM
QSISSKTENYTNVDDTNKIYDELNKYFKTIVEEKHSIVDGNVTDPMGEMI
EFQLKNGQSFTHDDYVLVGNDGSQLKNGVALGGPNSDGGILKDVTVTYDK
TSQTIKINHLNLGSGQKVVLTYDVRLKDNYISNKFYNTNNRTTLSPKSEK
EPNTIRDFPIPKIRDVREFPVLTISNQKKMGEVEFIKVNKDKHSESLLGA
KFQLQIEKDFSGYKQFVPEGSDVTTKNDGKIYFKALQDGNYKLYEISSPD
GYIEVKTKPVVTFTIQNGEVTNLKADPNANKNQIGLEGNGKHLITNTPKR
PPGVFPKTGGIGTIVYILVGSTFMILTICSFRRKQL

The nucleotide and amino acid sequences of GBS 147 in Ref 3 are SEQ ID 8525 and SEQ ID 8526. These sequences are set forth below as SEQ ID NOS 7 and 8:

SEQ ID NO.7

GTGGATAAACATCACTCAAAAAGGCTATTTTAAAGTTAACACTTATAACA
ACTAGTATTTTATTAATGCATAGCAATCAAGTGAATGCAGAGGAGCAAGA
ATTAAAAAACCAAGAGCAATCACCTGTAATTGCTAATGTTGCTCAACAGC
CATCGCCATCGGTAACTACTAATACTGTTGAAAAAACATCTGTAACAGCT
GCTTCTGCTAGTAATACAGCGAAAGAAATGGGTGATACATCTGTAAAAAA
TGACAAAACAGAAGATGAATTATTAGAAGAGTTATCTAAAAACCTTGATA
CGTCTAATTTGGGGGCTGATCTTGAAGAAGAATATCCCTCTAAACCAGAG
ACAACCAACAATAAAGAAAGCAATGTAGTAACAAATGCTTCAACTGCAAT
AGCACAGAAAGTTCCCTCAGCATATGAAGAGGTGAAGCCAGAAAGCAAGT
CATCGCTTGCTGTTCTTGATACATCTAAAATAACAAAATTACAAGCCATA
ACCCAAAGAGGAAATGTAGTAGCTATTATTGATACTGGCTTTGATATTAA
CCATGATATTTTTCGTTTAGATAGCCCAAAAGATGATAAGCACAGCTTTT
AAAACTAAGACAGAATTTGAGGAATTAAAAGCAAAACATAATATCACTTA
TGGGAAATGGGTTAACGATAAGATTGTTTTTGCACATAACTACGCCAACA
ATACAGAAACGGTGGCTGATATTGCAGCAGCTATGAAAGATGGTTATGGT
TCAGAAGCAAAGAATATTTCGCATGGTACACACGTTGCTGGTATTTTTGT
AGGTAATAGTAAACGTCCAGCAATCAATGGTCTTCTTTTAGAAGGTGCAG
CGCCAAATGCTCAAGTCTTATTAATGCGTATTCCAGATAAAATTGATTCG
GACAAATTTGGTGAAGCATATGCTAAAGCAATCACAGACGCTGTTAATCT
AGGAGCAAAAACGATTAATATGAGTATTGGAAAAACAGCTGATTCTTTAA
TTGCTCTCAATGATAAAGTTAAATTAGCACTTAAATTAGCTTCTGAGAAG
GGCGTTGCAGTTGTTGTGGCTGCCGGAAATGAAGGCGCATTTGGTATGGA
TTATCAACTAATCCTGACTACGGTACGGTTAATAGTCCAGCTATTTCTGA
AGATACTTTGAGTGTTGCTAGCTATGAATCACTTAAAACTATCAGTGAGG
TCGTTGAAACAACTATTGAAGGTAAGTTAGTTAAGTTGCCGATTGTGACT
TCTAAACCTTTTGACAAAGGTAAGGCCTACGATGTGGTTTATGCCAATTA
TGGTGCAAAAAAAGACTTTGAAGGTAAGGACTTTAAAGGTAAGATTGCAT
TAATTGAGCGTGGTGGTGGACTTGATTTTATGACTAAAATCACTCATGCT
ACAAATGCAGGTGTTGTTGGTATCGTTATTTTTAACGATCAAGAAAAACG
TGGAAATTTTCTAATTCCTTACCGTGAATTACCTGTGGGGATTATTAGTA
AAGTAGATGGCGAGCGTATAAAAAATACTTCAAGTCAGTTAACATTTAAC
CAGAGTTTTGAAGTAGTTGATAGCCAAGGTGGTAATCGTATGCTGGAACA
ATCAAGTTGGGGCGTGACAGCTGAAGGAGCAATCAAGCCTGATGTAACAG
CTTCTGGCTTTGAAATTTATTCTTCAACCTATAATAATCAATACCAAACA
ATGTCTGGTACAAGTATGGCTTCACCACATGTTGCAGGATTAATGACAAT
GCAATGCTTCAAAGTCATTTGGCTGAGAAATATAAAGGGATGAATTTAGA
TTCTAAAAAATTGCTAGAATTGTCTAAAAACATCCTCATGAGCTCAGCAA
CAGCATTATATAGTGAAGAGGATAAGCGTTTTATTCACCACGTCAGCAAG
GTGCAGGTGTAGTTGATGCTGAAAAAGCTATCCAAGCTCAATATTATATT
ACTGGAAACGATGGCAAAGCTAAAATTAATCTCAAACGAATGGGAGATAA
ATTTGATATCACAGTTACAATTCATAAACTTGTAGAAGGTGTCAAGAATT
GTATTATCAAGCTAATGTAGCAACAGAACAAGTAAATAAAGGTAAATTTG
CCCTTAAACCACAAGCCTTGCTAGATACTAATTGGCAGAAAGTAATTCTT
CGTGATAAAGAAACACAAGTTCGATTTACTATTGATGCTAGTCAATTTAG
TCAGAAATTAAAAGAACAGATGGCAAATGGTTATTTCTTAGAAGGTTTTG
TACGTTTTAAAGAAGCCAAGGATAGTAATCAGGAGTTAATGAGTATTCCT
TTTGTAGGATTTAATTGGTGATTTTGCGAACTTACAAGCACTTGAAACAC
CGATTTATAAGACGCTTTCTAAAGGTAGTTTCTACTATAAACCAAATGAT
ACAACTCATAAAGACCAATTGGAGTACAATGAATCAGCTCCTTTTGAAAG
CAACAACTATACTGCCTTGTTAACACAATCAGCGTCTTGGGGCTATGTTG
ATTATGTCAAAAATGGTGGGGAGTTAGAATTAGCACCGGAGAGTCCAAAA
AGAATTATTTTAGGAACTTTTGAGAATAAGGTTGAGGATAAAACAATTCA
TCTTTTGGAAAGAGATGCAGCGAATAATCCATATTTTGCCATTTCTCCAA
ATAAAGATGGAAATAGGGACGAAATCACTCCCCAGGCAACTTTCTTAAGA
AATGTTAAGGATATTTCTGCTCAAGTTCTAGATCAAAATGGAAATGTTAT
TTGGCAAAGTAAGGTTTTACCATCTTATCGTAAAAATTTCCATAATAATC
AAAGCAAAGTGATGGTCATTATCGTATGGATGCTCTTCAGTGGAGTGGTT
TAGATAAGGATGGCAAAGTTGTAGCAGATGGTTTTTATACTTATCGCTTA
CGTTACACACCAGTAGCAGAAGGAGCAAATAGTCAGGAGTCAGACTTTAA
AGTACAAGTAAGTACTAAGTCACCAAATCTTCCTTCACGAGCTCAGTTTG
ATGAAACTAATCGAACATTAAGCTTAGCCATGCCTAAGGAAAGTAGTTAT
GTTCCTACATATCGTTTACAATTAGTTTTATCTCATGTTGTAAAAGATGA
AGAATATGGGGATGAGACTTCTTACCATTATTTCCATATAGATCAAGAAG
GTAAAGTGACACTTCCTTAAAACGGTTAAGATAGGAGAGAGTGAGGTTGC
GGTAGACCCTAAGGCCTTGACACTTGTTGTGGAAGATAAAGCTGGTAATT
TCGCAACGGTAAAATTGTCTGATCTCTTGAATAAGGCAGTAGATCAGAGA
AGAAAACGCTATAGTAATTTCTAACAGTTTCAAATATTTTGATAACTTGA
AAAAAGAACCTATGTTATTTCTAAAAAAGAAAAGTAGTAAACAAGAATCT
AGAAGAAATAATATTAGTTAACCGCAAACTACAGTTACTACTCAATCATT
GTCTAAAGAAATAACTAAATCAGGAAATGAGAAGTCCTCACTTCTACAAA
CAATAATAGTAGCAGAGTAGCTAAGATCATATCACCTAAACATAACGGGG
ATTCTGTTAACCATACCTTACCTAGTACATCAGATAGAGCAACGAATGGT
CTATTTGTTGGTACTTTGGCATTGTTATCTAGTTTACTTCTTTATTTGAA
ACCCAAAAGACTAAAAATAATAGTAAA

SEQ ID NO.8

VDKHHSKKAILKLTLITTSILLMHSNQVNAEEQELKNQEQSPVIANVAQQ
PSPSVTTNTVEKTSVTAASASNTAKEMGDTSVKNDKTEDELLEELSKNLD
TSNLGADLEEEYPSKPETTNNKEESNVVTNASTAIAQKVPSAYEEVKPES
KSSLAVLDTSKITKLQAITQRGKGNVVAIIDTGFDINHDIFRLDSPKDDK
HSFKTKTEFEELKAKHNITYGKWVNDKIVFAHNYANNTETVADLAAAMKD
GYGSEAKNISHGTHVAGIFVGNSKRPAINGLLLEGAAPNAWVLLMRIPDK
IDSDKFGEAYAKAITDAVNLGAKTINMSIGKTADSLLALNDKVKLALKLA
SEKGVAVVVAAGNEGAFGMDYSKPLSTNPDYGTVNSPAISEDTLSVASYE
SLKTISEVVETTIEGKLVKLPIVTSKPFDKGKAYDVVYANYGAKKDFEGK
KIALIERGGGLDFMTKITHATNAGVVGIVIFNDQEKRGNFLIPYRELPVG
IISKVDGERIKNTSSQLTFNQSFEVVDSQGGNRMLEQSSWGVTAEGAIKP
DVTASGFEIYSSTYNNQYQTMSGTSMASPHVAGLMTMLQSHLAEKYKGMN
LDSKKLLELSKNILMSSATALYSEEDKAFYSPRQQGAGVVDAEKAIQAQY
YITGNDGKAKINLKRMGDKFDITVTIHKLVEGVKELYYQANVATEQVNKG
KFALKPQALLDTNWQKVILRDKETQVRFTIDASQFSQKLKEQMANGYFLE
GFVRFKEAKDSNQELMSIPFVGFNGDFANLQALETPIYKTLSKGSFYYKP
NDTTHKDQLEYNESAPFESNNYTALLTQSASWGYVDYVKNGGELELAPES
PKRIILGTFENDVEDKTIHLLERDAANNPYFAISPNKDGNRDEITPQATF
LRNVKDISAQVLDQNGNVIWQSKVLPSYRKNFHNNPKQSDGHYRMDALQW
SGLDKDGKVVADGFYTYRLRYTPVAEGANSQESDFKVQVSTKSPNLPSRA
QFDETNRTLSLAMPKESSYVPTYRLQLVLSHVVKDEEYGDETSYHYFHID
QEGKVTLPKTVKIGESEVAVDPKALTLVVEDKAGNFATVKLSDLLNDAVV
SEKENAIVISNSFKYFDNLKKEPMFISKKEKVVNKNLEEIILVKPQTTVT
TQSLSKEITKSGNEKVLTSTNNNSSRVAKIISPKHNGDSVNHTLPSTSDR
ATNGLFVGTLALLSSLLLYLKPKKTKNNSK

The nucleotide and amino acid sequences of GBS 173 in Ref. 3 are SEQ ID 8787 and SEQ ID 8788. These sequences are set forth below as SEQ ID NOS 9 and 10:

SEQ ID NO.9

ATGAAACGTAAATACTTTATTCTTAATACGGTGACGGTTTTAACGTTAGC
TGCTGCAATGAATACTAGCAGTATGCTAATAGTACTGAGACAAGTGCTTC
AGTAGTTCCTACTACAAATACTATCGTTCAAACTAATGACAGTAATCCTA
CCGCAAAATTTGTATCAGAATCAGGACAATCTGTAATAGGTCAAGTAAAA
CCAGATAATTCTGCGGCGCTTACAACAGTTGACACGCCTCATCATATTTC
AGCTCCAGATGCTTTAAAAACAACTCAATCAAGTCCTGTCGTTGAGAGTA
CTTCTACTAAGTTAACTGAAGAGACTTACAAACAAAAAGATGGTCAAGAT
TTAGCCAACATGGTGAGAAGTGGTCAAGTTACTAGTGAGGAACTGTTAAT
ATGGCATACGATATTATTGCTAAAGAAAACCCATCTTTAAATGCAGTCAT
TACTAGACGCCAAGAAGCTATTGAAGAGGCTAGAAAACTTAAAGATACCA
ATCAGCCGTTTTTAGGTGTTCCCTTGTTAGTCAAGGGGTTAGGGCACAGT
ATTAAAGGTGGTGAAACCAATAATGGCTTGATCTATGCAGATGAAAAATT
AGCACATTTGACAGTAGCTATGTCAAAAAATATAAAGATTTAGGATTTAT
TATTTTAGGACAAACGAACTTTCCAGAGTATGGGTGGCGTAATATAACAG
ATTCTAAATTATACGGTCTAACTGCATAATCCTTGGGATCTTGCTCATAA
TGCTGGTGGCTCTTCTGGTGGAAGTGCAGCAGCCATTGCTAGCGGAATGA
CGCCAATTGCTAGCGGTAGTGATGCTGGTGGTTCTATCCGTATTCCATCT
TCTTGGTTGAAATCACCAATGGGAACAGAAGTTAGTCAAGATGCTAAAAA
CGCTATTATGGACAACGTCACATTCTTAAGAAAACAAGGATTCAAAGTAA
CAGAGTAGACTTACCAATTGATGGTAGAGCATTAATGCGTGATTATTCAA
CCTTGGCTATTGGCATGGGAGGAGCTTTTTCAACAATTGAAAAAGACTTA
AAAAACATGGTTTTACTAAAGAAGACGTTGATCCTATTACTTGGGCAGTT
CATGTTATTTATCAAAATTCAGATAAGGCTGAACTTAAGAAATCTATTAT
GGAAGCCCAAAAACATATGGATGATTATCGTAAGGCAATGGAGAAGCTTC
ACAAGCAATTTCCTATTTTCTTATCGCCAACGACCGCAAGTTTAGCCCCT
CTAAATACAGATCCATATGTAACAGAGGAAGATAAAAGAGCGATTTATAA
TATGGAAACTTGAGCCAAGAAGAAAGAATTGCTCTCTTTAATCGCCAGTG
GGAGCCTATGTTGCGTAGAACACCTTTTACACAAATTGCTAATATGACAG
GACTCCCAGCTATCAGTATCCCGACTTACTTATCTGAGTCTGGTTTACCC
ATAGGGACGATGTTAATGGCAGGTGCAAACTATGATATGGTATTAATTAA
ATTTGCAACTTTCTTTGAAAAACATCATGGTTTTAATGTTAAATGGCAAA
GAATAATAGATAAAGAAGTGAAACCATCTACTGGCCTAATACAGCCTACT
AACTCCCTCTTTAAAGCTCATTCATCATTAGTAAATTTAGAAGAAAATTC
ACAAGTTACTCAAGTATCTATCTCTAAAAAATGGATGAAATCGTCTGTTA
AAAATAAACCATCCGTAATGGCATATCAAAAGCACTTCCTAAAACAGGTG
ATACAGAATCAAGCCTATCTCCAGTTTTAGTAGTAACCCTTTTATTAGCT
TGTTTTAGCTTTGTAACAAAAAGAATCAGAAAAGT

SEQ ID NO.10

MKRKYFILNTVTVLTLAAAMNTSSIYANSTETSASVVPTTNTIVQTNDSN
PTAKFVSESGQSVIGQVKPDNSAALTTVDTPHHISAPDALKTTQSSPVVI
STSTKLTEETYKQKDGQDLANMVRSGQVTSEELVNMAYDIIAKENPSLNA
VITTRRQEAIEEARKLKDTNQPFLGVPLLVKGLGHSIKGGETNNGLIYAD
GKISTFDSSYVKKYKDLGFIILGQTNFPEYGWRNITDSKLYGLTHNPWDL
AHNAGGSSGGSAAAIASGMTPIASGSDAGGSIRIPSSWTGLVGLKPTRGL
VSNEKPDSYSTAVHFPLTKSSRDAETLLTYLKKSDQTLVSVSNDLKSLPI
AYTLKSPMGTEVSQDAKNAIMDNVTFLRKQGFKVTEIDLPIDGRALMRDY
STLAIGMGGAFSTIEKDLKKHGFTKEDVDPITWAVHVIYQNSDKAELKKS
IMEAQKHMDDYRKAMEKLHKQFPIFLSPTTASLAPLNTDPYVTEEDKRAI
YNMENLSQEERIALFNRQQWEPMLRRTPFTQIANMTGLPAISIPTYLESG
LPIGTMLMAGANYDMVLIKATFFEKHHGFNVKWQRIIDKEVKPSTGLIQP
TNSLFKAHSSLVNLEENSQVTQVSISKKWMKSSVKNKPSVMAYQKALPKT
GDTESSLSPVLVVTLLLACFSFVTKKNQKS

The nucleotide and amino acid sequences of GBS 276 in Ref. 3 are SEQ ID 8941 and SEQ ID 8942. These sequences are set forth below as SEQ ID NOS 11 and 12:

SEQ ID NO.11

TTGCGTAAAAAACAAAAACTACCATTTGATAAACTTGCCATTGCGCTTAT
ATCTACGAGCATCTTGCTCAATGCACAATCAGACATTAAAGCAAATACTG
TGACAGAAGACACTCCTGCTACCGAACAAGCCGTAGAACCCCCACAACCA
ATAGCAGTTTCTGAGGAATCACGATCATCAAAGGAAACTAAAACCTCACA
AACTCCTAGTGATGTAGGAGAAACAGTAGCAGATGACGCTAATGATCTAG
CCCCTCAAGCTCCTGCTAAAACTGCTGATACACCAGCAACCTCAAAAGCG
ACTATTAGGGATTTGAACGACCCTTCTCATGTCAAAACCCTGCAGGAAAA
AGCAGGCAAGGGAGCTGGGACCGTTGTTGCAGTGATTGATGCTGGTTTTG
ATAAAAATCATGAAGCGTGGCGCTTAACAGACAAAACTAAAGCACGTTAC
CAATCAAAAGAAAATCTTGAAAAAGCTAAAAAAGAGCACGGTATTACCTA
TGGCGAGTGGGTCAATGATAAGGTTGCTTATTACCACGACTATAGTAAAG
ATGGTAAAAACGCTGTTGATCAAGAACACGGCACACACGTGTCAGGGATC
TTGTCAGGAAATGCTCCATCTGAAATGAAAGAACCTTACCGCCTAGAAGG
TGCGATGCCTGAGGCTCAATTGCTTTTGATGCGTGTCGAAATTGTAAATG
GACTAGCAGACTATGCTCGTAACTACGCTCAAGCTATCAGAGATGCTGTC
AACTTGGGAGCTAAGGTGATTAATATGAGCTTTGGTAATGCTGCACTAGC
TTACGCCAACCTTCCAGACGAAACCAAAAAAGCCTTTGACTATGCCAAAT
CAAAAGGTGTTAGCATTGTGACCTCAGCTGGTAATGATAGTAGCTTTGGG
GGCAAGCCCCGTCTACCTCTAGCAGATCATCCTGATTATGGGGTGGTTGG
GACACCTGCAGCGGCAGATTCAACATTGACAGTTGCTTCTTACAGCCCAG
ATAAACAGCTCACTGAAACTGCTACGGTCAAAACAGACGATCATCAAGAT
AAAGAAATGCCTGTTATTTCAACAAACCGTTTTGAGCCAAACAAGGCTTA
CGACTATGCTTATGCTAATCGTGGTACGAAAGAGGATGATTTTAAGGATG
TCGAAGGTAAGATTGCCCTTATTGAACGTGGCGATATTGATTTCAAAGAT
AAGATTGCAAACGCTAAAAAAGCTGGTGCTGTAGGGGTCTTGATCTATGA
CAATCAAGACAAGGGCTTCCCGATTGAATTGCCAAATGTTGACCAGATGC
CTGCGGCCTTTATCAGTCGAAGAGACGGTCTCTTATTAAAAGACAATCCC
CCAAAAACCATTACCTTCAATGCGACACCTAAGGTATTGCCAACAGCAAG
TGGCACCAAACTAAGCCGCTTCTCAAGCTGGGGTCTGACAGCTGACGGCA
ATATTAAACCGGATATTGCAGCACCCGGCCAAGATATTTTGTCATCAGTG
GCTAACAACAAGTATGCCAAACTTTCTGGAACTAGTATGTCTGCACCATT
GGTAGCGGGTATCATGGGACTGTTGCAAAAGCAATATGAGACACAGTATC
CTGATATGACACCATCAGAGCGTCTTGATTTAGCTAAGAAAGTATTGATG
AGCTCAGCAACTGCCCTATATGATGAAGATGAAAAAGCTTATTTTTCTCC
TCGCCAACAGGGAGCAGGAGCAGTCGATGCTAAAAAAGCTTCAGCAGCAA
CGATGTATGTAACAGATAAGGACAATACCTCAAGCAAGGTTCACCTGAAC
AATGTTTCTGATAAATTTGAAGTAACAGTAAAGTTCACAACAAATCTGAT
AAACCTCAAGAGTTGTATTACCAAGTAACTGTTCAAACAGATAAAGTAGA
TGGAAAACACTTTGCCTTGGCTCCTAAAGCATTGTATGAGACATCATGGC
AAAAATCACAATTCCAGCCAATAGCAGCAAACAAGTCACCGTTCCAATCG
ATGCTAGTCGATTTAGCAAGGACTTGCTTGCCCAAATGAAAAATGGCTAT
TTCTTAGAAGGTTTTGTTCGTTTCAAACAAGATCCTACAAAAGAAGAGCT
TATGAGCATTCCATATATTGGTTTCCGAGGTGATTTTGGCAATCTGTCAG
CCTTAGAAAAACCAATCTATGATAGCAAAGACGGTAGCAGCTACTATCAT
GAAGCAAATAGTGATGCCAAAGACCAATTAGATGGTGATGGATTACAGTT
TTACGCTCTGAAAAATAACTTTACAGCACTTACCACAGAGTCTAACCCAT
GGACGATTATTAAAGCTGTCAAAGAAGGGGTTGAAAACATAGAGGATATC
GAAATCTTCAGAGATCACAGAAACCATTTTTGCAGGTACTTTTGCAAAAC
AAGACGATGATAGCCACTACTATATCCACCGTCACGCTAATGGCAAACCA
TATGCTGCGATCTCTCCAAATGGGGACGGTAACAGAGATTATGTCCAATT
CCAAGGTACTTTCTTGCGTAATGCTAAAAACCTTGTGGCTGAAGTCTTGG
ACAAAGAAGGAAATGTTGTTTGGACAAGTGAGGTAACCGAGCAAGTTGTT
AAAAACTACAACAATGACTTGGCAAGCACACTTGGTTCAACCCGTTTTGA
AAAACGCGTTGGGACGGTAAAGATAAAGACGGCAAAGTTGTTGCTAACGG
AACCTACACCTATCGTGTTCGCTACACGCCGATTAGCTCAGGTGCAAAAG
AACAACACACTGATTTTGATGTGATTGTAGACAATACGACACCTGAAGTC
GCAACATCGGCAACATTCTCAACAGAAGATAGTCGTTTGACACTTGCATC
TAAACCAAAAACCAGCCAACCGGTTTACCGTGAGCGTATTGCTTACACTT
ATATGGATGAGGATCTGCCAACAACAGAGTATTTCTCCAAATGAAGATGG
TACCTTTACTCTTCCTGAAGAGGCTGAAACAATGGAAGGCGCTACTGTTC
CATTGAAAATGTCAGACTTTACTTATGTTGTTGAAGATATGGCTGGTAAC
ATCACTTATACACCAGTGACTAAGCTATTGGAGGGCCACTCTAATAAGCC
AGAACAAGACGGTTCAGATCAAGCACCAGACAAGAAACCAGAAGCTAAAC
CAGAACAAGACGGTTCAGGTCAAACACCAGTAAAAAAGAAACTAAACCAG
AAAAAGATAGTTCAGGTCAACAACCAGGTAAAACTCCTCAAAAGGTCAAT
CTTCTCGTACTCTAGAGAAACGATCTTCTAAGCGTGTTTAGCTACAAAAG
CATCAACAAGAGATCAGTTACCAACGACTAATGACAGGATACAAATCGTT
TACATCTCCTTAAGTTAGTTATGACCACTTTCTTCTTGGGA

SEQ ID NO.12

MRKKQKLPFDKLAIALISTSILLNAQSDIKANTVTEDTPATEQAVEPPQP
IAVSEESRSSKETKTSQTPSDVGETVADDANDLAPQAPAKTADTPATSKA
TIRDLNDPSHVKTLQEKAGKGAGTVVAVIDAGFDKNHEAWRLTDKTKARY
QSKENLEKAKKEHGITYGEWVNDKVAYYHDYSKDGKNAVDQEHGTHVSGI
LSGNAPSEMKEPYRLEGAMPEAQLLLMRVEIVNGLADYARNYAQAIRDAV
NLGAKVINMSFGAALAYANLPDETKKAFDYAKSKGVSIVTSAGNDSSFGG
KPRLPLADHPDYGVVGTPAAADSTLTVASYSPDKQLTETATVKTDDHQDK
EMPVISTNRFEPNKAYDYAYANRGTKEDDFKDVEGKIALIERGDIDFKDK
IANAKKAGAVGVLIYDNQDKGFPIELPNVDQMPAAFISRRDGLLLKDNPP
KTITFNATPKVLPTASGTKLSRFSSWGLTADGNIKPDIAAGQDILSSVAN
NKYAKLSGTSMSAPLVAGIMGLLQKQYETQYPDMTPSERLDLAKKVLMSS
ATALYDEDEKAYFSPRQQGAGAVDAKKASAATMYVTDKDNTSSKVHLNNV
SDKFEVTVTVHNKSDKPQELYYQVTVQTDKVDGKHFALAPKALYETSWQK
ITIPANSSKQVTVPIDASRFSKDLLAQMKNGYFLEGFVRFKQDPTKEELM
SIPYIGFRGDFGNLSALEKPIYDSKDGSSYYHEANSDAKDQLDGDGLQFY
ALKNNFTALTTESNPWTIIKAVKEFVENIEDIESSEITETIFAGTFAKQD
DDSHYYIHRHANGKPYAAISPNGDGNRDYVQFQGTFLRNAKNLVAEVLDK
EGNVVWTSEVTEQVVKNYNNDLASTLGSTRFEKTRWDGKDKDGKVVANGT
YTYRVRYTPISSGAKEQHTDFDVIVDNTTPEVATSATFSTEDSRLTLASK
PKTSQPVYRERIAYTYMDEDLPTTEYISPNEDGTFTLPEEAETMEGATVP
LKMSDFTYVVEDMAGNITYTPPVTKLLEGHSNKPEQDGSDQAPDKKPEAK
PEQDGSGQTPDKKKETKPEKDSSGQTPGKTPQKGQSSRTLEKRSSKRALA
TKASTRDQLPTTNDKDTNRLHLLKLVMTTFFLG

The nucleotide and amino acid sequences of GBS 305 in Ref. 3 are SEQ ID 207 and SEQ ID 208. These sequences are set forth below as SEQ ID NOS 13 and 14:

SEQ ID NO.13

ATGGGACGAGTAATGAAAACAATAACAACATTTGAAAATAAAAAAGTTTT
AGTCCTTGGTTTAGCACGATCTGGAGAAGCTGCTGCACGTTTGTTAGCTA
AGTTAGGAGCAATAGTGACAGTTAATGATGGCAAACCATTTGATGAAAAT
CCAACAGCACAGTCTTTGTTGGAAGAGGGTATTAAAGTGGTTTGTGGTAG
TCATCCTTTAGAATGTTAGATGAGGATTTTTGTTACATGATTAAAAATCC
AGGAATACCTTATAACAATCCTATGGTCAAAAAAGCATTAGAAAAACAAA
TCCCTGTTTTGACTGAAGTGGAATTAGCATACTTAGTTTCAGAATCTCAG
CTAATAGGTATTACAGGCTCTAACGGGAAAACGACAACGACAACGATGAT
TGCAGAAGTCTTAAATGCTGGAGGTCAGAGAGGTTTGTTAGCTGGGAATA
TCGGCTTTCCTGCTAGTGAAGTTGTTCAGGCTGCGAATGATAAAGATACT
CTAGTTATGGAATTATCAAGTTTTCAGCTAATGGGAGTTAAGGAATTTCG
TCCTCATATTGCAGTAATTACTAATTTAATGCCAACTCATTTAGATTATC
ATGGGTCTTTTGAAGATTATGTTGCTGCAAAATGGAATATCCAAAATCAA
ATGTCTTCATCTGATTTTTTGGTACTTAATTTTAATCAAGGTATTTCTAA
AGAGTTAGCTAAAACTACTAAAGCAACAATCGTTCCTTTCTCTACTACGA
AAAAAGTTGATGGTGCTTACGTACAAGACAAGCAACTTTTCTATAAAGGG
GAGAATATTATGTCAGTAGATGACATTGGTGTCCCAGGAAGCCATAACGT
AGAGAATGCTCTAGCAACTATTGCGGTTGCTAAACTGGCTGGTATCAGTA
ATCAAGTTATTAGAGAAACTTTAAGCAATTTTGGAGGTGTTAAACACCGC
TTGCAATCACTCGGTAAGGTTCATGGTATTAGTTTCTATAACGACAGCAA
GTCAACTAATATATTGGCTCAAAAAGCATTATCTGGCTTTGATAATACTA
AAGTTATCCTAATTGCAGGAGGTCTTGATCGCGGTAATGAGTTTGATGAA
TTGATACCAGATATCACTGGACTTAAACATATGGTTGTTTTAGGGGAATC
GGCATCTCGAGTAAAACGTGCTGCACAAAAAGCAGGATAACTTATAGCGA
TGCTTTAGATGTTAGAGATGCGGTACATAAAGCTTATGAGGTGGCACAAC
AGGGCGATGTTATCTTGCTAAGTCCTGCAAATGCATCATGGGACATGTAT
AAGAATTTCGAAGTCCGTGGTGATGAATTCATTGATACTTTCGAAAGTCT
TAGAGGAGAG

SEQ ID NO.14

MGRVMKTITTFENKKVLVLGLARSGEAAARLLAKLGAIVTVNDGKPFDEN
PTAQSLLEEGIKVVCGSHPLELLDEDFCYMIKNPGIPYNNPMVKKALEKQ
IPVLTEVELAYLVSESQLIGITGSNGKTTTTTMIAEVLNAGGQRGLLAGN
IGFPASEVVQAANDKDTLVMELSSFQLMGVKEFRPHIAVITNLMPTHLDY
HGSFEDYVAAKWNIQNQMSSSDFLVLNFNQGISKELAKTTKATIVPFSTT
EKVDGAYVQDKQLFYKGENIMSVDDIGVPGSHNVENALATIAVAKLAGIS
NQVIRETLSNFGGVKRLQSLGKVHGISFYNDSKSTNILATQKALSGFDNT
KVILIAGGLDRGNEFDELIPDITGLKHMVVLGESASRVKRAAQKAGVTYS
DALDVRADAVHKAYEVAQQGDVILLSPANASWDMYKNFEVRGDEFIDTFE
SLRGE

The nucleotide and amino acid sequences of GBS 313 are in Ref. 3 are SEQ ID 4089 and SEQ ID 4090. These sequences are set forth as SEQ ID NOS 15 and 16 below:

SEQ ID NO.15

ATGAAACGTATTGCTGTTTTAACTAGTGGTGGTGACGCCCCTGGTATGAA
CGCTGCTATCCGTGCAGTTGTTCGTAAAGCAATTTCTGAAGGTATGGAAG
TTTACGGCATCAACCAAGGTTACTATGGTATGGTGACAGGGGATATTTTC
CCTTTGGATGCTAATTCTGTTGGGGATACTATCAACCGTGGAGGAACGTT
TTTACGTTCAGCACGTTATCCTGAATTTGCTGAACTTGAAGGTCAGCTTA
AAGGGATTGAACAGCTTAAAAAACACGGTATTGAAGGTGTAGTAGTTATC
GGTGGTGATGGTTCTTATCATGGTGCTATGCGTCTAACTGAGCACGGTTT
CCCAGCTGTTGGTTTGCCGGGTACAATTGATAACGATATCGTTGGCACTG
ACTATACTATTGGTTTTGACACAGCAGTTGCGACAGCAGTTGAGAATCTT
GACCGTCTTCGTGATACATCAGCAAGTCATAACCGTACTTTTGTTGTTGA
GGTTATGGGAAGAAATGCAGGAGATATCGCTCTTTGGTCAGGTATCGCTG
CAGGTGCAGATCAAATTATTGTTCCTGAAGAAGAGTTCAATATTGATGAA
GTTGTCTCAAATGTTAGAGCTGGCTATGCAGCTGGTAAACATCACCAAAT
CATCGTCCTTGCAGAAGGTGTTATGAGTGGTGATGAGTTTGCAAAAACAA
TGAAAGCAGCAGGAGACGATAGCGATCTTCGTGTGACGAATTTAGGACAT
CTGCTCCGTGGTGGTAGTCCGACGGCTCGTGATCGTGTCTTAGCATCTCG
TATGGGAGCGTACGCTGTTCAATTGTTGAAAGAAGGTCGTGGTGGTTTAG
CCGTTGGTGTCCACAACGAAGAAATGGTTGAAAGTCCAATTTTAGGTTTA
GCAGAAGAAGGTGCTTTGTTCAGCTTGACTGATGAAGGAAAAATCGTTGT
TAATAATCCGCATAAAGCGGACCTTCGCTTGGCAGCACTTAATCGTGACC
TTGCCAACCAAAGTAGTAAA

SEQ ID NO.16

MKRIAVLTSGGDAPGMNAAIRAVVRKAISEGMEVYGINQGYYGMVTGDIF
PLDANSVGDTINRGGTFLRSARYPEFAELEGQLKGIEQLKKHGIEGVVVI
GGDGSYHGAMRLTEHGFPAVGLPGTIDNDIVGTDYTIGFDTAVATAVENL
DRLRDTSASHNRTFVVEVMGRNAGDIALWSGIAAGADQIIVPEEEFNIDE
VVSNVRAGYAAGKHHQIIVLAEGVMSGDEFAKTMKAAGDDSDLRVTNLGH
LLRGGSPTARDRVLASRMGAYAVQLLKEGRGGLAVGVHNEEMVESPILGL
AEEGALFSLTDEGKIVVNNPHKADLRLAALNRDLANQSSK

The nucleotide and amino acid sequences of GBS 322 in Ref. 3 are SEQ ID 8539 and SEQ ID 8540. These sequences are set forth below as SEQ ID NOS 17 and 18:

SEQ ID NO.17

ATGAATAAAAGGTACTATTGACATCGACAATGGCAGCTTCGCTATTATCA
GTCGCAAGTGTTCAAGCACAAGAAACAGATACGACGTGGACAGCACGTAC
TGTTTCAGAGGTAAAGGCTGATTTGGTAAAGCAAGACAATAAATCATCAT
ATACTGTAAATATGGTGATACACTAAGCGTTATTTCAGAAGCAATGTCAA
TTGATATGAATGTCTTAGCAAAAATAAATAACATTGCAGATATCAATCTT
ATTTATCCTGAGACAACACTGACAGTAACTTACGATCAGAAGAGTCATAC
TGCCACTTCAATGAAAATAGAAACACCAGCAACAAATGCTGCTGGTCAAA
CAACAGCTACTGTGGATTTGAAAACCAATCAAGTTTCTGTTGCAGACCAA
AAGTTTCTCTCAATACAATTTCGGAAGGTATGACACCAGAAGCAGCAACA
ACGATTGTTTCGCCAATGAAGACATATTCTTCTGCGCCAGCTTTGAAATC
AAAAGAAGTATTAGCACAAGAGCAAGCTGTTAGTCAAGCAGCAGCTAATG
AACAGGTATCACCAGCTCCTGTGAAGTCGATTACTTCAGAAGTTCCAGCA
GCTAAAGAGGAAGTTAAACCAACTCAGACGTCAGTCAGTCAGTCAACAAC
AGTATCACCAGCTTCTGTTGCCGCTGAAACACCAGCTCCAGTAGCTAAAG
TAGCACCGGTAAGAACTGTAGCAGCCCCTAGAGTGGCAAGTGTTAAAGTA
GTCACTCCTAAAGTAGAAACTGGTGCATCACCAGAGCATGTATCAGCTCC
AGCAGTTCCTGTGACTACGACTTCACCAGCTACAGACAGTAAGTTACAAG
CGACTGAAGTTAAGAGCGTTCCGGTAGCACAAAAAGCTCCAACAGCAACA
CCGGTAGCACAACCAGCTTCAACAACAAATGCAGTAGCTGCACATCCTGA
AAATGCAGGGCTCCAACCTCATGTTCAGCTTATAAAGAAAAAGTAGCGTC
AACTTATGGAGTTAATGAATTCAGTACATACCGTGCGGAGATCCAGGTGA
TCATGGTAAAGGTTTAGCAGTTGACTTTATTGTAGGTACTAATCAAGCAC
TTGGTAATAAAGTTGCACAGTACTCTACACAAAATATGGCAGCAAATAAC
ATTTCATATGTTATCTGGCAACAAAAGTTTTACTCAAATACAAACAGTAT
TTATGGACCTGCTAATACTTGGAATGCAATGCCAGATCGTGGTGGCGTTA
CTGCCAACCACTATGACCACGTTCACGTATCATTTAACAAATAATATAAA
AAAGGAAGCTATTTGGCTTCTTTTTTATATGCCTTGAATAGACTTTCAAG
GTTCTTATATAATTTTTATTA

SEQ ID NO.18

MNKKVLLTSTMAASLLSVASVQAQETDTTWTARTVSEVKADLVKQDNKSS
YTVKYGDTLSVISEAMSIDMNVLAKINNIADINLIYPETTLTVTYDQKSH
TATSMKIETPATNAAGQTTATVDLKTNQVSVADQKVSLNTISEGMTPEAA
TTIVSPMKTYSSAPALKSKEVLAQEQAVSQAAANEQVSPAPVKSITSEVP
AAKEEVKPTQTSVSQSTTVSPASVAAETPAPVAKVAPVRTVAAPRVASVK
VVTPKETGASPEHVSAPAVPVTTTSPATDSKLQATEVKSVPVAQKAPTAT
PVAQPASTTNAVAAHPENAGLQPHVAAYKEKVASTYGVNEFSTYRAGDPG
DPGDHGKGLAVDFIVGTNQALGNKAQYSTQNMAANNISYVIWQQKFYSNT
NSIYGPANTWNAMPDRGGVTANHYDHVHVSFNK

The nucleotide and amino acid sequences of GBS 328 in Ref. 3 are SEQ ID 6015 and SEQ ID 6016. These sequences are set forth below as SEQ ID NOS 19 and 20:

(SEQ ID NO.19)

ATGAAAAGAAAATTATTTTGAAAAGTAGTGTTCTTGGTTTAGTCGCTGGG
ACTTCTATTATGTTCTCAAGCGTGTTCGCGGACCAAGTCGGTGTCCAAGT
TATAGGCGTCAATGACTTTCATGGTGCACTTGACAATACTGGAACAGCAA
ATATGCCTGATGGAAAAGTTGCTAATGCTGGTACTGCTGCTCAATTAGAT
GCTTATATGGATGACGCTCAAAAAGATTTCAAACAAACTAACCCTAATGG
TGAAAGCATTAGGGTTCAAGCAGGCGATATGGTTGGAGCAAGTCCAGCCA
ACTCTGGGCTTCTTCAAGATGAACCAACTGTCAAAAATTTTAATGCAATG
AATGTTGAGTATGGCACATTGGGTAACCATGAATTTGATGAAGGGTTGGC
AGAATATAATCGTATCGTTACTGGTAAAGCCCCTGCTCCAGATTCTAATA
TTAATAATATTACGAAATCATACCCACATGAAGCTGCAAAACAAGAAATT
GTAGTGGCAAATGTTATTGATAAAGTTAACAAACAAATTCCTTACAATTG
GAAGCCTTACGCTATTAAAAATATTCCTGTAAATAACAAAAGTGTGAACG
TTGGCTTTATCGGGATTGTCACCAAGACATCCCAAACCTTGTCTTACGTA
AAAATTATGAACAATATGAATTTTTAGATGAAGCTGAAACAATCGTTAAA
TACGCCAAAGAATTACAAGCTAAAAATGTCAAAGCTATTGTAGTTCTCGC
ACATGTACCTGCAACAAGTAAAAATGATATTGCTGAAGGTGAAGCAGCAG
AAATGATGAAAAAAGTCAATCAACTCTTCCCTGAAAATAGCGTAGATATT
GTCTTTGCTGGACACAATCATCAATATACAAATGGTCTTGTTGGTAAAAC
TCGTATTGTACAAGCGCTCTCTCAAGGAAAAGCCTATGCTGATGTACGTG
GTGTCTTAGATACTGATACACAAGATTTCATTGAGACCCCTTCAGCTAAA
GTAATTGCAGTTGCTCCTGGTAAAAAAACAGGTAGTGCCGATATTCAAGC
CATTGTTGACCAAGCTAATACTATCGTTAAACAAGTAACAGAAGCTAAAA
TTGGTACTGCCGAGGTAAGTGTCATGATTACGCGTTCTGTTGATCAAGAT
AATGTTAGTCCGGTAGGCAGCCTCATCACAGAGGCTCAACTAGCAATTGC
TCGAAAAAGCTGGCCAGATATCGATTTTGCCATGACAAATAATGGTGGCA
TTCGTGCTGACTTACTCATCAAACCAGATGGAACAATCACCTGGGGAGCT
GCACAAGCAGTTCAACCTTTTGGTAATATCTTACAAGTCGTCGAAATTAC
TGGTAGAGATCTTTATAAAGCACTCAACGAACAATACGACCAAAAACAAA
ATTTCTTCCTTCAAATAGCTGGTCTGCGATACACTTACACAGATAATAAA
GAGGGCGGGGAAGAAACACCATTTAAAGTTGTAAAAGCTTATAAATCAAA
TGGTGAGGAAATCAATCCTGATGCAAATACAAATTAGTTATCAATGACTT
TTTATTCGGTGGTGGTGATGGCTTTGCAAGCTTCAGAAATGCCAAACTTC
TAGGAGCCATTAACCCCGATACAGAGGTATTTATGGCCTATATCACATGA
TTTAGAAAAAGCTGGTAAAAAAGTGAGCGTTCCAAATAATAAACCTAAAA
TCTATGTCACTATGAAGATGGTTAATGAAACTATTACACAAAATGATGGT
ACACATAGCATTATTAAGAAACTTTATTTAGATCGACAAGGAAATATTGT
AGCACAAGAGATTGTATCAGACACTTTAAACCAAAACAAAATCAAAATCT
ACAAAAATCAACCCTGTAACTACAATTCACAAAAAACAATTACACCAATT
TACAGCTATTAACCCTATGAGAAATTATGGCAAACCATCAAACTCCACTA
CTGTAAAATCAAAACAATTACCAAAAACAAACTCTGAATATGGACAATCA
TTCCTTATGTCTGTCTTTGGTGTTGGACTTATAGGAATTGCTTTAAATAC
AAAGAAAAAACATATGAAA

SEQ ID NO.20

MKKKIILKSSVLGLVAGTSIMFSSVFADQVGVQVIGVNDFHGALDNTGTA
MMPDGKVANAGTAAQLDAYMDDAQKDFKQTNPNGESIRVQAGDMVGASPA
NSGLLQDEPTVKNAMNVEYGTLGNHEFDEGLAEYNRIVTGKAPAPDSNIN
NITKSYPHEAAKQEIVVANVIDKVNKQIPYNWKPYAIKNIPVNNKSVNVG
FIGIVTKDIPNLVLRKNYEQYEFLDEAETIVKYAKELQAKNVKAIVVLAH
VPATSKNDLAEGEAAEMMKKVNQLFPENSVDIVFAGHNHQYTNGLVGKTR
IVQALSQGKAYADVRGVLDTDTQDFIETPSAKVIAVAPGKKTGSADIQAI
VDQANTIVKQVTEAKIGTAEVSVMITRSVDQDNVSPVGSLITEAQLAIAR
KSWPDIDFAMTNNGGIRADLLIKPDGTITWGAAQAVQPFGNILQVVEITG
RDLYKALNEQYDQKQNFFLQLAGLRYTYTDNKEGGEETPFKVVKAYKSNG
EEINPKAKYKLVINDFLFGGGDFASFRNAKLLGAINPDTEVFMAYITDLE
KAGKKVSVPNNKPKIYVTMKMKMVNETITQNDGTHSIIKKLYLKRQGNIV
AQEIVSDTLNQTKSKSTKINPVTTIHKKQLHQFTAINPMRNYGKPSNSTT
VKSKQLPKTNSEYGQSFLMSVFGVGLIGIALNTKKKHMK

The nucleotide and amino acid sequences of GBS 330 in Ref. 3 are SEQ ID 8791 and SEQ ID 8792. These sequences are set forth below as SEQ ID NOS 21 and 22:

SEQ ID NO.21

ATGAATAAACGCGTAAAAATCGTTGCAACACTTGGTCCTGCGGTTGAATT
CCGTGGTGGTAAGAAGTTTGGTGAGTCTGGATACTGGGGTGAAAGCCTTG
ACTAGAAGCTTCAGCAGAAAAAATTGCTCAATTGATTAAAGAAGGTGCTA
ACGTTTTCCGTTTCAACTTCTCACATGGAGATCATGCTGAGCAAGGAGCT
CGTATGGCTACTGTTCGTAAAGCAGAAGAGATTGCAGGACAAAAAGTTGG
CTTCCTCCTTGATACTAAAGGACCTGAAATTCGTACAGAACTTTTTGAAG
ATGGTGCAGATTTCCATTCATATACAACAGGTACAAAATTACGTGTTGCT
ACTAAGCAAGGTATCAAATCAACTCCAGAAGTGATTGCATTGAATGTTGC
TGGTGGACTTGACATCTTTGATGACGTTGAAGTTGGTAAGCAAATCCTTG
TTGATGATGGTAAACTAGGTCTTACTGTGTTTGCAAAAGATAAAGACACT
CGTGAATTTGAAGTAGTTGTTGAGAATGATGGCCTTATTGGTAAACAAAA
AGGTGTAAACATCCCTTATACTAAAATTCCTTTCCCAGCACTTGCAGAAC
GCGATAATGCTGATATCCGTTTTGGACTTGAGCAAGGACTTAACTTTATT
GCTATCTCATTTGTACGTACTGCTAAAGATGTTAATGAAGTTCGTGCTAT
TTGTGAAGAAACTGGSMATGGACACGTTAAGTTGTTTGCTAAAATTGAAA
ATCAACAAGGTATCGATAATATTGATGAGATTATCGAAGCAGATGGTATT
ATGATTGCTCGTGGATATGGGTATCGAAGTTCCATTTGAAATGGTTCCAG
TTTACCAAAAAATGATCATTACTAAAGTAATGCAGCTGGTAAAGCAGTTA
TTACAGCAACAAATATGCTTGAAACAATGACTGATAAACCACGTGCGACT
CGTTCAGAAGTATCTGATGTCTTCAATGCTGTTATTGATGGTACTGATGC
TACAATGCTTTCAGGTGAGTCAGCTAATGGTAAATACCCAGTTGAGTCAG
TTCGTACAATGGCTACTATTGATAAAAATGCTCAAACATTACTCAATGAG
TATGGTCGCTTAGACTCATCTGCATTCCCACGTAATAACAAAACTGATGT
TATTGCATCTGCGGTAAAGATGCAACACACTCAATGGATATCAAACTTGT
TGTAACAATTACTGAAACAGGTAATACAGCTCGTGCCATTTCTAAATTCC
GTCCAGATGCAGACATTTTGGCTGTTACATTTGATGAAAAAGTACAACGT
TCATTGATGATTAACTGGGGTGTTATCCCTGTCCTTGCAGACAAACCAGC
ATCTACAGATGATATGTGTTTAGGTTGCAGAACGTGTAGCACTTGAAGCA
GGATTTGTTGAATCAGGCGATAATATCGTTATCGTTGCAGGTGTTCCTGT
AGGTACAGGTGGAACTAACACAATGCGTGTTCGTACTGTTAAA

SEQ ID NO.22

MNKRVKIVATLGPAVEFRGGKKFGESGYWGESLDVEASAEKIAQLIKEGA
NVFRFNFSHGDHAEQGARMATVRKAEEIAGQKVGFLLDTKGPEIRTELFE
DGADFHSYTTGTKLRVATKQGIKSTPEVIALNVAGGLDIFDDVEVGKQIL
VDDGKLGLTVFAKDKDTREFEVVVENDGLIGKQKGVNIPYTKIPFPALAE
RDNADIRFGLEQGLNFIAISFVRTAKDVNEVRAICEETGXGHVKLFAKIE
NQQGIDNIDEIIEAADGIMIARGDMGIEVPFEMVPVYQKMIITKVNAAGK
AVITATNMLETMTDKPRATRSEVSDVFNAVIDGTDATMLSGESANGKYPV
ESVRTMATIDKNQTLLNEYGRLDSSAFPRNNKTDVIASAVKDATHSMDIK
LVVTITETGNTARAISKFRPDADILAVFDEKVQRSLMINWGVIPVLADKP
ASTDDMFEVAERVALEAGEVESGDNIVIVAGVPVGTGGTNTMRVRTVK

The nucleotide and amino acid sequences of GBS 338 in Ref. 3 are SEQ ID 8637 and SEQ ID 8638. These sequences are set forth below as SEQ ID NOS 23 and 24:

SEQ ID NO.23

TTGTCTGCTATAATAGACAAAAAGGTGGTGATATTTATGTATTTAGCATT
AATCGGTGATATCATTAATTCAAAACAGATACTTGAACGTGAAACTTTCC
AACAGTCTTTTCAGCAACTAATGACCGAACTATCTGATGTATATGGTGAA
GAGCTGATTTCTCCATTCACTATTACAGCTGGTGATGAATTTCAAGCTTT
ATTGAAACCATCAAAAAAGGTATTTCAAATTATTGACCATATTCAACTAG
CTCTAAAACCTGTTAATGTAAGGTTCGGCCTCGGTACAGGAAACATTATA
ACATCCATCAATTCAAATGAAAGTATCGGTGCTGATGGTCCTGCCTACTG
GCATGCTCGCTCAGCTATTAATCATATACATGATAAAAATGATTATGGAA
CAGTTCAAGTAGCTATTTGCCTTGATGATGAAGACCAAAACCTTGAATTA
ACACTAAATAGTCTCATTTCAGCTGGTGATTTTATCAAGTCAAAATGGAC
TACAAACCATTTTCAAATGCTTGAGCACTTAATACTTCAAGATAATTATC
AAGAACAATTTCAACATCAAAAGTTAGCCCAACTGGAAAATATTGAACCT
AGTGCGCTGACTAAACGCCTTAAAGCAAGCGGTCTGAAGATTTACTTAAG
AACGAGAACACAGGCAGCCGATCTATTAGTTAAAAGTTGCACTCAAACTA
AAGGGGGAAGCTATGATTTC

SEQ ID NO.24

MSAIIDKKVVIFMYLALIGDIINSKQILERETFQQSFQQLMTELSDVYGE
ELISPFTITAGDEFQALLKPSKKVFQIIDHIQLALKPVNVRFGLGTGNII
TSINSNESIGADGPAYWHARSAINHIHDKNDYGTVQVAICLDDEDQNLEL
TLNSLISAGDFIKSKWTTNHFQMLEHLILQDNYQEQFQHQLAQLENIEPS
ALTKRLKASGLKIYLRTRTQAADLLVKSCTQTKGGSYDF

The nucleotide and amino acid sequences of GBS 358 in Ref. 3 are SEQ ID 3183 and SEQ ID 3184. These sequences are set forth below as SEQ ID NOS 25 and 26:

SEQ ID NO.25

ATGTTTTATACAATTGAAGAGCTGGTAGAGCAAGCTAATAGCCAACATAA
GGGTAACATAGCAGAGCTCATGATCCAAACGGAAATTGAAATGACTGGTA
GAAGTCGTGAAGAAATTCGTTATATTATGTCCCGAAATCTTGAAGTCATG
AAATCTTGAAGTCATGAAAGCTTCTGTTATTGATGGATTAACCCCTAGTA
AATCAATCAGTGGTTTAACAGGCGGTGATGCTGTCAAGATGGATCAATAT
TTACAATCAGGAAAAACTATTTCAGATACCACAATCCTAGCTGCCGTTAG
GAATGCTATGGCTGTTAATGAGTTAAATGCTAAGATGGGACTGGTCTGTG
CAACACCAACTGCAGGTAGTGCAGGATGTTTACCAGCTGTGATTTCTACA
GCCATTGAAAAGCTTAATTTAACAGAAGAAGAGCAACTTGTTTTCTATTT
ACAGCCGGCGCATTTGGTCTCGTCATTGGTAATAATGCCTCTATCTCAGG
TGCAGAAGGAGGTTGCCAAGCTGAAGTTGGGTCAGCTAGTGCTATGGCTG
CGGCTGCTTTAGTTATGGCTGCTGGAGGTACTCCTTTCCAAGCTAGCCAA
GCTATAGCATTTGTTATTAAAAATATGCTTGGACTTATCTGTGACCCTGT
TGCAGGTTTAGTTGAAGTCCCTTGTGTGAAGCGGAATGCTCTTGGATCAA
GTTTTGCACTTGTTGCTGCTGATATGGCCTTGGCTGGTATTGAATCGCAA
ATTCCAGTAGATGAAGTTATTGATGCAATGTATCAAGTTGGATCAAGTTT
ACCGACTGCTTTTCGTGAGACTGCAGAAGGAGGACTTGCTGCCACGCCGA
CAGGAAGACGTTATAGTAAAGAAATTTTTGGGGAA

SEQ ID NO.26

MFYTIEELVEQANSQHKGNIAELMIQTEIEMTGRSREEIRYIMSRNLEVM
KASVIDGLTPSKSISGLTGGDAVKMDQYLQSGKTISDTTILAAVRNAMAV
NELNAKMGLVCATPTAGSAGCLPAVISTAIEKLNLTEEEQLDFLFTAGAF
GLVIGNNASISGAEGGCQAEVGSASAMAAAALVMAAGGTPFQASQAIAFV
IKNMLGLICDPVAGLVEVPCVKRNALGSSFALVAADMALAGIESQIPVDE
VIDAMYQVGSSLPTAFRETAEGGLAATPTGRRYSKEIFGE

The nucleotide and amino acid sequences of GBS 361 in Ref. 3 are SEQ ID 8769 and SEQ ID 8770. These sequences are set forth below as SEQ ID NOS 27 and 28:

SEQ ID NO.27

ATGAGCGTATATGTTAGTGGAATAGGAATTATTTCTTCTTTGGGAAAGAA
TTATAGCGAGCATAAACAGCATCTCTTCGACTTAAAAGAAGGAATTTCTA
AACATTTATATAAAAATCACGACTCTATTTTAGAATCTTATACAGGAAGC
ATAACTAGTGACCCAGAGGTTCCTGAGCAATACAAAGATGAGACACGTAA
TTTTAAATTTGCTTTTACCGCTTTTGAAGAGGCTCTTGCTTCTTCAGGTG
TTAATTTAAAAGCTTATCATAATATTGCTGTGTGTTTAGGGACCTCACTT
TGGGGAAAGAGTGCTGGTCAAAATGCCTTGTATCAATTTGAAGAAGGAGA
GCGTCAAGTAGATGCTAGTTTATTAGAAAAAGCATCTGTTTACCATATTG
CTGATGAATTGATGGCTTATCATGATATTGTGGGAGCTTCGTATGTTATT
TCAACCGCCTGTTCTGCAAGTAATAATGCCGTAATATTAGGAACACAATT
ACTTCAAGATGGCGATTGTGATTTAGCTATTTGTGGTGGCTGTGATGAGT
TAAGTGATATTTCTTTAGCAGGCTTCACATCACTAGGAGCTATTAATACA
GAAATGGCATGTCAGCCCTATTCTTCTGGAAAAGGAATCAATTTGGGTGA
GGGCGCTGGTTTTGTTGTTCTTGTCAAAGATCAGTCCTTAGCTAAATATG
GAAAAATTATCGGTGGTCTTATTACTTCAGATGGTTATCATATAACAGCA
CCTAAGCCAACAGGTGAAGGGGCGGCACAGATTGCAAAGCAGCTAGTGAC
TCAAAGCAGCTAGTGACTCAAGCAGGTATTGACTACAGTGAGATTGACTA
TATTAACGGTACAGGTACTCAAGCTAATGATAAAATGGAAAAAAATATGT
ATGGTAAGTTTTTCCCGACAACGACATTGATCAGCAGTACCAAGGGGCAA
ACGGGTCATACTCTAGGGGCTGCAGGTATTATCGAATTGATTAATTGTTT
AGCGGCAATAGAGGAACAGACTGTACCAGCAACTAAAAATGAGATTGGGA
TAGAAGGTTTTCCAGAAAATTTTGTCTATCATCAAAAGAGAGAATACCCA
ATAAGAAATGCTTTAAATTTTTCGTTTGCTTTTGGTGGAAATAATAGTGG
TGTCTTATTGTCATTTAGATTCACCTCTAGAAACATTACCTGCTAGAGAA
AATCTTAAAATGGCTATCTTATCATCTGTTGCTTCCATTTCTAAGAATGA
ATCACTTTCTATAACCTATGAAAAAGTTGCTAGTAATTTCAACGACTTTG
AAGCATTACGCTTTAAAGGGGCTAGACCACCCAAAACTGTCAACCCAGCA
CAATTTAGGAAAATGGATGATTTTTCCAAAATGGTTGCCGTAACAACAGC
TCAAGCACTAATAGAAAGCAATATTAATCTAAAAAAACAAGATACTTCAA
AAGTAGGAATTGTATTTACAACACTTTCTGGACCAGTTGAGGTTGTTGAA
GGTATTGAAAAGCAAATCACAACAGAAGGATATGCACATGTTTCTGCTTC
ACGATTCCCGTTTACAGTAATGAATGCAGCAGCTGGTATGCTTTCTATCA
TTTTTAAAATAACAGGTCCTTTATCTGTCATTTCGACAAATAGTGGAGCG
CTTGATGGTATACAATATGCCAAGGAAATGATGCGTAACGATAATCTAGA
CTATGTGATTCTTGTTTCTGCTAATCAGTGGACAGACATGAGTTTTATGT
GGTGGCAACAATTAAACTATGATAGTCAAATGTTTGTCGGTTCTGATTAT
TATTGTTCAGCACAAGTCCTCTCTCGTCAAGCATTGGATAATTCTCCTAT
AATATTAGGTAGTAAACAATTAAAATATAGCCATAAAACATTCACAGATG
TGATGACTATTTTTGATGCTGCGCTTCAAAATTTATTATCAGACTTAGGA
CTAACCATAAAAGATATCAAAGGTTTCGTTTGGAATGAGCGGAAGAAGGC
AGTTAGTTCAGATTATGATTTCTTAGCGAACTTGTCTGAGTATTATAATA
TGCCAAACCTTGCTTCTGGTCAGTTTGGATTTTCATCTAATGGTGCTGGT
GAAGAACTGGACTATACTGTTAATGAAAGTATAGAAAAGGGCTATTATTT
AGTCCTATCTTATTCGATCTTCGGTGGTATCTCTTTTGCTATTATTGAAA
AAAGG

SEQ ID NO.28

MSVYVSGAGAASSLGKNYSEHKQHLFDLKEGISKHLYKNDSILESYTGSI
TSDPEVPEQYKDETRNFKFAFTAFEEALASSGVNLKAYHNIAVCLGTSLG
GKSAGQNALYQFEEGERQVDASLLEKASVYHIADELMAYHDIVGASYVIS
TACSASNNAVILGTQLLQDGDCDLAICGGCDELSDISLAGFTSLGAINTE
MACQPYSSGKGINLGEGAGFVVLVKDQSLAKYGKIIGGLITSDGYHITAP
KPTGEGAAQIAKQLVTQAGIDYSEIDYINGHGTGTQANDKMEKNMYGKFF
PTTTLISSTKGQTGHTLGAAGIIELINCLAAIEEQTVPATKNEIGIEGFP
ENFVYHQKREYPIRNALNFSFAFGGNNSGVLLSSLDSPLETLPARENLKM
AILSSVASISKNESLSITYEKVASNFNDFEALRFKGARPPKTVNPAQFRK
MDDFSKMVAVTTAQALIESNINLKKQDTSKVGIVFTTLSGPVEVVEGIEK
QITTEGYAHVSASRFPFTVMNAAAGMLSIIFKITGPLSVISTNSGALDGI
QYAKEMMRNDNLDYVILVSANQWTDMSTMWWQQLNYDSQMFVGSDYCSAQ
VLSRQALDNSPIILGSKQLKYSHKTFTDVMTIFDAALQNLLSDLGLTIKD
IKGFVWNERKKAVSSDYDFLANLSEYYNMPNLASGQFGFSSNGAGEELDY
TVNESIEKGYYLVLSYSIFGGISFAIIEKR

The nucleotide and amino acid sequences of GBS 404 in Ref. 3 are SEQ ID 8799 and SEQ ID 8800. These sequences are set forth below as SEQ ID NOS 29 and 30:

SEQ ID NO.29

ATGAAAATAGATGACCTAAGAAAAAGCGACAATGTTGAAGATCGTCGCTC
CAGTAGCGGAGGTCATTCTCTAGCGGAGGAAGTGGATTACCGATTCTTCA
ACTTTTATTGCTGCGAGGGAGTTGGAAAACCAAGCTTGTGGTTTTAATCA
TCTTACTGCTACTTGGCGGAGGGGGACTAACCAGCATTTTTAATGACTCA
TCCTCACCTTCTAGTTACCAATCTCAGAATGTCTCACGTTCTGTTGATAA
TAGCGCAACGAGAGAACAAATCGATTTCGTTAATAAAGTCCTTGGCTCAA
CTGAGGATTTCTGGTCACAAGAATTCCAAACCCAAGGTTTTGGAAATTAT
AAGGAACCAAAACTTGTTCTTTACACCAATTCAATTCAAACAGGTTGTGG
TATAGGTGAATCTGCTTCAGGACCATTTTTATTGTTCAGCAGATAAAAAA
ATCTATCTTGATATTTCTTTTTACAATGAATTATCACATAAATATGGTGC
TACTGGTGATTTTGCTATGGCCTACGTCATCGCCCACGAAGTTGGTCACC
ACATTCAAACAGAGTTAGGCATTATGGATAAGTATAATAGAATGCGACAC
GGACTTACTAAGAAAGAAGCAAATGCTTTAAATGTTCGGCTAGAACTTCA
AGCAGATTATTATGCAGGGGTATGGGCTCACTACATCAGGGGAAAAAATC
TCTTAGAACAAGGAGACTTTGAAGAGGCCATGAATGCTGCCCACGCCGTC
GGAGACGATACCCTTCAGAAAGAAACCTACGGAAAATTAGTGCCTGATAG
CTTTACCCATGGAACAGCTGAACAACGCCAACGTTGGTTTAACAAAGGCT
TTCAATATGGTGACATCCAACACGGTGATACTTTCTCCGTAGAACATCTA

SEQ ID NO.30

MKIDDLRKSDNVEDRRSSSGGSFSSGGSGLPILQLLLLRGSWKTKLVVLI
ILLLLGGGGLTSIFNDSSSPSSYQSQNVSRSVDNSATREQIDFVNKVLGS
TEDFWSQEFQTQFGNYKEPKLVLYTNSIQTGCGIGESASGPFYCSADKKI
YLDISFYNELSHKYGATGDFAMAYVIAHEVGHHIQTELGIMDKYNRMRHG
LTKKEANALNVRLELQADYYAGVWAHYIRGKNLLEQGDFEEAMNAAHAVG
DDTLQKETYGKLVPDSFTHGTAEQRQRWFNKGFQYGDIQHGDTFSVEHL

The nucleotide and amino acid sequences of GBS 656 in Ref. 3 are SEQ ID 9323 and SEQ ID 9324. These sequences are set forth below as SEQ ID NOS 31 and 32:

SEQ ID NO.31

ATGAAAAGATTACATAAACTGTTTATAACCGTAATTGCTACATTAGGTAT
GTTGGGGGTAATGACCTTTGGTCTTCCAACGCAGCCGCAAAACGTAACGC
CGATAGTACATGCTGATGCTGTCAATTCATCTATACGAGCCAGGAATTTC
AAAATAATTTAAAAAATGCTATTGGTAACCTACCATTTCAATATGTTAAT
GGTATTTATGAATTAAATAATAATCAGACAAATTTAAATGCTGATGCAAT
GTTAAAGCGTATGTTCAAAATACAATTGACAATCAACAAAGACTATCAAC
TGCTAATGCAATGCAAGATAGAACCAGTCAATATCAAAATCGCAGAGATA
CCACTCTTCCCGATGCAAATTGGAAACCATTAGGTTGGCATCAAGTAGCT
ACAGACCATTATGGACATGCAGTCGACAAGGGGCATTTAATTGCCTATGC
TTTAGCTGGAAATTTCAAAGGTTGGGATGCTTCCGTGTCAAATCCTCAAA
ATGTTGTCACACAAACAGCTCATTCCAACCAATCAAATCAAAAAATCAAT
CGTGGACAAAATTATTATGAAAGCTTAGTTCGTAAGGCGGTTGACCAAAA
CAAACGTGTTCGTTACCGTGTAACTCCATTGTACCGTAATGATACTGATT
TAGTTCCATTTGCAATGCACCTAGAAGCTAAATCACAAGATGGCACATTA
GAATTTAATGTTGCTATTCCAAACACACAAGCATCATACACTATGGATTA
TGCAACAGGAGAAATAACACTAAAT

SEQ ID NO.32

MKRLHKLFITVIATLGMLGVMTFGLPTQPQNVTPIVHADVNSSVDTSQEF
QNNLKNAGNLPFQYVNGIYELNNNQTNLNADVNVKAYVQNTIDNQQRLST
ANAMDRTIRQYQNRRDTTLPDANWKPLGWHQVATNDHYGHAVDKGHLIAY
ALAGNFKGWDASVSNPQNVVTQTAHSNQSNQKINRGQNYYESLVRKAVDQ
NKRVRYRVTPLYRNDTDLVPFAMHLEAKSQDGTLEFNVAIPNTQASYTMD
YATGEITLN

The nucleotide and amino acid sequences of GBS 690 in Ref. 3 are SEQ ID 9965 and SEQ ID 9966. These sequences are set forth as SEQ ID NOS 33 and 34 below:

SEQ ID NO.33

ATGAGTAAACGACAAAATTTAGGAATTAGTAAAAAGGAGCAATTATATCA
GGGCTCTCAGTGGCACTAATTGTAGTAATAGGTGGCTTTTTATGGGTACA
ATCTCAACCTAATAAGAGTGCAGTAAAAACTAACTACAAATTTTTAATGT
TAGAGAAGGAAGTGTTTCGTCCTCAACTCTTTTGACAGGAAAAGCTAAGG
CTAATCAAGAACAGTATGTGTATTTTGATGCTAATAAAGGTAATCGAGCA
ACTGTCACAGTTAAAGTGGGTGATAAAATCACAGCTGGTCAGCAGTTAGT
TCAATATATACAACAACTGCACAAGCAGCCTACGACACTGCTAATCGTCA
ATTAAATAAAGTAGCGCGTCAGATTAATAATCTAAAGACAACAGGAAGTC
TTCCAGCTATGGAATCAAGTGATCAATCTTCTTCATCATCACAAGGACAA
GGGACTCAATCGACTAGTGGTGCGACGAATCGTCTACAGCAAAATTATCA
AAGTCAAGCTAATGCTTCATACAACCAACAACTTCAAGATTTGAATGATG
CTTATGCAGATGCACAGGCAGAAGTAAATAAAGCACAAAAAGCATTGAAT
GATACTGTTATTACAAGTGACGTATCAGGGACAGTTGTTGAAGTTAATAG
TGATATTGATCCAGCTTCAAAAACTAGTCAAGTACTTGTCCATGTAGCAA
CTGAAGGTAAACTCCAAGTACAAGGAACGATGAGTGAGTATGATTTGGCT
AATGTTAAAAAAGACCAGGCTGTTAAAATAAAATCAAGGTCTATCCTGAC
AAGGAATGGGAAGGTAAAATTTCATATATCTCAAATTATCCAGAAGCAGA
AGCAAACAACAATGACTCTAATAACGGCTCTAGTGCTGTAAATTATAAAT
ATAAAGTAGATATTACTAGCCCTCTCGATGCATTAAAACAAGGTTTTACC
GTATCAGTTGAAGTAGTTAATGGAGATAAGCACCTTATTGTCCCTACAAG
TTCTGTGATAAACAAAGATAATAAACACTTTGTTTGGGTATACAATGATT
CTAATCGTAAAATTTCCAAAGTTGAAGTCAAAATTGGTAAAGCTGATGCT
AAGACACAAGAAATTTTATCAGGTTTGAAAGCAGGACAAATCGTGGTTAC
TAATCCAAGTAAAACCTTCAAGGATGGGCAAAAAATTGATAATATTGAAT
CAATCGATCTTAACTCTAATAAGAAATCAGAAGGTGAAA

SEQ ID NO.34

MSKRQNLGISKKGAIISGLSVALIVVIGGFLWVQSQPNKSAVKTNYKVFN
VREGSVSSSTLLTGKAKANQEQYVYFDANKGNRATVTVKVGDKITAGQQL
VQYDTTTAQAAYDTANRQLNKVARQINNLKTTGSLPAMESSDQSSSSSQG
QGTQSTSGATNRLQQNYQSQANASYNQQLQDLNDAYADAQAEVNKAQKAL
NDTVITSDVSGTVVEVNSDIDPASKTSQVLVHVATEGKLQVQGTMSEYDL
ANVKKDQAVKIKSKVYPDKEWEGKISYISNYPEAEANNNDSNNGSSAVNY
KYKVDITSPLDALKQGFTVSVEVNGDKHLIVPTSSVINKDNKHFVWVYND
SNRKISKVEVKIGKADAKTQEILSGLKAGQIVVTNPSKTFKDGQKIDNIE
SIDLNSNKKSEVK

The nucleotide and amino acid sequences of GBS 691 in Ref. 3 are SEQ ID 3691 and SEQ ID 3692. These sequences are set forth as SEQ ID NOS 35 and 36 below:

SEQ ID NO.35

ATGAAAAAAATTGGAATTATTGTCCTCACACTACGACCTTCTTTTTGGTA
TCTTGCGGACAACAAACTAAACAAGAAAGCACTAAAACAACTATTTCTAA
AATGCCTAAAATTGAAGGCTTCACCTATTATGGAAAAATTCCTGAAAATC
CGAAAAAAGTAATTAATTTTACATATTCTTACACTGGGTATTTATTAAAA
CTAGGTGTTAATGTTTCAAGTTACAGTTTAGACTTAGAAAAAGATAGCCC
CGTTTTTGGTAAACAACTGAAAGAAGCTAAAAAATTAACTGCTGATGATA
CAGAAGCTATTGCCGCACAAAAACCTGATTTAATCATGGTTTTCGATCAA
GATCCAAACATCAATACTCTGAAAAAAATTGCACCAACTTTAGTTATTAA
ATATGGTGCACAAAATTATTTAGATATGATGCCAGCCTTGGGGAAAGTAT
TCGGTAAAGAAAAAGAAGCTAATCAGTGGGTTAGCCAATGGAAAACTAAA
ACTCTCGCTGTCAAAAAAGATTTACACCATATCTTAAAGCCTAACACTAC
TTTTACTATTATGGATTTTTATGATAAAAATATCTATTTATATGGTAATA
ATTTTGGACGCGGTGGAGAACTAATCTATGATTCACTAGGTTATGCTGCC
CCAGAAAAAGTCAAAAAAGATGTCTTTAAAAAAGGGTGGTTTACCGTTTC
GCAAGAAGCAATCGGTGATTACGTTGGAGATTATGCCCTTGTTAATATAA
ACAAAACGACTAAAAAAGCAGCTTCATCACTTAAAGAAAGTGATGTCTGG
AAGAATTTACCAGCTGTCAAAAAAGGGCACATCATAGAAAGTAACTACGA
CGTGTTTTATTTCTCTGACCCTCTATCTTTAGAAGCTCAATTAAAATCAT
TTACAAAGGCTATCAAAGAAAATACAAAT

SEQ ID NO.36

MKKIGIIVLTLLTFFLVSCGQQTKQESTKTTISKMPKIEGFTYYGKIPEN
PKKVINFTYSYTGYLLKLGVNVSSYSLDLEKDSPVFGKQLKEAKKLTADD
TEAIAAQKPDLIMVFDQDPNINTLKKIAPTLVIKYGAQNYLDMMPALGKV
FGKEKEANQWVSQWKTKTLAVKKDLHHILKPNTTFTIMDFYDKNIYLYGN
NFGRGGELIYDSLGYAAPEKVKKDVFKKGWFTVSQEAIGDYVGDYALVNI
NKTTKKAASSLKESDVWKNLPAVKKGHIIESNYDVFYFSDPLSLEAQLKS
FTKAIKENTN

Other preferred polypeptide antigens include: GBS4 (SEQ ID 2 from Ref. 3); GBS22 (SEQ ID 8584 from Ref 3); and GBS85 (SEQ ID 216 from Ref. 3), including polypeptides having amino acid sequences with sequence identity thereto etc.

The polypeptide is preferably not a C protein (alpha or beta or epsilon) or a R protein (Rib).

The nucleotide and amino acid sequences of GBS 4 in Ref. 3 are SEQ ID 1 and SEQ ID 2.

These sequences are set forth below as SEQ ID NOS 37 and 38:

SEQ ID NO.37

ATGAAAGTGAAAAATAAGATTTTAACGATGGTAGCACTTACTGTCTTAAC
ATGTGCTACTTATTCATCAATCGGTTATGCTGATACAAGTGATAAGAATA
CTGACACGAGTGTCGTGACTACGACCTTATCTGAGGAGAAAAGATCAGAT
GAACTAGACCAGTCTAGTACTGGTTCTTCTTCTGAAAATGAATCGAGTTC
ATCAAGTGAACCAGAAACAAATCCGTCAACTAATCCACCTACAACAGAAC
CATCGCAACCCTCACCTAGTGAAGAGAACAAGCCTGATGGTAGAACGAAG
ACAGAAAGGCAATAATAAGGATATTTCTAGTGGAACAAAAGTATTAATTT
CAGAAGATAGTATTAAGAATTTTAGTAAAGCAAGTAGTGATCAAGAAGAA
GAAGTGGATCGCGATGAATCATCATCTTCAAAAGCAAATGATGGGAAAAA
AGGCCACAGTAAGCCTAAAAAGGAACTTCCTAAAACAGGAGATAGCCACT
CAGATACTGTAATAGCATCTACGGAGGGATTATTCTGTTATCATTAAGTT
TTTACAATAAGAAAATGAAACTTTAT

SEQ ID NO.38

MKVKNKILTMVALTVLTCATYSSIGYADTSDKNTDTSVVTTTLSEEKRSD
ELDQSSTGSSSENESSSSSEPETNPSTNPPTTEPSQPSPSEENKPDGRTK
TEIGNNKDISSGTKVLISEDSIKNFSKASSDQEEVDRDESSSSKANDGKK
GHSKPKKELPKTGDSHSDTVIASTGGIILLSLSFYNKKMKLY

The nucleotide and amino acid sequences of GBS 22 in Ref. 3 are SEQ 8583 and SEQ ID 8584. These sequences are set forth below as SEQ ID NOS 39 and 40:

SEQ ID NO.39

ATGAAAAGGATACGGAAAAGCCTTATTTTTGTTCTCGGAGTAGTTACCCT
AATTTGCTTATGTGCTTGTACTAAACAAAGCCAGCAAAAAAATGGCTTGT
CAGTAGTGACTAGCTTTTATCCAGTATATTCCATTACAAAAGCAGTTTCT
GGTGATTTGAATGATATTAAAATGATTCGATCACAGTCAGGTATTCATGG
TTTTGAACCCTCATCAAGTGATGTTGCTGCCATTTATGATGCTGATCTAT
TTCTATTTCTTTATCATTCGCACACACTAGAAGCTTGGGCGAGACGTTTG
GAACCTAGTTTGCATCACTCTAAAGTATCTGTAATTGAAGCTTCAAAAGG
TATGACTTTGGATAAAGTTCATGGCTTAGAAGATGTAGAGGCAGAAAAAG
GAGTAGATGAGTCAACCTTGTATGACCCTCACACTTGGAATGACCCTGTA
AAAGTATCTGAGGAAGCACAACTCATCGCTACACAATTAGCTAAAAAGGA
TCCTAAAAACGCTAAGGTTTATCAAAAAAATGCTGATCAATTTAGTGACA
AGGCAATGGCTATTGCAGAGAAGTATAAGCCAAAATTTAAAGCTGCAAAG
TCTAAATACTTTGTGACTTCACATACAGCATTCTCATACTTAGCTAAGCG
ATACGGATTGACTCAGTTAGGTATTGCAGGTGTCTCAACCGAGCAAGAAC
CTAGTGCTAAAAAATTAGCCGAAATTCAGGAGTTTGTGAAAACATATAAG
GTTAAGACTATTTTTGTTGAAGGAGTCTCACCTAAATTAGCTCAAGCAGT
AGCTTCAGCTACTCGAGTTAAAATTGCAAGTTTAAGTCCTTTARAAGCAG
TTCCCAAAAACAATAAAGATTACTTAGAAATTTGGAAACTAATCTTAAGG
TACTTGTCAAATCGTTAAATCAATAG

SEQ ID NO.40

MKRIRLSLIFVGVVTLICLCACTKQSQQKNGLSVVTSFYPVYSITKAVSG
DLNDIKMIRSQSGIHGFEPSSSDVAAIYDADLFLYHSHTLEAWARRLEPS
LHHSKVSVIEASKGMTLDKVHGLEDVEAEKGVDESTLYDPHTWNDPVKVS
EEAQLIATQLAKKDPKDPKNAKVYQKNDQFSDKAMAIAEKYKPKFKAAKS
KYFVTSHTAFSYLAKRYGLTQLGIAGVSTEQEPSAKKLAEIQEFVKTKYK
VKTIFVEEGVSPKLAQAVASATRVKIASLSPLXAVPRNNKDYLENLHTNL
KVLVKSLNQ

The nucleotide and amino acid sequences of GBS 85 in Ref. 3 are SEQ ID 215 and SEQ ID 216. These sequences are set forth below as SEQ ID NOS 41 and 42:

SEQ ID NO.41

ATGCCTAAGAAGAAATCAGATACCCCAGAAAAAGAAGAAGTTGTCTTAAC
GGAATGGCAAAAGCGTAACCTTGAATTTTTTAAAAAAACGCAAAGAAGAT
GAAGAAGAACAAAAACGTATTAACGAAAAATTACGCTTAGATAAAAGAAG
TAAATTAAATATTTCTTCTCCTGAAGAACCTCAAAATACTACTAAAATTA
AGAAGCTTCATTTTCCAAAGATTTCAAGACCTAAGATTGAAAAGAAACAG
AAAAAAGAAAAAATAGTCAACAGCTTAGCCAAAACTAATCGCATTAGAAC
TGCACCTATTTGTAGTAGCATTCCTAGTCATTTTAGTTTCCGTTTTCCTA
CTAACTCCTTTTAGTAAGCAAAAAACAATAACAGTTAGTGGAAATCAGCA
TACACCTGATGATATTTTGATAGAGAAAACGAATATTCAAAAAAACGATT
ATTTCTTTTCTTTAATTTTTAAACATAAAGCTATTGAACAACGTTTAGCT
GCAGAAGATGTATGGGTAAAAACAGCTCAGATGACTTATCAATTTCCCAA
TAAGTTTCATATTCAAGTTCAAGAAAATAAGATTATTGCATATGCACATA
CAAAGCAAGGATATCAACCTGTCTTGGAAACTGGAAAAAAGGCTGATCCT
GTAAATAGTTCAGAGCTACCAAAGCACTTCTTAACAATTAACCTTGATAA
GGAAGATAGTATTAAGCTATTAATTAAAGATTTAAAGGCTTTAGACCCTG
ATTTAATAAGTGAGATTCAGGTGATAAGTTTAGCTGATTCTAAAACGACA
CCTGACCTCCTGCTGTTAGATATGCACGATGGAAATAGTATTAGAATACC
ATTATCTAAATTTAAAGAAAGACTTCCTTTTTACAAACAAATTAAGAAGA
ACCTTAAGGAACCTTCTATTGTTGATATGGAAGTGGGAGTTTACACAACA
ACAAATACCATTGAATCAACCCCTGTTAAAGCAGAAGATACAAAAAATAA
ATAAATCAACTGATAAAACACAAACACAAAATGGTCAGGTTGCGGAAAAT
AGTCAAGGACAAACAAATAACTCAAATACTAATCAACAAGGACAACAGAT
AGCAACAGAGCAGGCACCTAACCCTCAAAATGTTAAT

SEQ ID NO.42

MPKKKSDTPEKEEVVLTEWQKRNLEFLKKRKEDEEEQKRINEKLRLDKRS
KLNISSPEEPQNTTKIKKLHFPKISRPKIEKKQKKEKIVNSLAKTNRIRT
APIFVVAFLVILVSVFLLTPFSKQKTITVSGNQHTPDDILIEKTNIQKND
YFFSLIFKHKAIEQRLAAEDVWVKTAQMTYQFPNKFHIQVQENKILAYAH
TKQGYQPVLETGKKADPVNSSELPKHFLTINLDKEDSIKLLIKDLKALDP
DLISEIQVISLADSKTTPKLLLLDMHDGNSIRIPLSKFKERLPFYKQIKK
NLKEPSIVDMEVGVYTTTNTIESTPVAEDTKNKSTDKTQTQNGQVAENSQ
GQTNNSNTNQQGQQIATEQAPNPQNVN

GBS polypeptides of the invention may be present in the composition as individual separate polypeptides. It is preferred, however, that two or more (i.e. 2, 3, 4, 5, 6, 7, 8, 9, 10, 11, 12, 13, 14, 15, 16, 17, 18, 19 or 20) of the antigens are expressed as a single polypeptide chain (a ‘hybrid’ polypeptide). Hybrid polypeptides offer two principal advantages: first, a polypeptide that may be unstable or poorly expressed on its own can be assisted by adding a suitable hybrid partner that overcomes the problem; second, commercial manufacture is simplified as only one expression and purification need be employed in order to produce two polypeptides which are both antigenically useful.

The hybrid polypeptide may comprise two or more polypeptide sequences from the first antigen group. Accordingly, the invention includes a composition comprising a first amino acid sequence and a second amino acid sequence, wherein said first and second amino acid sequences are selected from a GBS antigen or a fragment thereof. Preferably, the first and second amino acid sequences in the hybrid polypeptide comprise different epitopes.

The hybrid polypeptide may comprise one or more polypeptide sequences from different GBS serotypes. Accordingly, the invention includes a composition comprising a first amino acid sequence and a second amino acid sequence, said first amino acid sequence and said second amino acid sequence selected from a GBS serotype selected from the group consisting of serotypes Ia, Ib, Ia/c, II, III, IV, V, VI, VII and VIII. The first and second amino acid sequence may be from the same GBS serotype or they may be from different GBS serotypes. Preferably, the first and second amino acid sequence are selected a GBS serotype selected from the group consisting of serotypes II and V. Most preferably, at least one of the first and second amino acid sequences is from GBS serotype V. Preferably, the first and second amino acid sequences in the hybrid polypeptide comprise difference epitopes.

In one embodiment, the hybrid polypeptide comprises one or more GBS antigens from serotype V. Preferably, the hybrid polypeptide comprises a first amino acid sequence and a second amino acid sequence, said first amino acid sequence and said second amino acid sequence comprising a GBS antigen or a fragment thereof selected from the group consisting of GBS 80, GBS 91, GBS 104, GBS 147, GBS 173, GBS 276, GBS 305, GBS 313, GBS 322, GBS 328, GBS 330, GBS 338, GBS 358, GBS 361, GBS 404, GBS 656, GBS 690, and GBS 691. Preferably, the GBS antigen or fragment thereof is selected from the group consisting of GBS 80 and GBS 691. Preferably, the first and second amino acid sequences in the hybrid polypeptide comprise difference epitopes.

Hybrids consisting of amino acid sequences from two, three, four, five, six, seven, eight, nine, or ten GBS antigens are preferred. In particular, hybrids consisting of amino acid sequences from two, three, four, or five GBS antigens are preferred.

Different hybrid polypeptides may be mixed together in a single formulation. Within such combinations, a GBS antigen may be present in more than one hybrid polypeptide and/or as a non-hybrid polypeptide. It is preferred, however, that an antigen is present either as a hybrid or as a non-hybrid, but not as both.

Preferably, the GBS antigen in one of the hybrid polypeptides is GBS 80 or a fragment thereof. Accordingly, examples of two-antigen hybrids for use in the invention may comprise: (1) GBS 80 and GBS 91, (2) GBS 80 and GBS 104, (3) GBS 80 and GBS 147, (4) GBS 80 and GBS 173, (5) GBS 80 and GBS 276, (6) GBS 80 and GBS 305, (7) GBS 80 and GBS 313, (8) GBS 80 and GBS 322, (9) GBS 80 and GBS 328, (10) GBS 80 and GBS 330, (11) GBS 80 and GBS 338, (12) GBS 80 and GBS 358, (13) GBS 80 and GBS 361, (14) GBS 80 and GBS 404, (14) GBS 80 and GBS 404, (15) GBS 80 and GBS 656, (16) GBS 80 and GBS 690, and (17) GBS 80 and GBS 691. Preferably, a two-antigen hybrid for use in the invention comprises GBS 80 and GBS 691.

Hybrid polypeptides can be represented by the formula NH₂-A-{-X-L-}ₙ-B-COOH, wherein: X is an amino acid sequence of a GBS antigen or a fragment thereof; L is an optional linker amino acid sequence; A is an optional N-terminal amino acid sequence; B is an optional C-terminal amino acid sequence; and n is 2, 3, 4, 5, 6, 7, 8, 9, 10, 11, 12, 13, 14 or 15.

If a -X- moiety has a leader peptide sequence in its wild-type form, this may be included or omitted in the hybrid protein. In some embodiments, the leader peptides will be deleted except for that of the -X- moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {-X-L-}, linker amino acid sequence -L- may be present or absent. For instance, when −n=2 the hybrid may be NH₂-X₁-L₁-X₂-L₂-COOH, NH₂-X₁-X₂-COOH, NH₂-X₁-L₁-X₂-COOH, NH₂-X₁-X₂-L₂-COOH, etc. Linker amino acid sequence(s)-L- will typically be short (e.g. 20 or fewer amino acids i.e. 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ, where n =2, 3, 4, 5, 6, 7, 8, 9, 10 or more), and histidine tags (i.e. Hisₙ, where n=3, 4, 5, 6, 7, 8, 9, 10 or more). Other suitable linker amino acid sequences will be apparent to those skilled in the art. A useful linker is GSGGGG (SEQ ID 1), with the Gly-Ser dipeptide being formed from a BamHI restriction site, thus aiding cloning and manipulation, and the (Gly)₄ tetrapeptide being a typical poly-glycine linker. -A- is an optional N-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine.

-B- is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art.

Most preferably, n is 2 or 3.

The Saccharide Antigen

The saccharide antigen is generally the capsular polysaccharide of a GBS or a derivative thereof. Suitable derivatives include oligosaccharide (e.g. from 3 to 150, preferably 8 to 100, monosaccharide units) fragments of the polysaccharide (e.g. refs. 12 to 16), de-acetylated saccharides (Ref 16), N-acroylated saccharides (16), saccharides with terminal aldehyde groups, etc.

The saccharide is preferably conjugated to a carrier molecule to enhance immunogenicity (e.g. see refs. 4 to 23 etc.). In some embodiments of the invention the GBS saccharide is conjugated to a GBS protein as defined above, thereby giving a polypeptide/saccharide combination of the invention in a single molecule. In other embodiments the GBS saccharide is conjugated to a non-GBS protein, in which case the conjugate will be combined with a separate GBS protein to give a polypeptide/saccharide combination of the invention.

Non-GBS carrier polypeptides include tetanus toxoid, the N. meningitidis outer membrane protein (24), synthetic peptides (25,26), heat shock proteins (27,28), pertussis proteins (29,30), protein D from H. influenzae (31), cytokines (32), lymphokines (32), hormones (32), growth factors (32), toxin A or B from C. difficile (33), iron-uptake proteins (34) etc. Preferred carrier proteins are the CRM197 diphtheria toxoid (35) and tetanus toxoid.

The saccharide and polypeptide are joined covalently. This may involve a direct covalent bond between the saccharide and polypeptide, or indirect coupling via a linker or spacer may be used (e.g. via a B-propionamido linker (16), etc.). Any suitable conjugation chemistry may be used (e.g reductive amination (21) etc.). Linkage is preferably via a terminal saccharide in the polysaccharide.

A single carrier molecule may carry saccharide antigens of a single type (e.g. saccharides derived from a single GBS serotype) or may carry multiple different antigens (e.g. saccharides derived from multiple GBS serotypes, all conjugated to the same carrier).

The saccharides can, of course, be prepared by various means (e.g. purification of the saccharide from GBS, chemical synthesis, etc.), in various sizes (e.g. full-length, fragmented, etc.) and may be derivatised for linking to carriers. They are preferably prepared in substantially pure form (i.e. substantially free from other streptococcal saccharides) or substantially isolated form. Processes for preparing capsular polysaccharides from GBS are well known in the art (e.g. refs. 36 to 39) and processes for preparing oligosaccharides from polysaccharides are also known (e.g. hydrolysis, sonication, enzymatic treatment, treatment with a base followed by nitrosation, etc. (12 to 16)).

As an alternative to using a saccharide antigen in non-conjugated combinations, a peptide mimetic of the GBS capsular polysaccharide may be used (e.g. 40). Suitable peptides can be selected by techniques such as phage display using protective anti-saccharide antibodies. As a further alternative, an anti-idiotypic antibody may be used instead of a saccharide antigen (e.g. ref. 41).

Prime/Boost Schedules

Polypeptide/saccharide combinations of the invention may be given as single doses or as part of a prime/boost schedule. In a prime/boost schedule, the combinations may be used as the priming dose, the boosting dose(s), or both.

If a combination is used for both priming and boosting, it is preferred to use the same combination both times. If a combination is used for only one of priming and boosting, it is preferred that the other dose should use the polypeptide or saccharide on which the combination is based. Thus the invention provides a prime-boost schedule where either (i) one of the saccharide and polypeptide antigens is used for priming an immune response and a combination are used for boosting the response, or (ii) combined saccharide and polypeptide antigens are used for priming an immune response but only one is used for boosting the response.

Various timings for priming and boosting are suitable for use with the invention. In one embodiment, a priming dose is given to a child and a booster is given to a teenager (13-18 years) or young adult (19-25 years). In another embodiment, a priming dose is given to a teenager or young adult and a booster is given during pregnancy. In another embodiment, a priming dose is given to a female who intends to become pregnant and a booster is given during pregnancy.

Immunogenic Pharmaceutical Compositions

Polypeptide/saccharide combinations are formulated as immunogenic compositions, and more preferably as compositions suitable for use as a vaccine in humans (e.g. children or adults). Vaccines of the invention may either be prophylactic (i.e. to prevent infection) or therapeutic (i.e. to treat disease after infection), but will typically be prophylactic. Accordingly, the invention includes a method for the therapeutic or prophylactic treatment of GBS infection in an animal susceptible to GBS infection comprising administering to said animal a therapeutic or prophylactic amount of the immunogenic compositions of the invention.

The composition of the invention is preferably sterile.

The composition of the invention is preferably pyrogen-free.

The composition of the invention generally has a pH of between 6.0 and 7.0, more preferably to between 6.3 and 6.9 e.g. 6.6±0.2. The composition is preferably buffered at this pH.

Other components suitable for human administration are disclosed in reference 42.

Vaccines of the invention may be administered in conjunction with other immunoregulatory agents. In particular, compositions will usually include an adjuvant. Preferred further adjuvants include, but are not limited to, one or more of the following set forth below:

A. Mineral Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphoshpates, orthophosphates), sulphates, etc. {e.g. see chapters 8 & 9 of ref. 43}), or mixtures of different mineral compounds, with the compounds taking any suitable form (e.g. gel, crystalline, amorphous, etc.), and with adsorption being preferred. The mineral containing compositions may also be formulated as a particle of metal salt. See ref 44.

B. Oil-Emulsions

Oil-emulsion compositions suitable for use as adjuvants in the invention include squalene-water emulsions, such as MF59 (5% Squalene, 0.5% Tween 80, and 0.5% Span 85, formulated into submicron particles using a microfluidizer). See ref. 45.

Complete Freund's adjuvant (CFA) and incomplete Freund's adjuvant (IFA) may also be used as adjuvants in the invention.

C. Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-LC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO 96/33739).

Combinations of saponins and cholesterols can be used to form unique particles called Immunostimulating Complexs (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP 0 109 942, WO 96/11711 and WO 96/33739. Optionally, the ISCOMS may be devoid of additional detergent. See ref. 46.

A review of the development of saponin based adjuvants can be found at ref. 47.

C. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO 03/024480, WO 03/024481, and Refs. 48, 49, 50 and 51. Virosomes are discussed further in, for example, Ref. 52

D. Bacterial or Microbial Derivatives

Adjuvants suitable for use in the invention include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Ref. 53.

(2) Lipid A Derivatives

Lipid A derivatives include derivatives of lipid A from Escherichia coli such as OM-174. OM-174 is described for example in Ref. 54 and 55.

(3) Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See ref. 56, WO 02/26757 and WO 99/62923 for examples of possible analog substitutions. The adjuvant effect of CpG oligonucleotides is further discussed in Refs. 57, 58, WO 98/40100, U.S. Pat. No. 6,207,646, U.S. Pat. No. 6,239,116, and U.S. Pat. No. 6,429,199.

The CpG sequence may be directed to TLR9, such as the motif GTCGTT or TTCGTT. See ref. 59. The CpG sequence may be specific for inducing a Th1 immune response, such as a CpG-A ODN, or it may be more specific for inducing a B cell response, such a CpG-B ODN. CpG-A and CpG-B ODNs are discussed in refs. 60, 61 and WO 01/95935. Preferably, the CpG is a CpG-A ODN. Preferably, the CpG oligonucleotide is constructed so that the 5′ end is accessible for receptor recognition. Optionally, two CpG oligonucleotide sequences may be attached at their 3′ ends to form “immunomers”. See, for example, refs. 62, 63, 64 and WO 03/035836.

(4) ADP-Ribosylating Toxins and Detoxified Derivatives thereof.

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO 95/17211 and as parenteral adjuvants in WO 98/42375. Preferably, the adjuvant is a detoxified LT mutant such as LT-K63, LT-R72, and LTR192G. The use of ADP-ribosylating toxins and detoxified derivaties thereof, particularly LT-K63 and LT-R72, as adjuvants can be found in Refs. 65, 66, 67, 68, 69, 70, 71 and 72 each of which is specifically incorporated by reference herein in their entirety. Numerical reference for amino acid substitutions is preferably based on the alignments of the A and B subunits of ADP-ribosylating toxins set forth in Domenighini et al., Mol. Microbiol (1995) 15(6):1165-1167, specifically incorporated herein by reference in its entirety.

E. Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

F. Bioadhesives and Mucoadhesives

Bioadhesives and mucoadhesives may also be used as adjuvants in the invention. Suitable bioadhesives include esterified hyaluronic acid microspheres (Ref. 73) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention. E.g., ref. 74.

G. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

H. Liposomes

Examples of liposome formulations suitable for use as adjuvants are described in U.S. Pat. No. 6,090,406, U.S. Pat. No. 5,916,588, and EP 0 626 169.

I. Polyoxyethylene Ether and Polyoxyethylene Ester Formulations

Adjuvants suitable for use in the invention include polyoxyethylene ethers and polyoxyethylene esters. Ref 75. Such formulations further include polyoxyethylene sorbitan ester surfactants in combination with an octoxynol (Ref. 76) as well as polyoxyethylene alkyl ethers or ester surfactants in combination with at least one additional non-ionic surfactant such as an octoxynol (Ref 77).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

J. Polyphosohazene (PCPP)

PCPP formulations are described, for example, in Ref. 78 and 79.

K. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

L. Imidazoguinolone Compounds.

Examples of imidazoquinolone compounds suitable for use adjuvants in the invention include Imiquamod and its homologues, described further in Ref. 80 and 81. The invention may also comprise combinations of aspects of one or more of the adjuvants identified above. For example, the following adjuvant compositions may be used in the invention:

(1) a saponin and an oil-in-water emulsion (ref. 82);

(2) a saponin (e.g., QS21)+a non-toxic LPS derivative (e.g., 3dMPL) (see WO 94/00153);

(3) a saponin (e.g., QS21)+a non-toxic LPS derivative (e.g., 3dMPL)+a cholesterol;

(4) a saponin (e.g. QS21)+3dMPL+IL-12 (optionally +a sterol) (Ref. 83); combinations of 3dMPL with, for example, QS21 and/or oil-in-water emulsions (Ref. 84);

(5) SAF, containing 10% Squalane, 0.4% Tween 80, 5% pluronic-block polymer L121, and thr-MDP, either microfluidized into a submicron emulsion or vortexed to generate a larger particle size emulsion.

(6) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and

(7) one or more mineral salts (such as an aluminum salt)+a non-toxic derivative of LPS (such as 3dPML).

Aluminium salts and MF59 are preferred adjuvants for parenteral immunisation. Mutant bacterial toxins are preferred mucosal adjuvants.

The composition may include an antibiotic.

GBS polypeptide(s) and saccharide(s) in the compositions of the invention will be present in ‘immunologically effective amounts’ i.e. the administration of that amount to an individual, either in a single dose or as part of a series, is effective for treatment or prevention of disease. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Typically, the compositions of the invention are prepared as injectables. Direct delivery of the compositions will generally be parenteral (e.g. by injection, either subcutaneously, intraperitoneally, intravenously or intramuscularly or delivered to the interstitial space of a tissue) or mucosal (e.g. oral or intranasal [85,86]). The compositions can also be administered into a lesion. The invention provides a syringe containing a composition of the invention.

Once formulated, the compositions of the invention can be administered directly to the subject. The subjects to be treated can be animals; in particular, human subjects can be treated. The vaccines are particularly useful for vaccinating children and teenagers, and more particularly females.

As well as GBS polypeptides and saccahrides, the composition of the invention may comprise further antigens. For example, the composition may comprise one or more of the following further antigens:
antigens from Helicobacter pylori such as CagA [87 to 90], VacA [91, 92], NAP [93, 94, 95], HopX [e.g. 96], HopY [e.g. 96] and/or urease.
a saccharide antigen from N. meningitidis serogroup A, C, W135 and/or Y, such as the oligosaccharide disclosed in ref 97 from serogroup C [see also ref 98] or the oligosaccharides of ref. 99.
a saccharide antigen from Streptococcus pneumoniae [e.g. 100, 101, 102].
an antigen from hepatitis A virus, such as inactivated virus [e.g. 103, 104].
an antigen from hepatitis B virus, such as the surface and/or core antigens [e.g. 104, 105].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 106 & 107].
a diphtheria antigen, such as a diphtheria toxoid [e.g. chapter 3 of ref 108] e.g. the CRM₁₉₇ mutant [e.g. 109].
a tetanus antigen, such as a tetanus toxoid [e.g. chapter 4 of ref. 128].
a saccharide antigen from Haemophilus influenzae B [e.g. 98].
an antigen from hepatitis C virus [e.g. 110].
an antigen from N. gonorrhoeae [e.g. 111, 112, 113, 114].
an antigen from Chlamydia pneumoniae [e.g. refs. 115 to 121].
an antigen from Chlamydia trachomatis [e.g. 122].
an antigen from Popphyromnonas gingivalis [e.g. 123].
polio antigen(s) [e.g. 124, 125] such as OPV or, preferably, IPV.
rabies antigen(s) [e.g. 126] such as lyophilised inactivated virus [e.g. 127, RabAvert™].
measles, mumps and/or rubella antigens [e.g. chapters 9, 10 & 11 of ref 128].
influenza antigen(s) [e.g, chapter 19 of ref. 128], such as the haemagglutinin and/or neuraminidase surface proteins.
an antigen from Moraxella catarrhalis [e.g. 129].
an antigen from Streptococcus pyogenes (group A streptococcus) [e.g. 3, 130, 131].
an antigen from Staphylococcus aureus [e.g. 132].
an antigen from Bacillus anthracis [e.g. 133, 134, 135].
an antigen from a virus in the flaviviridae family (genus flavivirus), such as from yellow fever virus, Japanese encephalitis virus, four serotypes of Dengue viruses, tick-borne encephalitis virus, West Nile virus.
a pestivirus antigen, such as from classical porcine fever virus, bovine viral diarrhoea virus, and/or border disease virus.
a parvovirus antigen e.g. from parvovirus B19.
a prion protein (e.g. the CJD prion protein)
an amyloid protein, such as a beta peptide [136]
a cancer antigen, such as those listed in Table 1 of ref. 137 or in tables 3 & 4 of ref. 138.

The composition may comprise one or more of these further antigens.

Toxic protein antigens may be detoxified where necessary (e.g. detoxification of pertussis toxin by chemical and/or genetic means [107]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred. Saccharide antigens are preferably in the form of conjugates. Carrier proteins for the conjugates are the same as those described above for GBS saccharide conjugation, with CRM197 being preferred.

Antigens in the composition will typically be present at a concentration of at least 1 μg/ml each. In general, the concentration of any given antigen will be sufficient to elicit an immune response against that antigen.

As an alternative to using protein antigens in the composition of the invention, nucleic acid encoding the antigen may be used. Protein components of the compositions of the invention may thus be replaced by nucleic acid (preferably DNA e.g. in the form of a plasmid) that encodes the protein.

Methods of Treating Patients

The invention provides polypeptide/saccharide combinations of the invention for use as medicaments. The medicament is preferably able to raise an immune response in a mammal (i.e. it is an immunogenic composition) and is more preferably a vaccine.

The invention also provides a method of raising an immune response in a patient, comprising administering to a patient a composition of the invention. The immune response is preferably protective against streptococcal disease, and may comprise a humoral immune response and/or a cellular immune response.

The invention also provides the use of polypeptide/saccharide combination of the invention in the manufacture of a medicament for raising an immune response in an patient. The medicament is preferably an immunogenic composition (e.g. a vaccine). The medicament is preferably for the prevention and/or treatment of a disease caused by GBS (e.g. meningitis, sepsis, chorioamnionitis).

The invention also provides for a kit comprising a first component comprising the immunogenic compositions of the invention. The kit may further include a second component comprising one or more of the following: instructions, syringe or other delivery device, adjuvant, or pharmaceutically acceptable formulating solution.

The invention also provides a delivery device pre-filled with the immunogenic compositions of the invention.

The invention also provides a method for raising an immune response in a mammal comprising the step of administering an effective amount of a composition of the invention. The immune response is preferably protective and preferably involves antibodies and/or cell-mediated immunity. The method may raise a booster response.

Process for Manufacturing

The invention provides a process for preparing a composition of the invention, comprising the step of mixing (i) one or more GBS polypeptide antigens with (ii) one or more GBS saccharide antigens.

The process may comprise the step of covalently linking the GBS polypeptide to the GBS saccharide in order to form a conjugate.

Definitions

The term “comprising” means “including” as well as “consisting ”e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

### MODES FOR CARRYING OUT THE INVENTION

GBS serotype III is grown in Todd-Hewitt broth as described in reference 36 and its capsular polysaccharide was purified. The polysaccharide is depolymerised, sized and purified as described in reference 14 to give oligosaccharide antigen. Similar procedures are used to prepare capsular polysaccharides from other GBS serotypes.

The oligosaccharide is either admixed with or covalently conjugated (directly or via a linker) to purified serotype V protein. Preferably, the protein comprises a GBS antigen or a fragment thereof selected from the group consisting of GBS 80, GBS 91, GBS 104, GBS 147, GBS 173, GBS 276, GBS 305, GBS 313, GBS 322, GBS 328, GBS 330, GBS 338, GBS 358, GBS 361, GBS 404, GBS 656, GBS 690, and GBS 691.

It will be understood that the invention has been described by way of example only and modifications may be made whilst remaining within the scope and spirit of the invention All documents cited herein are incorporated by reference in their entirety.

### REFERENCES (THE CONTENTS OF WHICH ARE HEREBY INCORPORATED BY REFERENCE)

[1] Schuchat (1999) Lancet 353(9146):516.
[2] Tettelin et al. (2002) Proc. Natl. Acad. Sci. USA, 10.1073/pnas. 182380799.
[3] International patent application WO02/34771.
[4] Kasper (1995) Proc Assoc Am Physicians 107:369-373.
[5] U.S. Pat. No. 6,426,074.
[6] European patent application EP-A-0866133.
[7] U.S. Pat. No. 5,820,860.
[8] U.S. Pat. No. 5,648,241.
[9] U.S. Pat. No. 5,834,444.
[10] International patent application WO91/04049.
[11] International patent application WO94/10317.
[12] International patent application WO87/06267.
[13] International patent application WO91/04335.
[14] U.S. Pat. No. 6,372,222.
[15] International patent application WO96/40795.
[16] WO00/10599.
[17] U.S. Pat. No. 5,302,386.
[18] International patent application WO94/06467.
[19] U.S. Pat. No. 5,993,825.
[20] U.S. Pat. No. 5,843,461.
[21] U.S. Pat. No. 5,795,580.
[22] International patent application WO98/09648.
[23] U.S. Pat. No. 6,280,738.
[24] EP-A-0372501
[25] EP-A-0378881
[26] EP-A-0427347
[27] WO93/17712
[28] WO94/03208
[29] WO98/58668
[30] EP-A-0471177
[31] WO00/56360
[32] WO91/01146
[33] WO00/61761
[34] WO01/72337
[35] Research Disclosure, 453077 (Jan. 2002)
[36] U.S. Pat. No. 4,207,414.
[37] U.S. Pat. No. 4,324,887.
[38] U.S. Pat. No. 4,367,221.
[39] U.S. Pat. No. 4,367,223.
[40] Pincus et al. (1998) J. Immunology 160:293-298.
[41] International patent application WO99/54457.
[42] Gennaro (2000) Remington: The Science and Practice of Pharmacy. 20th edition, ISBN: 0683306472.
43. Vaccine design:the subunit and adjuvant approach (1995) Powell & Newman. ISBN 0-306-44867-X.
44. WO00/23105.
45. WO90/14837.
46. WO00/07621.
47. Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.
48. Niikura et al., “Chimeric Recombinant Hepatitis E Virus-Like Particles as an Oral Vaccine Vehicle Presenting Foreign Epitopes”, Virology (2002) 293:273-280.
49. Lenz et al., “Papillomarivurs-Like Particles Induce Acute Activation of Dendritic Cells”, Journal of Immunology (2001) 5246-5355.
50. Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338.
51. Gerber et al., “Human Papillomavrisu Virus-Like Particles Are Efficient Oral Immunogens when Coadministered with Escherichia coli Heat-Labile Entertoxin Mutant R192G or CpG”, Journal of Virology (2001) 75(10):4752-4760.
52. Gluck et al., “New Technology Platforms in the Development of Vaccines for the Future”, Vaccine (2002) 20:B1-B16.
53. Johnson et al. (1999) Bioorg Med Chem Lett 9:2273-2278.
54. Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491.
55. Pajak, et al., “The Adjuvant OM-174 induces both the migration and maturation of murine dendritic cells in vivo”, Vaccine (2003) 21:836-842.
56. Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400.
57. Krieg, “CpG motifs: the active ingredient in bacterial extracts?”, Nature Medicine (2003) 9(7): 831-835.
58. McCluskie, et al., “Parenteral and mucosal prime-boost immunization strategies in mice with hepatitis B surface antigen and CpG DNA”, FEMS Immunology and Medical Microbiology (2002) 32:179-185.
59. Kandimalla, et al., “Toll-like receptor 9: modulation of recognition and cytokine induction by novel synthetic CpG DNAs”, Biochemical Society Transactions (2003) 31 (part 3): 654-658.
60. Blackwell, et al., “CpG-A-Induced Monocyte IFN-gamma-Inducible Protein-10 Production is Regulated by Plasmacytoid Dendritic Cell Derived IFN-alpha”, J. Immunol. (2003) 170(8):4061-4068.
61. Krieg, “From A to Z on CpG”, TRENDS in Immunology (2002) 23(2): 64-65.
62. Kandimalla, et al., “Secondary structures in CpG oligonucleotides affect immunostimulatory activity”, BBRC (2003) 306:948-953.
63. Kandimalla, et al., “Toll-like receptor 9: modulation of recognition and cytokine induction by novel synthetic GpG DNAs”, Biochemical Society Transactions (2003) 31(part 3):664-658.
64. Bhagat et al., “CpG penta- and hexadeoxyribonucleotides as potent immunomodulatory agents” BBRC (2003) 300:853-861.
65 Beignon, et al., “The LTR72 Mutant of Heat-Labile Enterotoxin of Escherichia coli Enahnces the Ability of Peptide Antigens to Elicit CD4+ T Cells and Secrete Gamma Interferon after Coapplication onto Bare Skin”, Infection and Immunity (2002) 70(6):3012-3019.
66 Pizza, et al., “Mucosal vaccines: non toxic derivatives of LT and CT as mucosal adjuvants”, Vaccine (2001) 19:2534-2541.
67 Pizza, et al., “LTK63 and LTR72, two mucosal adjuvants ready for clinical trials” Int J. Med. Microbiol (2000) 290(4-5):455-461.
68 Scharton-Kersten et al., “Transcutaneous Immunization with Bacterial ADP-Ribosylating Exotoxins, Subunits and Unrelated Adjuvants”, Infection and Immunity (2000) 68(9):5306-5313.
69 Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280.
70 Partidos et al., “Heat-labile enterotoxin of Escherichia coli and its site-directed mutant LTK63 enhance the proliferative and cytotoxic T-cell responses to intranasally co-immunized synthetic peptides”, Immunol. Lett. (1999) 67(3):209-216.
71 Peppoloni et al., “Mutants of the Escherichia coli heat-labile enterotoxin as safe and strong adjuvants for intranasal delivery of vaccines”, Vaccines (2003) 2(2):285-293.
72 Pine et al., (2002) “Intranasal immunization with influenza vaccine and a detoxified mutant of heat labile enterotoxin from Escherichia coli (LTK63)” J. Control Release (2002) 85(1-3):263-270.
73. Singh et al., (2001) J Cont. Rele. 70:267-276.
74. WO99/27960.
75. WO99/52549.
76. WO01/21207.
77. WO01/21152.
78. Andrianov et al., “Preparation of hydrogel microspheres by coacervation of aqueous polyphophazene solutions”, Biomaterials (1998) 19(1-3):109-115.
79. Payne et al., “Protein Release from Polyphosphazene Matrices”, Adv. Drug. Delivery Review (1998) 31(3):185-196.
80. Stanley, “Imiquimod and the imidazoquinolones: mechanism of action and therapeutic potential” Clin Exp Denmatol (2002) 27(7):571-577.
81. Jones, “Resiquimod 3M”, Curr Opin Investig Drugs (2003) 4(2):214-218.
82. WO99/11241.
83. WO98/57659.
84. European patent applications 0835318, 0735898 and 0761231.
[85] Bakcke et at. (2001) Infect. Immun. 69:5010-5015.
[86] Katial et al. (2002) Infect. Immun. 70:702-707.
[87] Covacci & Rappuoli (2000) J. Exp. Med. 19:587-592.
[88] WO93/18150.
[89] Covacci et al. (1993) Proc. Natl. Acad. Sci. USA 90: 5791-5795.
[90] Tummuru et al. (1994) Infect. Immun. 61:1799-1809.
[91] Marchetti et al. (1998) Vaccine 16:33-37.
[92] Telford et al. (1994) J. Exp. Med. 179:1653-1658.
[93] Evans et al. (1995) Gene 153:123-127.
[94] WO96/01272 & WO96/01273, especially SEQ ID NO:6.
[95] WO97/25429.
[96] WO98/04702.
[97] Costantino et al. (1992) Vaccine 10:691-698.
[98] Costantino et al. (1999) Vaccine 17:1251-1263.
[99] International patent application PCT/IB02/03191.
[100] Watson (2000) Pediatr Infect Dis J 19:331-332.
[101] Rubin (2000) Pediatr Clin North Am 47:269-285, v.
[102] Jedrzejas (2001) Microbiol Mol Biol Rev 65:187-207.
[103] Bell (2000) Pediatr Infect Dis J 19:1187-1188.
[104] Iwarson (1995) APMIS 103:321-326.
[105] Gerlich et al. (1990) Vaccine 8 Suppl:S63-68 & 79-80.
[106] Gustafsson et al. (1996) N. Engl. J. Med. 334:349-355.
[107] Rappuoli et al. (1991) TIBTECH 9:232-238.
[108] Vaccines (1988) eds. Plotkin & Mortimer. ISBN 0-7216-1946-0.
[109] Del Guidice et al. (1998) Molecular Aspects of Medicine 19:1-70.
[110] Hsu et al. (1999) Clin Liver Dis 3:901-915.
[111] International patent application WO99/24578.
[112] International patent application WO99/36544.
[113] International patent application WO99/57280.
[114] International patent application PCT/IB02/02069.
[115] International patent application WO02/02606.
[116] Kalman et al. (1999) Nature Genetics 21:385-389.
[117] Read et al. (2000) Nucleic Acids Res 28:1397-406.
[118] Shirai et al. (2000) J. Infect. Dis. 181 (Suppl 3):S524-S527.
[119] International patent application WO99/27105.
[120] International patent application WO00/27994.
[121] International patent application WO00/37494.
[122] International patent application WO99/28475.
[123] Ross et al. (2001) Vaccine 19:4135-4142.
[124] Sutter et al. (2000) Pediatr Clin North Am 47:287-308.
[125] Zinmmerman & Spann (1999) Am Fam Physician 59:113-118, 125-126.
[126] Dreesen (1997) Vaccine 15 Suppl:S2-6.
[127] MMWR Morb Mortal Wkly Rep 1998 Jan. 16;47(1):12, 19.
[128] Vaccines (1988) eds. Plotkin & Mortimer. ISBN 0-7216-1946-0.
[129] McMichael (2000) Vaccine 19 Suppl 1:S101-107.
[130] Dale (1999) Infect Dis Clin North Am 13:227-43, viii.
[131] Ferretti et al. (2001) PNAS USA 98: 4658-4663.
[132] Kuroda et al. (2001) Lancet 357(9264):1225-1240; see also pages 1218-1219.
[133] J Toxicol Clin Toxicol (2001) 39:85-100.
[134] Demnicheli et al. (1998) Vaccine 16:880-884.
[135] Stepanov et al. (1996) J Biotechnol 44:155-160.
[136] Ingram (2001) Trends Neurosci 24:305-307.
[137] Rosenberg (2001) Nature 411:380-384.
[138] Moingeon (2001) Vaccine 19:1305-1326.

1. An immunogenic composition comprising a GBS saccharide antigen and at least two GBS polypeptide antigens, wherein said GBS saccharide antigen comprises a saccharide selected from GBS serotype Ia, Ib, and III, and wherein said GBS polypeptide antigens comprise a combination of at least two polypeptide or fragments thereof selected from the antigen group consisting of GBS 80, GBS 91, GBS 104, GBS 147, GBS 173, GBS 276, GBS 305, GBS 313, GBS 322, GBS 328, GBS 330, GBS 338, GBS 358, GBS 361, GBS 404, GBS 656, GBS 690, and GBS 691.

2. The immunogenic composition of claim 1, wherein said GBS polypeptide antigens further comprise a GBS polypeptide or a fragment thereof of serogroup II.

3. The immunogenic composition of claim 1, wherein said GBS polypeptide antigen combination comprises GBS 80 or a fragment thereof.

4. The immunogenic composition of claim 3, wherein said GBS polypeptide antigens comprise a combination of two GBS antigens or fragments thereof selected from the group consisting of (1) GBS080 and GBS 91, (2) GBS 80 and GBS 104, (3) GBS 80 and GBS 147, (4) GBS 80 and GBS 173, (5) GBS 80 and GBS 276, (6) GBS 80 and GBS 305, (7) GBS 80 and GBS 313, (8) GBS 80 and GBS 322, (9) GBS 80 and GBS 328, (10) GBS 80 and GBS 330, (11) GBS 80 and GBS 338, (12) GBS 80 and GBS 358, (13) GBS 80 and GBS 361, (14) GBS 80 and GBS5404, (14) GBS 80 and GBS 404, (15) GBS 80 and GBS 656, (16) GBS 80 and GBS 690, and (17) GBS 80 and GBS 691.

5. The immunogenic composition of claim 4, wherein said combination is selected from the group consisting of (1) GBS 80 and GBS 338; (2) GBS 80 and GBS 361, (3) GBS 80 and GBS 305, (4) GBS 80 and GBS 328, (5) GBS 80 and GBS 690, (6) GBS 80 and GBS 691 and (7) GBS 80 and GBS 147.

6. The immunogenic composition of claim 4, wherein said combination comprises GBS 80 and GBS 691.

7. The immunogenic composition of claim 1, wherein said composition comprises a combination of at least three GBS polypeptide antigens.

8. The immunogenic composition of claim 7, wherein said combination comprises GBS 80 and GBS691.

9. The immunogenic composition of claim 7, wherein said combination comprises GBS 80.

10. The immunogenic composition of claim 1, wherein at least one GBS polypeptide antigen is covalently linked to the GBS saccharide antigen.

11. The immunogenic composition of claim 1, wherein said GBS saccharide antigen is covalently linked to a carrier protein.

12. The immunogenic composition of claim 11, wherein said carrier protein is selected from the group consisting of tetanus toxoid, diphtheria toxoid, N. meningitides outer membrane protein, heat shock protein, pertusis protein, protein D from H. influenzae, and toxin A or B from C. difficile.

13. The immunogenic composition of claim 12, wherein said carrier protein is selected from the group consisting of tetanus toxoid and diphtheria toxoid.

14. The immunogenic composition of claim 13, wherein said carrier protein is a diphtheria toxoid.

15. The immunogenic composition of claim 14, wherein said diphtheria toxoid is CRM197.

16. A method for the therapeutic or prophylactic treatment of GBS infection in an animal susceptible to GBS infection comprising administering to said animal a therapeutic or prophylactic amount of the immunogenic composition of claim 1.

17. A method for the manufacture of a medicament for raising an immune response against GBS comprising combining a GBS saccharide antigen and at least two GBS polypeptide antigens, wherein said GBS saccharide antigen comprises a saccharide selected from GBS serotype Ia, Ib, and III, and wherein said GBS polypeptide antigens comprise a combination of at least two polypeptide or fragments thereof selected from the antigen group consisting of GBS 80, GBS 91, GBS 104, GBS 147, GBS 173, GBS 276, GBS 305, GBS 313, GBS 322, GBS 328, GBS 330, GBS 338, GBS 358, GBS 361, GBS 404, GBS 656, GBS 690, and GBS 691.


SEQUENCE LISTING (42 sequences)

SEQ ID NO: 1 - DNA, 1662 bp, Streptococcus agalactiae
atgaaattatcgaagaagttattgttttcggctgctgttttaacaatggtggcggggtcaactgttgaaccagtagctcagtttgcgactggaatgagtattgtaagagctgcagaagtgtcacaagaacgcccagcgaaaacaacagtaaatatctataaattacaagctgatagttataaatcggaaattacttctaatggtggtatcgagaataaagacggcgaagtaatatctaactatgctaaacttggtgacaatgtaaaaggtttgcaaggtgtacagtttaaacgttataaagtcaagacggatatttctgttgatgaattgaaaaaattgacaacagttgaagcagcagatgcaaaagttggaacgattcttgaagaaggtgtcagtctacctcaaaaaactaatgctcaaggtttggtcgtcgatgctctggattcaaaaagtaatgtgagatacttgtatgtagaagatttaaagaattcaccttcaaacattaccaaagcttatgctgtaccgtttgtgttggaattaccagttgctaactctacaggtacaggtttcctttctgaaattaatatttaccctaaaaacgttgtaactgatgaaccaaaaacagataaagatgttaaaaaattaggtcaggacgatgcaggttatacgattggtgaagaattcaaatggttcttgaaatctacaatccctgccaatttaggtgactatgaaaaatttgaaattactgataaatttgcagatggcttgacttataaatctgttggaaaaatcaagattggttcgaaaacactgaatagagatgagcactacactattgatgaaccaacagttgataaccaaaatacattaaaaattacgtttaaaccagagaaatttaaagaaattgctgagctacttaaaggaatgacccttgttaaaaatcaagatgctcttgataaagctactgcaaatacagatgatgcggcatttttggaaattccagttgcatcaactattaatgaaaaagcagttttaggaaaagcaattgaaaatacttttgaacttcaatatgaccatactcctgataaagctgacaatccaaaaccatctaatcctccaagaaaaccagaagttcatactggtgggaaacgatttgtaaagaaagactcaacagaaacacaaacactaggtggtgctgagtttgatttgttggcttctgatgggacagcagtaaaatggacagatgctcttattaaagcgaatactaataaaaactatattgctggagaagctgttactgggcaaccaatcaaattgaaatcacatacagacggtacgtttgagattaaaggtttggcttatgcagttgatgcgaatgcagagggtacagcagtaacttacaaattaaaagaaacaaaagcaccagaaggttatgtaatccctgataaagaaatcgagtttacagtatcacaaacatcttataatacaaaaccaactgacatcacggttgatagtgctgatgcaacacctgatacaattaaaaacaacaaacgtccttcaatccctaatactggtggtattggtacggctatctttgtcgctatcggtgctgcggtgatggcttttgctgttaaggggatgaagcgtcgtacaaaagataac

SEQ ID NO: 2 - protein, 554 aa, Streptococcus agalactiae
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 3 - DNA, 1629 bp, Streptococcus agalactiae
atgaaaaaaggacaagtaaatgatactaagcaatcttactctctacgtaaatataaatttggtttagcatcagtaattttagggtcattcataatggtcacaagtcctgtttttgcggatcaaactacatcggttcaagttaataatcagacaggcactagtgtggatgctaataattcttccaatgagacaagtgcgtcaagtgtgattacttccaataatgatagtgttcaagcgtctgataaagttgtaaatagtcaaaatacggcaacaaaggacattactactcctttagtagagacaaagccaatggtggaaaaaacattacctgaacaagggaattatgtttatagcaaagaaaccgaggtgaaaaatacaccttcaaaatcagccccagtagctttctatgcaaagaaaggtgataaagttttctatgaccaagtatttaataaagataatgtgaaatggatttcatataagtctttttgtggcgtacgtcgatacgcagctattgagtcactagatccatcaggaggttcagagactaaagcacctactcctgtaacaaattcaggaagcaataatcaagagaaaatagcaacgcaaggaaattatacattttcacataaagtagaagtaaaaaatgaagctaaggtagcgagtccaactcaatttacattggacaaaggagacagaattttttacgaccaaatactaactattgaaggaaatcagtggttatcttataaatcattcaatggtgttcgtcgttttgttttgctaggtaaagcatcttcagtagaaaaaactgaagataaagaaaaagtgtctcctcaaccacaagcccgtattactaaaactggtagactgactatttctaacgaaacaactacaggttttgatattttaattacgaatattaaagatgataacggtatcgctgctgttaaggtaccggtttggactgaacaaggagggcaagatgatattaaatggtatacagctgtaactactggggatggcaactacaaagtagctgtatcatttgctgaccataagaatgagaagggtctttataatattcatttatactaccaagaagctagtgggacacttgtaggtgtaacaggaactaaagtgacagtagctggaactaattcttctcaagaacctattgaaaatggtttagcaaagactggtgtttataatattatcggaagtactgaagtaaaaaatgaagctaaaatatcaagtcagacccaatttactttagaaaaaggtgacaaaataaattatgatcaagtattgacagcagatggttaccagtggatttcttacaaatcttatagtggtgttcgtcgctatattcctgtgaaaaagctaactacaagtagtgaaaaagcgaaagatgaggcgactaaaccgactagttatcccaacttacctaaaacaggtacctatacatttactaaaactgtagatgtgaaaagtcaacctaaagtatcaagtccagtggaatttaattttcaaaagggtgaaaaaatacattatgatcaagtgttagtagtagatggtcatcagtggatttcatacaagagttattccggtattcgtcgctatattgaaatt

SEQ ID NO: 4 - protein, 543 aa, Streptococcus agalactiae
MKKGQVNDTKQSYSLRKYKFGLASVILGSFIMVTSPVFADQTTSVQVNNQTGTSVDANNSSNETSASSVITSNNDSVQASDKVVNSQNTATKDITTPLVETKPMVEKTLPEQGNYVYSKETEVKNTPSKSAPVAFYAKKGDKVFYDQVFNKDNVKWISYKSFCGVRRYAAIESLDPSGGSETKAPTPVTNSGSNNQEKIATQGNYTFSHKVEVKNEAKVASPTQFTLDKGDRIFYDQILTIEGNQWLSYKSFNGVRRFVLLGKASSVEKTEDKEKVSPQPQARITKTGRLTISNETTTGFDILITNIKDDNGIAAVKVPVWTEQGGQDDIKWYTAVTTGDGNYKVAVSFADHKNEKGLYNIHLYYQEASGTLVGVTGTKVTVAGTNSSQEPIENGLAKTGVYNIIGSTEVKNEAKISSQTQFTLEKGDKINYDQVLTADGYQWISYKSYSGVRRYIPVKKLTTSSEKAKDEATKPTSYPNLPKTGTYTFTKTVDVKSQPKVSSPVEFNFQKGEKIHYDQVLVVDGHQWISYKSYSGIRRYIEI

SEQ ID NO: 5 - DNA, 2670 bp, Streptococcus agalactiae
atgaaaaagagacaaaaaatatggagagggttatcagttactttactaatcctgtcccaaattccatttggtatattggtacaaggtgaaacccaagataccaatcaagcacttggaaaagtaattgttaaaaaaacgggagacaatgctacaccattaggcaaagcgacttttgtgttaaaaaatgacaatgataagtcagaaacaagtcacgaaacggtagagggttctggagaagcaacctttgaaaacataaaacctggagactacacattaagagaagaaacagcaccaattggttataaaaaaactgataaaacctggaaagttaaagttgcagataacggagcaacaataatcgagggtatggatgcagataaagcagagaaacgaaaagaagttttgaatgcccaatatccaaaatcagctatttatgaggatacaaaagaaaattacccattagttaatgtagagggttccaaagttggtgaacaatacaaagcattgaatccaataaatggaaaagatggtcgaagagagattgctgaaggttggttatcaaaaaaaattacaggggtcaatgatctcgataagaataaatataaaattgaattaactgttgagggtaaaaccactgttgaaacgaaagaacttaatcaaccactagatgtcgttgtgctattagataattcaaatagtatgaataatgaaagagccaataattctcaaagagcattaaaagctggggaagcagttgaaaagctgattgataaaattacatcaaataaagacaatagagtagctcttgtgacatatgcctcaaccatttttgatggtactgaagcgaccgtatcaaagggagttgccgatcaaaatggtaaagcgctgaatgatagtgtatcatgggattatcataaaactacttttacagcaactacacataattacagttatttaaatttaacaaatgatgctaacgaagttaatattctaaagtcaagaattccaaaggaagcggagcatataaatggggatcgcacgctctatcaatttggtgcgacatttactcaaaaagctctaatgaaagcaaatgaaattttagagacacaaagttctaatgctagaaaaaaacttatttttcacgtaactgatggtgtccctacgatgtcttatgccataaattttaatccttatatatcaacatcttaccaaaaccagtttaattcttttttaaataaaataccagatagaagtggtattctccaagaggattttataatcaatggtgatgattatcaaatagtaaaaggagatggagagagttttaaactgttttcggatagaaaagttcctgttactggaggaacgacacaagcagcttatcgagtaccgcaaaatcaactctctgtaatgagtaatgagggatatgcaattaatagtggatatatttatctctattggagagattacaactgggtctatccatttgatcctaagacaaagaaagtttctgcaacgaaacaaatcaaaactcatggtgagccaacaacattatactttaatggaaatataagacctaaaggttatgacatttttactgttgggattggtgtaaacggagatcctggtgcaactcctcttgaagctgagaaatttatgcaatcaatatcaagtaaaacagaaaattatactaatgttgatgatacaaataaaatttatgatgagctaaataaatactttaaaacaattgttgaggaaaaacattctattgttgatggaaatgtgactgatcctatgggagagatgattgaattccaattaaaaaatggtcaaagttttacacatgatgattacgttttggttggaaatgatggcagtcaattaaaaaatggtgtggctcttggtggaccaaacagtgatgggggaattttaaaagatgttacagtgacttatgataagacatctcaaaccatcaaaatcaatcatttgaacttaggaagtggacaaaaagtagttcttacctatgatgtacgtttaaaagataactatataagtaacaaattttacaatacaaataatcgtacaacgctaagtccgaagagtgaaaaagaaccaaatactattcgtgatttcccaattcccaaaattcgtgatgttcgtgagtttccggtactaaccatcagtaatcagaagaaaatgggtgaggttgaatttattaaagttaataaagacaaacattcagaatcgcttttgggagctaagtttcaacttcagatagaaaaagatttttctgggtataagcaatttgttccagagggaagtgatgttacaacaaagaatgatggtaaaatttattttaaagcacttcaagatggtaactataaattatatgaaatttcaagtccagatggctatatagaggttaaaacgaaacctgttgtgacatttacaattcaaaatggagaagttacgaacctgaaagcagatccaaatgctaataaaaatcaaatcgggtatcttgaaggaaatggtaaacatcttattaccaacactcccaaacgcccaccaggtgtttttcctaaaacagggggaattggtacaattgtctatatattagttggttctacttttatgatacttaccatttgttctttccgtcgtaaacaattg

SEQ ID NO: 6 - protein, 890 aa, Streptococcus agalactiae
MKKRQKIWRGLSVTLLILSQIPFGILVQGETQDTNQALGKVIVKKTGDNATPLGKATFVLKNDNDKSETSHETVEGSGEATFENIKPGDYTLREETAPIGYKKTDKTWKVKVADNGATIIEGMDADKAEKRKEVLNAQYPKSAIYEDTKENYPLVNVEGSKVGEQYKALNPINGKDGRREIAEGWLSKKITGVNDLDKNKYKIELTVEGKTTVETKELNQPLDVVVLLDNSNSMNNERANNSQRALKAGEAVEKLIDKITSNKDNRVALVTYASTIFDGTEATVSKGVADQNGKALNDSVSWDYHKTTFTATTHNYSYLNLTNDANEVNILKSRIPKEAEHINGDRTLYQFGATFTQKALMKANEILETQSSNARKKLIFHVTDGVPTMSYAINFNPYISTSYQNQFNSFLNKIPDRSGILQEDFIINGDDYQIVKGDGESFKLFSDRKVPVTGGTTQAAYRVPQNQLSVMSNEGYAINSGYIYLYWRDYNWVYPFDPKTKKVSATKQIKTHGEPTTLYFNGNIRPKGYDIFTVGIGVNGDPGATPLEAEKFMQSISSKTENYTNVDDTNKIYDELNKYFKTIVEEKHSIVDGNVTDPMGEMIEFQLKNGQSFTHDDYVLVGNDGSQLKNGVALGGPNSDGGILKDVTVTYDKTSQTIKINHLNLGSGQKVVLTYDVRLKDNYISNKFYNTNNRTTLSPKSEKEPNTIRDFPIPKIRDVREFPVLTISNQKKMGEVEFIKVNKDKHSESLLGAKFQLQIEKDFSGYKQFVPEGSDVTTKNDGKIYFKALQDGNYKLYEISSPDGYIEVKTKPVVTFTIQNGEVTNLKADPNANKNQIGYLEGNGKHLITNTPKRPPGVFPKTGGIGTIVYILVGSTFMILTICSFRRKQL

SEQ ID NO: 7 - DNA, 3699 bp, Streptococcus agalactiae
gtggataaacatcactcaaaaaaggctattttaaagttaacacttataacaactagtattttattaatgcatagcaatcaagtgaatgcagaggagcaagaattaaaaaaccaagagcaatcacctgtaattgctaatgttgctcaacagccatcgccatcggtaactactaatactgttgaaaaaacatctgtaacagctgcttctgctagtaatacagcgaaagaaatgggtgatacatctgtaaaaaatgacaaaacagaagatgaattattagaagagttatctaaaaaccttgatacgtctaatttgggggctgatcttgaagaagaatatccctctaaaccagagacaaccaacaataaagaaagcaatgtagtaacaaatgcttcaactgcaatagcacagaaagttccctcagcatatgaagaggtgaagccagaaagcaagtcatcgcttgctgttcttgatacatctaaaataacaaaattacaagccataacccaaagaggaaagggaaatgtagtagctattattgatactggctttgatattaaccatgatatttttcgtttagatagcccaaaagatgataagcacagctttaaaactaagacagaatttgaggaattaaaagcaaaacataatatcacttatgggaaatgggttaacgataagattgtttttgcacataactacgccaacaatacagaaacggtggctgatattgcagcagctatgaaagatggttatggttcagaagcaaagaatatttcgcatggtacacacgttgctggtatttttgtaggtaatagtaaacgtccagcaatcaatggtcttcttttagaaggtgcagcgccaaatgctcaagtcttattaatgcgtattccagataaaattgattcggacaaatttggtgaagcatatgctaaagcaatcacagacgctgttaatctaggagcaaaaacgattaatatgagtattggaaaaacagctgattctttaattgctctcaatgataaagttaaattagcacttaaattagcttctgagaagggcgttgcagttgttgtggctgccggaaatgaaggcgcatttggtatggattatagcaaaccattatcaactaatcctgactacggtacggttaatagtccagctatttctgaagatactttgagtgttgctagctatgaatcacttaaaactatcagtgaggtcgttgaaacaactattgaaggtaagttagttaagttgccgattgtgacttctaaaccttttgacaaaggtaaggcctacgatgtggtttatgccaattatggtgcaaaaaaagactttgaaggtaaggactttaaaggtaagattgcattaattgagcgtggtggtggacttgattttatgactaaaatcactcatgctacaaatgcaggtgttgttggtatcgttatttttaacgatcaagaaaaacgtggaaattttctaattccttaccgtgaattacctgtggggattattagtaaagtagatggcgagcgtataaaaaatacttcaagtcagttaacatttaaccagagttttgaagtagttgatagccaaggtggtaatcgtatgctggaacaatcaagttggggcgtgacagctgaaggagcaatcaagcctgatgtaacagcttctggctttgaaatttattcttcaacctataataatcaataccaaacaatgtctggtacaagtatggcttcaccacatgttgcaggattaatgacaatgcttcaaagtcatttggctgagaaatataaagggatgaatttagattctaaaaaattgctagaattgtctaaaaacatcctcatgagctcagcaacagcattatatagtgaagaggataaggcgttttattcaccacgtcagcaaggtgcaggtgtagttgatgctgaaaaagctatccaagctcaatattatattactggaaacgatggcaaagctaaaattaatctcaaacgaatgggagataaatttgatatcacagttacaattcataaacttgtagaaggtgtcaaagaattgtattatcaagctaatgtagcaacagaacaagtaaataaaggtaaatttgcccttaaaccacaagccttgctagatactaattggcagaaagtaattcttcgtgataaagaaacacaagttcgatttactattgatgctagtcaatttagtcagaaattaaaagaacagatggcaaatggttatttcttagaaggttttgtacgttttaaagaagccaaggatagtaatcaggagttaatgagtattccttttgtaggatttaatggtgattttgcgaacttacaagcacttgaaacaccgatttataagacgctttctaaaggtagtttctactataaaccaaatgatacaactcataaagaccaattggagtacaatgaatcagctccttttgaaagcaacaactatactgccttgttaacacaatcagcgtcttggggctatgttgattatgtcaaaaatggtggggagttagaattagcaccggagagtccaaaaagaattattttaggaacttttgagaataaggttgaggataaaacaattcatcttttggaaagagatgcagcgaataatccatattttgccatttctccaaataaagatggaaatagggacgaaatcactccccaggcaactttcttaagaaatgttaaggatatttctgctcaagttctagatcaaaatggaaatgttatttggcaaagtaaggttttaccatcttatcgtaaaaatttccataataatccaaagcaaagtgatggtcattatcgtatggatgctcttcagtggagtggtttagataaggatggcaaagttgtagcagatggtttttatacttatcgcttacgttacacaccagtagcagaaggagcaaatagtcaggagtcagactttaaagtacaagtaagtactaagtcaccaaatcttccttcacgagctcagtttgatgaaactaatcgaacattaagcttagccatgcctaaggaaagtagttatgttcctacatatcgtttacaattagttttatctcatgttgtaaaagatgaagaatatggggatgagacttcttaccattatttccatatagatcaagaaggtaaagtgacacttcctaaaacggttaagataggagagagtgaggttgcggtagaccctaaggccttgacacttgttgtggaagataaagctggtaatttcgcaacggtaaaattgtctgatctcttgaataaggcagtagtatcagagaaagaaaacgctatagtaatttctaacagtttcaaatattttgataacttgaaaaaagaacctatgtttatttctaaaaaagaaaaagtagtaaacaagaatctagaagaaataatattagttaagccgcaaactacagttactactcaatcattgtctaaagaaataactaaatcaggaaatgagaaagtcctcacttctacaaacaataatagtagcagagtagctaagatcatatcacctaaacataacggggattctgttaaccataccttacctagtacatcagatagagcaacgaatggtctatttgttggtactttggcattgttatctagtttacttctttatttgaaacccaaaaagactaaaaataatagtaaa

SEQ ID NO: 8 - protein, 1233 aa, Streptococcus agalactiae
VDKHHSKKAILKLTLITTSILLMHSNQVNAEEQELKNQEQSPVIANVAQQPSPSVTTNTVEKTSVTAASASNTAKEMGDTSVKNDKTEDELLEELSKNLDTSNLGADLEEEYPSKPETTNNKESNVVTNASTAIAQKVPSAYEEVKPESKSSLAVLDTSKITKLQAITQRGKGNVVAIIDTGFDINHDIFRLDSPKDDKHSFKTKTEFEELKAKHNITYGKWVNDKIVFAHNYANNTETVADIAAAMKDGYGSEAKNISHGTHVAGIFVGNSKRPAINGLLLEGAAPNAQVLLMRIPDKIDSDKFGEAYAKAITDAVNLGAKTINMSIGKTADSLIALNDKVKLALKLASEKGVAVVVAAGNEGAFGMDYSKPLSTNPDYGTVNSPAISEDTLSVASYESLKTISEVVETTIEGKLVKLPIVTSKPFDKGKAYDVVYANYGAKKDFEGKDFKGKIALIERGGGLDFMTKITHATNAGVVGIVIFNDQEKRGNFLIPYRELPVGIISKVDGERIKNTSSQLTFNQSFEVVDSQGGNRMLEQSSWGVTAEGAIKPDVTASGFEIYSSTYNNQYQTMSGTSMASPHVAGLMTMLQSHLAEKYKGMNLDSKKLLELSKNILMSSATALYSEEDKAFYSPRQQGAGVVDAEKAIQAQYYITGNDGKAKINLKRMGDKFDITVTIHKLVEGVKELYYQANVATEQVNKGKFALKPQALLDTNWQKVILRDKETQVRFTIDASQFSQKLKEQMANGYFLEGFVRFKEAKDSNQELMSIPFVGFNGDFANLQALETPIYKTLSKGSFYYKPNDTTHKDQLEYNESAPFESNNYTALLTQSASWGYVDYVKNGGELELAPESPKRIILGTFENKVEDKTIHLLERDAANNPYFAISPNKDGNRDEITPQATFLRNVKDISAQVLDQNGNVIWQSKVLPSYRKNFHNNPKQSDGHYRMDALQWSGLDKDGKVVADGFYTYRLRYTPVAEGANSQESDFKVQVSTKSPNLPSRAQFDETNRTLSLAMPKESSYVPTYRLQLVLSHVVKDEEYGDETSYHYFHIDQEGKVTLPKTVKIGESEVAVDPKALTLVVEDKAGNFATVKLSDLLNKAVVSEKENAIVISNSFKYFDNLKKEPMFISKKEKVVNKNLEEIILVKPQTTVTTQSLSKEITKSGNEKVLTSTNNNSSRVAKIISPKHNGDSVNHTLPSTSDRATNGLFVGTLALLSSLLLYLKPKKTKNNSK

SEQ ID NO: 9 - DNA, 2040 bp, Streptococcus agalactiae
atgaaacgtaaatactttattcttaatacggtgacggttttaacgttagctgctgcaatgaatactagcagtatctatgctaatagtactgagacaagtgcttcagtagttcctactacaaatactatcgttcaaactaatgacagtaatcctaccgcaaaatttgtatcagaatcaggacaatctgtaataggtcaagtaaaaccagataattctgcggcgcttacaacagttgacacgcctcatcatatttcagctccagatgctttaaaaacaactcaatcaagtcctgtcgttgagagtacttctactaagttaactgaagagacttacaaacaaaaagatggtcaagatttagccaacatggtgagaagtggtcaagttactagtgaggaactcgttaatatggcatacgatattattgctaaagaaaacccatctttaaatgcagtcattactactagacgccaagaagctattgaagaggctagaaaacttaaagataccaatcagccgtttttaggtgttcccttgttagtcaaggggttagggcacagtattaaaggtggtgaaaccaataatggcttgatctatgcagatggaaaaattagcacatttgacagtagctatgtcaaaaaatataaagatttaggatttattattttaggacaaacgaactttccagagtatgggtggcgtaatataacagattctaaattatacggtctaacgcataatccttgggatcttgctcataatgctggtggctcttctggtggaagtgcagcagccattgctagcggaatgacgccaattgctagcggtagtgatgctggtggttctatccgtattccatcttcttggacgggcttggtaggtttaaaaccaacaagaggattggtgagtaatgaaaagccagattcgtatagtacagcagttcattttccattaactaagtcatctagagacgcagaaacattattaacttatctaaagaaaagcgatcaaacgctagtatcagttaatgatttaaaatctttaccaattgcttatactttgaaatcaccaatgggaacagaagttagtcaagatgctaaaaacgctattatggacaacgtcacattcttaagaaaacaaggattcaaagtaacagagatagacttaccaattgatggtagagcattaatgcgtgattattcaaccttggctattggcatgggaggagctttttcaacaattgaaaaagacttaaaaaaacatggttttactaaagaagacgttgatcctattacttgggcagttcatgttatttatcaaaattcagataaggctgaacttaagaaatctattatggaagcccaaaaacatatggatgattatcgtaaggcaatggagaagcttcacaagcaatttcctattttcttatcgccaacgaccgcaagtttagcccctctaaatacagatccatatgtaacagaggaagataaaagagcgatttataatatggaaaacttgagccaagaagaaagaattgctctctttaatcgccagtgggagcctatgttgcgtagaacaccttttacacaaattgctaatatgacaggactcccagctatcagtatcccgacttacttatctgagtctggtttacccatagggacgatgttaatggcaggtgcaaactatgatatggtattaattaaatttgcaactttctttgaaaaacatcatggttttaatgttaaatggcaaagaataatagataaagaagtgaaaccatctactggcctaatacagcctactaactccctctttaaagctcattcatcattagtaaatttagaagaaaattcacaagttactcaagtatctatctctaaaaaatggatgaaatcgtctgttaaaaataaaccatccgtaatggcatatcaaaaagcacttcctaaaacaggtgatacagaatcaagcctatctccagttttagtagtaacccttttattagcttgttttagctttgtaacaaaaaagaatcagaaaagt

SEQ ID NO: 10 - protein, 680 aa, Streptococcus agalactiae
MKRKYFILNTVTVLTLAAAMNTSSIYANSTETSASVVPTTNTIVQTNDSNPTAKFVSESGQSVIGQVKPDNSAALTTVDTPHHISAPDALKTTQSSPVVESTSTKLTEETYKQKDGQDLANMVRSGQVTSEELVNMAYDIIAKENPSLNAVITTRRQEAIEEARKLKDTNQPFLGVPLLVKGLGHSIKGGETNNGLIYADGKISTFDSSYVKKYKDLGFIILGQTNFPEYGWRNITDSKLYGLTHNPWDLAHNAGGSSGGSAAAIASGMTPIASGSDAGGSIRIPSSWTGLVGLKPTRGLVSNEKPDSYSTAVHFPLTKSSRDAETLLTYLKKSDQTLVSVNDLKSLPIAYTLKSPMGTEVSQDAKNAIMDNVTFLRKQGFKVTEIDLPIDGRALMRDYSTLAIGMGGAFSTIEKDLKKHGFTKEDVDPITWAVHVIYQNSDKAELKKSIMEAQKHMDDYRKAMEKLHKQFPIFLSPTTASLAPLNTDPYVTEEDKRAIYNMENLSQEERIALFNRQWEPMLRRTPFTQIANMTGLPAISIPTYLSESGLPIGTMLMAGANYDMVLIKFATFFEKHHGFNVKWQRIIDKEVKPSTGLIQPTNSLFKAHSSLVNLEENSQVTQVSISKKWMKSSVKNKPSVMAYQKALPKTGDTESSLSPVLVVTLLLACFSFVTKKNQKS

SEQ ID NO: 11 - DNA, 3402 bp, Streptococcus agalactiae
ttgcgtaaaaaacaaaaactaccatttgataaacttgccattgcgcttatatctacgagcatcttgctcaatgcacaatcagacattaaagcaaatactgtgacagaagacactcctgctaccgaacaagccgtagaacccccacaaccaatagcagtttctgaggaatcacgatcatcaaaggaaactaaaacctcacaaactcctagtgatgtaggagaaacagtagcagatgacgctaatgatctagcccctcaagctcctgctaaaactgctgatacaccagcaacctcaaaagcgactattagggatttgaacgacccttctcatgtcaaaaccctgcaggaaaaagcaggcaagggagctgggaccgttgttgcagtgattgatgctggttttgataaaaatcatgaagcgtggcgcttaacagacaaaactaaagcacgttaccaatcaaaagaaaatcttgaaaaagctaaaaaagagcacggtattacctatggcgagtgggtcaatgataaggttgcttattaccacgactatagtaaagatggtaaaaacgctgttgatcaagaacacggcacacacgtgtcagggatcttgtcaggaaatgctccatctgaaatgaaagaaccttaccgcctagaaggtgcgatgcctgaggctcaattgcttttgatgcgtgtcgaaattgtaaatggactagcagactatgctcgtaactacgctcaagctatcagagatgctgtcaacttgggagctaaggtgattaatatgagctttggtaatgctgcactagcttacgccaaccttccagacgaaaccaaaaaagcctttgactatgccaaatcaaaaggtgttagcattgtgacctcagctggtaatgatagtagctttgggggcaagccccgtctacctctagcagatcatcctgattatggggtggttgggacacctgcagcggcagattcaacattgacagttgcttcttacagcccagataaacagctcactgaaactgctacggtcaaaacagacgatcatcaagataaagaaatgcctgttatttcaacaaaccgttttgagccaaacaaggcttacgactatgcttatgctaatcgtggtacgaaagaggatgattttaaggatgtcgaaggtaagattgcccttattgaacgtggcgatattgatttcaaagataagattgcaaacgctaaaaaagctggtgctgtaggggtcttgatctatgacaatcaagacaagggcttcccgattgaattgccaaatgttgaccagatgcctgcggcctttatcagtcgaagagacggtctcttattaaaagacaatcccccaaaaaccattaccttcaatgcgacacctaaggtattgccaacagcaagtggcaccaaactaagccgcttctcaagctggggtctgacagctgacggcaatattaaaccggatattgcagcacccggccaagatattttgtcatcagtggctaacaacaagtatgccaaactttctggaactagtatgtctgcaccattggtagcgggtatcatgggactgttgcaaaagcaatatgagacacagtatcctgatatgacaccatcagagcgtcttgatttagctaagaaagtattgatgagctcagcaactgccctatatgatgaagatgaaaaagcttatttttctcctcgccaacagggagcaggagcagtcgatgctaaaaaagcttcagcagcaacgatgtatgtaacagataaggacaatacctcaagcaaggttcacctgaacaatgtttctgataaatttgaagtaacagtaacagttcacaacaaatctgataaacctcaagagttgtattaccaagtaactgttcaaacagataaagtagatggaaaacactttgccttggctcctaaagcattgtatgagacatcatggcaaaaaatcacaattccagccaatagcagcaaacaagtcaccgttccaatcgatgctagtcgatttagcaaggacttgcttgcccaaatgaaaaatggctatttcttagaaggttttgttcgtttcaaacaagatcctacaaaagaagagcttatgagcattccatatattggtttccgaggtgattttggcaatctgtcagccttagaaaaaccaatctatgatagcaaagacggtagcagctactatcatgaagcaaatagtgatgccaaagaccaattagatggtgatggattacagttttacgctctgaaaaataactttacagcacttaccacagagtctaacccatggacgattattaaagctgtcaaagaaggggttgaaaacatagaggatatcgaatcttcagagatcacagaaaccatttttgcaggtacttttgcaaaacaagacgatgatagccactactatatccaccgtcacgctaatggcaaaccatatgctgcgatctctccaaatggggacggtaacagagattatgtccaattccaaggtactttcttgcgtaatgctaaaaaccttgtggctgaagtcttggacaaagaaggaaatgttgtttggacaagtgaggtaaccgagcaagttgttaaaaactacaacaatgacttggcaagcacacttggttcaacccgttttgaaaaaacgcgttgggacggtaaagataaagacggcaaagttgttgctaacggaacctacacctatcgtgttcgctacacgccgattagctcaggtgcaaaagaacaacacactgattttgatgtgattgtagacaatacgacacctgaagtcgcaacatcggcaacattctcaacagaagatagtcgtttgacacttgcatctaaaccaaaaaccagccaaccggtttaccgtgagcgtattgcttacacttatatggatgaggatctgccaacaacagagtatatttctccaaatgaagatggtacctttactcttcctgaagaggctgaaacaatggaaggcgctactgttccattgaaaatgtcagactttacttatgttgttgaagatatggctggtaacatcacttatacaccagtgactaagctattggagggccactctaataagccagaacaagacggttcagatcaagcaccagacaagaaaccagaagctaaaccagaacaagacggttcaggtcaaacaccagataaaaaaaaagaaactaaaccagaaaaagatagttcaggtcaaacaccaggtaaaactcctcaaaaaggtcaatcttctcgtactctagagaaacgatcttctaagcgtgctttagctacaaaagcatcaacaagagatcagttaccaacgactaatgacaaggatacaaatcgtttacatctccttaagttagttatgaccactttcttcttggga

SEQ ID NO: 12 - protein, 1134 aa, Streptococcus agalactiae
MRKKQKLPFDKLAIALISTSILLNAQSDIKANTVTEDTPATEQAVEPPQPIAVSEESRSSKETKTSQTPSDVGETVADDANDLAPQAPAKTADTPATSKATIRDLNDPSHVKTLQEKAGKGAGTVVAVIDAGFDKNHEAWRLTDKTKARYQSKENLEKAKKEHGITYGEWVNDKVAYYHDYSKDGKNAVDQEHGTHVSGILSGNAPSEMKEPYRLEGAMPEAQLLLMRVEIVNGLADYARNYAQAIRDAVNLGAKVINMSFGNAALAYANLPDETKKAFDYAKSKGVSIVTSAGNDSSFGGKPRLPLADHPDYGVVGTPAAADSTLTVASYSPDKQLTETATVKTDDHQDKEMPVISTNRFEPNKAYDYAYANRGTKEDDFKDVEGKIALIERGDIDFKDKIANAKKAGAVGVLIYDNQDKGFPIELPNVDQMPAAFISRRDGLLLKDNPPKTITFNATPKVLPTASGTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQVTVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDSRLTLASKPKTSQPVYRERIAYTYMDEDLPTTEYISPNEDGTFTLPEEAETMEGATVPLKMSDFTYVVEDMAGNITYTPVTKLLEGHSNKPEQDGSDQAPDKKPEAKPEQDGSGQTPDKKKETKPEKDSSGQTPGKTPQKGQSSRTLEKRSSKRALATKASTRDQLPTTNDKDTNRLHLLKLVMTTFFLG

SEQ ID NO: 13 - DNA, 1365 bp, Streptococcus agalactiae
atgggacgagtaatgaaaacaataacaacatttgaaaataaaaaagttttagtccttggtttagcacgatctggagaagctgctgcacgtttgttagctaagttaggagcaatagtgacagttaatgatggcaaaccatttgatgaaaatccaacagcacagtctttgttggaagagggtattaaagtggtttgtggtagtcatcctttagaattgttagatgaggatttttgttacatgattaaaaatccaggaataccttataacaatcctatggtcaaaaaagcattagaaaaacaaatccctgttttgactgaagtggaattagcatacttagtttcagaatctcagctaataggtattacaggctctaacgggaaaacgacaacgacaacgatgattgcagaagtcttaaatgctggaggtcagagaggtttgttagctgggaatatcggctttcctgctagtgaagttgttcaggctgcgaatgataaagatactctagttatggaattatcaagttttcagctaatgggagttaaggaatttcgtcctcatattgcagtaattactaatttaatgccaactcatttagattatcatgggtcttttgaagattatgttgctgcaaaatggaatatccaaaatcaaatgtcttcatctgattttttggtacttaattttaatcaaggtatttctaaagagttagctaaaactactaaagcaacaatcgttcctttctctactacggaaaaagttgatggtgcttacgtacaagacaagcaacttttctataaaggggagaatattatgtcagtagatgacattggtgtcccaggaagccataacgtagagaatgctctagcaactattgcggttgctaaactggctggtatcagtaatcaagttattagagaaactttaagcaattttggaggtgttaaacaccgcttgcaatcactcggtaaggttcatggtattagtttctataacgacagcaagtcaactaatatattggcaactcaaaaagcattatctggctttgataatactaaagttatcctaattgcaggaggtcttgatcgcggtaatgagtttgatgaattgataccagatatcactggacttaaacatatggttgttttaggggaatcggcatctcgagtaaaacgtgctgcacaaaaagcaggagtaacttatagcgatgctttagatgttagagatgcggtacataaagcttatgaggtggcacaacagggcgatgttatcttgctaagtcctgcaaatgcatcatgggacatgtataagaatttcgaagtccgtggtgatgaattcattgatactttcgaaagtcttagaggagag

SEQ ID NO: 14 - protein, 455 aa, Streptococcus agalactiae
MGRVMKTITTFENKKVLVLGLARSGEAAARLLAKLGAIVTVNDGKPFDENPTAQSLLEEGIKVVCGSHPLELLDEDFCYMIKNPGIPYNNPMVKKALEKQIPVLTEVELAYLVSESQLIGITGSNGKTTTTTMIAEVLNAGGQRGLLAGNIGFPASEVVQAANDKDTLVMELSSFQLMGVKEFRPHIAVITNLMPTHLDYHGSFEDYVAAKWNIQNQMSSSDFLVLNFNQGISKELAKTTKATIVPFSTTEKVDGAYVQDKQLFYKGENIMSVDDIGVPGSHNVENALATIAVAKLAGISNQVIRETLSNFGGVKHRLQSLGKVHGISFYNDSKSTNILATQKALSGFDNTKVILIAGGLDRGNEFDELIPDITGLKHMVVLGESASRVKRAAQKAGVTYSDALDVRDAVHKAYEVAQQGDVILLSPANASWDMYKNFEVRGDEFIDTFESLRGE

SEQ ID NO: 15 - DNA, 1020 bp, Streptococcus agalactiae
atgaaacgtattgctgttttaactagtggtggtgacgcccctggtatgaacgctgctatccgtgcagttgttcgtaaagcaatttctgaaggtatggaagtttacggcatcaaccaaggttactatggtatggtgacaggggatattttccctttggatgctaattctgttggggatactatcaaccgtggaggaacgtttttacgttcagcacgttatcctgaatttgctgaacttgaaggtcagcttaaagggattgaacagcttaaaaaacacggtattgaaggtgtagtagttatcggtggtgatggttcttatcatggtgctatgcgtctaactgagcacggtttcccagctgttggtttgccgggtacaattgataacgatatcgttggcactgactatactattggttttgacacagcagttgcgacagcagttgagaatcttgaccgtcttcgtgatacatcagcaagtcataaccgtacttttgttgttgaggttatgggaagaaatgcaggagatatcgctctttggtcaggtatcgctgcaggtgcagatcaaattattgttcctgaagaagagttcaatattgatgaagttgtctcaaatgttagagctggctatgcagctggtaaacatcaccaaatcatcgtccttgcagaaggtgttatgagtggtgatgagtttgcaaaaacaatgaaagcagcaggagacgatagcgatcttcgtgtgacgaatttaggacatctgctccgtggtggtagtccgacggctcgtgatcgtgtcttagcatctcgtatgggagcgtacgctgttcaattgttgaaagaaggtcgtggtggtttagccgttggtgtccacaacgaagaaatggttgaaagtccaattttaggtttagcagaagaaggtgctttgttcagcttgactgatgaaggaaaaatcgttgttaataatccgcataaagcggaccttcgcttggcagcacttaatcgtgaccttgccaaccaaagtagtaaa

SEQ ID NO: 16 - protein, 340 aa, Streptococcus agalactiae
MKRIAVLTSGGDAPGMNAAIRAVVRKAISEGMEVYGINQGYYGMVTGDIFPLDANSVGDTINRGGTFLRSARYPEFAELEGQLKGIEQLKKHGIEGVVVIGGDGSYHGAMRLTEHGFPAVGLPGTIDNDIVGTDYTIGFDTAVATAVENLDRLRDTSASHNRTFVVEVMGRNAGDIALWSGIAAGADQIIVPEEEFNIDEVVSNVRAGYAAGKHHQIIVLAEGVMSGDEFAKTMKAAGDDSDLRVTNLGHLLRGGSPTARDRVLASRMGAYAVQLLKEGRGGLAVGVHNEEMVESPILGLAEEGALFSLTDEGKIVVNNPHKADLRLAALNRDLANQSSK

SEQ ID NO: 17 - DNA, 1376 bp, Streptococcus agalactiae
atgaataaaaaggtactattgacatcgacaatggcagcttcgctattatcagtcgcaagtgttcaagcacaagaaacagatacgacgtggacagcacgtactgtttcagaggtaaaggctgatttggtaaagcaagacaataaatcatcatatactgtgaaatatggtgatacactaagcgttatttcagaagcaatgtcaattgatatgaatgtcttagcaaaaataaataacattgcagatatcaatcttatttatcctgagacaacactgacagtaacttacgatcagaagagtcatactgccacttcaatgaaaatagaaacaccagcaacaaatgctgctggtcaaacaacagctactgtggatttgaaaaccaatcaagtttctgttgcagaccaaaaagtttctctcaatacaatttcggaaggtatgacaccagaagcagcaacaacgattgtttcgccaatgaagacatattcttctgcgccagctttgaaatcaaaagaagtattagcacaagagcaagctgttagtcaagcagcagctaatgaacaggtatcaccagctcctgtgaagtcgattacttcagaagttccagcagctaaagaggaagttaaaccaactcagacgtcagtcagtcagtcaacaacagtatcaccagcttctgttgccgctgaaacaccagctccagtagctaaagtagcaccggtaagaactgtagcagcccctagagtggcaagtgttaaagtagtcactcctaaagtagaaactggtgcatcaccagagcatgtatcagctccagcagttcctgtgactacgacttcaccagctacagacagtaagttacaagcgactgaagttaagagcgttccggtagcacaaaaagctccaacagcaacaccggtagcacaaccagcttcaacaacaaatgcagtagctgcacatcctgaaaatgcagggctccaacctcatgttgcagcttataaagaaaaagtagcgtcaacttatggagttaatgaattcagtacataccgtgcgggagatccaggtgatcatggtaaaggtttagcagttgactttattgtaggtactaatcaagcacttggtaataaagttgcacagtactctacacaaaatatggcagcaaataacatttcatatgttatctggcaacaaaagttttactcaaatacaaacagtatttatggacctgctaatacttggaatgcaatgccagatcgtggtggcgttactgccaaccactatgaccacgttcacgtatcatttaacaaataatataaaaaaggaagctatttggcttcttttttatatgccttgaatagactttcaaggttcttatataatttttatta

SEQ ID NO: 18 - protein, 432 aa, Streptococcus agalactiae
MNKKVLLTSTMAASLLSVASVQAQETDTTWTARTVSEVKADLVKQDNKSSYTVKYGDTLSVISEAMSIDMNVLAKINNIADINLIYPETTLTVTYDQKSHTATSMKIETPATNAAGQTTATVDLKTNQVSVADQKVSLNTISEGMTPEAATTIVSPMKTYSSAPALKSKEVLAQEQAVSQAAANEQVSPAPVKSITSEVPAAKEEVKPTQTSVSQSTTVSPASVAAETPAPVAKVAPVRTVAAPRVASVKVVTPKVETGASPEHVSAPAVPVTTTSPATDSKLQATEVKSVPVAQKAPTATPVAQPASTTNAVAAHPENAGLQPHVAAYKEKVASTYGVNEFSTYRAGDPGDHGKGLAVDFIVGTNQALGNKVAQYSTQNMAANNISYVIWQQKFYSNTNSIYGPANTWNAMPDRGGVTANHYDHVHVSFNK

SEQ ID NO: 19 - DNA, 2070 bp, Streptococcus agalactiae
atgaaaaagaaaattattttgaaaagtagtgttcttggtttagtcgctgggacttctattatgttctcaagcgtgttcgcggaccaagtcggtgtccaagttataggcgtcaatgactttcatggtgcacttgacaatactggaacagcaaatatgcctgatggaaaagttgctaatgctggtactgctgctcaattagatgcttatatggatgacgctcaaaaagatttcaaacaaactaaccctaatggtgaaagcattagggttcaagcaggcgatatggttggagcaagtccagccaactctgggcttcttcaagatgaaccaactgtcaaaaattttaatgcaatgaatgttgagtatggcacattgggtaaccatgaatttgatgaagggttggcagaatataatcgtatcgttactggtaaagcccctgctccagattctaatattaataatattacgaaatcatacccacatgaagctgcaaaacaagaaattgtagtggcaaatgttattgataaagttaacaaacaaattccttacaattggaagccttacgctattaaaaatattcctgtaaataacaaaagtgtgaacgttggctttatcgggattgtcaccaaagacatcccaaaccttgtcttacgtaaaaattatgaacaatatgaatttttagatgaagctgaaacaatcgttaaatacgccaaagaattacaagctaaaaatgtcaaagctattgtagttctcgcacatgtacctgcaacaagtaaaaatgatattgctgaaggtgaagcagcagaaatgatgaaaaaagtcaatcaactcttccctgaaaatagcgtagatattgtctttgctggacacaatcatcaatatacaaatggtcttgttggtaaaactcgtattgtacaagcgctctctcaaggaaaagcctatgctgatgtacgtggtgtcttagatactgatacacaagatttcattgagaccccttcagctaaagtaattgcagttgctcctggtaaaaaaacaggtagtgccgatattcaagccattgttgaccaagctaatactatcgttaaacaagtaacagaagctaaaattggtactgccgaggtaagtgtcatgattacgcgttctgttgatcaagataatgttagtccggtaggcagcctcatcacagaggctcaactagcaattgctcgaaaaagctggccagatatcgattttgccatgacaaataatggtggcattcgtgctgacttactcatcaaaccagatggaacaatcacctggggagctgcacaagcagttcaaccttttggtaatatcttacaagtcgtcgaaattactggtagagatctttataaagcactcaacgaacaatacgaccaaaaacaaaatttcttccttcaaatagctggtctgcgatacacttacacagataataaagagggcggggaagaaacaccatttaaagttgtaaaagcttataaatcaaatggtgaggaaatcaatcctgatgcaaaatacaaattagttatcaatgactttttattcggtggtggtgatggctttgcaagcttcagaaatgccaaacttctaggagccattaaccccgatacagaggtatttatggcctatatcactgatttagaaaaagctggtaaaaaagtgagcgttccaaataataaacctaaaatctatgtcactatgaagatggttaatgaaactattacacaaaatgatggtacacatagcattattaagaaactttatttagatcgacaaggaaatattgtagcacaagagattgtatcagacactttaaaccaaacaaaatcaaaatctacaaaaatcaaccctgtaactacaattcacaaaaaacaattacaccaatttacagctattaaccctatgagaaattatggcaaaccatcaaactccactactgtaaaatcaaaacaattaccaaaaacaaactctgaatatggacaatcattccttatgtctgtctttggtgttggacttataggaattgctttaaatacaaagaaaaaacatatgaaa

SEQ ID NO: 20 - protein, 690 aa, Streptococcus agalactiae
MKKKIILKSSVLGLVAGTSIMFSSVFADQVGVQVIGVNDFHGALDNTGTANMPDGKVANAGTAAQLDAYMDDAQKDFKQTNPNGESIRVQAGDMVGASPANSGLLQDEPTVKNFNAMNVEYGTLGNHEFDEGLAEYNRIVTGKAPAPDSNINNITKSYPHEAAKQEIVVANVIDKVNKQIPYNWKPYAIKNIPVNNKSVNVGFIGIVTKDIPNLVLRKNYEQYEFLDEAETIVKYAKELQAKNVKAIVVLAHVPATSKNDIAEGEAAEMMKKVNQLFPENSVDIVFAGHNHQYTNGLVGKTRIVQALSQGKAYADVRGVLDTDTQDFIETPSAKVIAVAPGKKTGSADIQAIVDQANTIVKQVTEAKIGTAEVSVMITRSVDQDNVSPVGSLITEAQLAIARKSWPDIDFAMTNNGGIRADLLIKPDGTITWGAAQAVQPFGNILQVVEITGRDLYKALNEQYDQKQNFFLQIAGLRYTYTDNKEGGEETPFKVVKAYKSNGEEINPDAKYKLVINDFLFGGGDGFASFRNAKLLGAINPDTEVFMAYITDLEKAGKKVSVPNNKPKIYVTMKMVNETITQNDGTHSIIKKLYLDRQGNIVAQEIVSDTLNQTKSKSTKINPVTTIHKKQLHQFTAINPMRNYGKPSNSTTVKSKQLPKTNSEYGQSFLMSVFGVGLIGIALNTKKKHMK

SEQ ID NO: 21 - DNA, 1500 bp, Streptococcus agalactiae
atgaataaacgcgtaaaaatcgttgcaacacttggtcctgcggttgaattccgtggtggtaagaagtttggtgagtctggatactggggtgaaagccttgacgtagaagcttcagcagaaaaaattgctcaattgattaaagaaggtgctaacgttttccgtttcaacttctcacatggagatcatgctgagcaaggagctcgtatggctactgttcgtaaagcagaagagattgcaggacaaaaagttggcttcctccttgatactaaaggacctgaaattcgtacagaactttttgaagatggtgcagatttccattcatatacaacaggtacaaaattacgtgttgctactaagcaaggtatcaaatcaactccagaagtgattgcattgaatgttgctggtggacttgacatctttgatgacgttgaagttggtaagcaaatccttgttgatgatggtaaactaggtcttactgtgtttgcaaaagataaagacactcgtgaatttgaagtagttgttgagaatgatggccttattggtaaacaaaaaggtgtaaacatcccttatactaaaattcctttcccagcacttgcagaacgcgataatgctgatatccgttttggacttgagcaaggacttaactttattgctatctcatttgtacgtactgctaaagatgttaatgaagttcgtgctatttgtgaagaaactggsmatggacacgttaagttgtttgctaaaattgaaaatcaacaaggtatcgataatattgatgagattatcgaagcagcagatggtattatgattgctcgtggtgatatgggtatcgaagttccatttgaaatggttccagtttaccaaaaaatgatcattactaaagttaatgcagctggtaaagcagttattacagcaacaaatatgcttgaaacaatgactgataaaccacgtgcgactcgttcagaagtatctgatgtcttcaatgctgttattgatggtactgatgctacaatgctttcaggtgagtcagctaatggtaaatacccagttgagtcagttcgtacaatggctactattgataaaaatgctcaaacattactcaatgagtatggtcgcttagactcatctgcattcccacgtaataacaaaactgatgttattgcatctgcggttaaagatgcaacacactcaatggatatcaaacttgttgtaacaattactgaaacaggtaatacagctcgtgccatttctaaattccgtccagatgcagacattttggctgttacatttgatgaaaaagtacaacgttcattgatgattaactggggtgttatccctgtccttgcagacaaaccagcatctacagatgatatgtttgaggttgcagaacgtgtagcacttgaagcaggatttgttgaatcaggcgataatatcgttatcgttgcaggtgttcctgtaggtacaggtggaactaacacaatgcgtgttcgtactgttaaa

SEQ ID NO: 22 - protein, 500 aa, Streptococcus agalactiae
  VARIANT (1)...(500): Xaa = Any Amino Acid
MNKRVKIVATLGPAVEFRGGKKFGESGYWGESLDVEASAEKIAQLIKEGANVFRFNFSHGDHAEQGARMATVRKAEEIAGQKVGFLLDTKGPEIRTELFEDGADFHSYTTGTKLRVATKQGIKSTPEVIALNVAGGLDIFDDVEVGKQILVDDGKLGLTVFAKDKDTREFEVVVENDGLIGKQKGVNIPYTKIPFPALAERDNADIRFGLEQGLNFIAISFVRTAKDVNEVRAICEETGXGHVKLFAKIENQQGIDNIDEIIEAADGIMIARGDMGIEVPFEMVPVYQKMIITKVNAAGKAVITATNMLETMTDKPRATRSEVSDVFNAVIDGTDATMLSGESANGKYPVESVRTMATIDKNAQTLLNEYGRLDSSAFPRNNKTDVIASAVKDATHSMDIKLVVTITETGNTARAISKFRPDADILAVTFDEKVQRSLMINWGVIPVLADKPASTDDMFEVAERVALEAGFVESGDNIVIVAGVPVGTGGTNTMRVRTVK

SEQ ID NO: 23 - DNA, 720 bp, Streptococcus agalactiae
ttgtctgctataatagacaaaaaggtggtgatatttatgtatttagcattaatcggtgatatcattaattcaaaacagatacttgaacgtgaaactttccaacagtcttttcagcaactaatgaccgaactatctgatgtatatggtgaagagctgatttctccattcactattacagctggtgatgaatttcaagctttattgaaaccatcaaaaaaggtatttcaaattattgaccatattcaactagctctaaaacctgttaatgtaaggttcggcctcggtacaggaaacattataacatccatcaattcaaatgaaagtatcggtgctgatggtcctgcctactggcatgctcgctcagctattaatcatatacatgataaaaatgattatggaacagttcaagtagctatttgccttgatgatgaagaccaaaaccttgaattaacactaaatagtctcatttcagctggtgattttatcaagtcaaaatggactacaaaccattttcaaatgcttgagcacttaatacttcaagataattatcaagaacaatttcaacatcaaaagttagcccaactggaaaatattgaacctagtgcgctgactaaacgccttaaagcaagcggtctgaagatttacttaagaacgagaacacaggcagccgatctattagttaaaagttgcactcaaactaaagggggaagctatgatttc

SEQ ID NO: 24 - protein, 240 aa, Streptococcus agalactiae
MSAIIDKKVVIFMYLALIGDIINSKQILERETFQQSFQQLMTELSDVYGEELISPFTITAGDEFQALLKPSKKVFQIIDHIQLALKPVNVRFGLGTGNIITSINSNESIGADGPAYWHARSAINHIHDKNDYGTVQVAICLDDEDQNLELTLNSLISAGDFIKSKWTTNHFQMLEHLILQDNYQEQFQHQKLAQLENIEPSALTKRLKASGLKIYLRTRTQAADLLVKSCTQTKGGSYDF

SEQ ID NO: 25 - DNA, 870 bp, Streptococcus agalactiae
atgttttatacaattgaagagctggtagagcaagctaatagccaacataagggtaacatagcagagctcatgatccaaacggaaattgaaatgactggtagaagtcgtgaagaaattcgttatattatgtcccgaaatcttgaagtcatgaaagcttctgttattgatggattaacccctagtaaatcaatcagtggtttaacaggcggtgatgctgtcaagatggatcaatatttacaatcaggaaaaactatttcagataccacaatcctagctgccgttaggaatgctatggctgttaatgagttaaatgctaagatgggactggtctgtgcaacaccaactgcaggtagtgcaggatgtttaccagctgtgatttctacagccattgaaaagcttaatttaacagaagaagagcaacttgattttctatttacagccggcgcatttggtctcgtcattggtaataatgcctctatctcaggtgcagaaggaggttgccaagctgaagttgggtcagctagtgctatggctgcggctgctttagttatggctgctggaggtactcctttccaagctagccaagctatagcatttgttattaaaaatatgcttggacttatctgtgaccctgttgcaggtttagttgaagtcccttgtgtgaagcggaatgctcttggatcaagttttgcacttgttgctgctgatatggccttggctggtattgaatcgcaaattccagtagatgaagttattgatgcaatgtatcaagttggatcaagtttaccgactgcttttcgtgagactgcagaaggaggacttgctgccacgccgacaggaagacgttatagtaaagaaatttttggggaa

SEQ ID NO: 26 - protein, 290 aa, Streptococcus agalactiae
MFYTIEELVEQANSQHKGNIAELMIQTEIEMTGRSREEIRYIMSRNLEVMKASVIDGLTPSKSISGLTGGDAVKMDQYLQSGKTISDTTILAAVRNAMAVNELNAKMGLVCATPTAGSAGCLPAVISTAIEKLNLTEEEQLDFLFTAGAFGLVIGNNASISGAEGGCQAEVGSASAMAAAALVMAAGGTPFQASQAIAFVIKNMLGLICDPVAGLVEVPCVKRNALGSSFALVAADMALAGIESQIPVDEVIDAMYQVGSSLPTAFRETAEGGLAATPTGRRYSKEIFGE

SEQ ID NO: 27 - DNA, 2193 bp, Streptococcus agalactiae
atgagcgtatatgttagtggaataggaattatttcttctttgggaaagaattatagcgagcataaacagcatctcttcgacttaaaagaaggaatttctaaacatttatataaaaatcacgactctattttagaatcttatacaggaagcataactagtgacccagaggttcctgagcaatacaaagatgagacacgtaattttaaatttgcttttaccgcttttgaagaggctcttgcttcttcaggtgttaatttaaaagcttatcataatattgctgtgtgtttagggacctcacttgggggaaagagtgctggtcaaaatgccttgtatcaatttgaagaaggagagcgtcaagtagatgctagtttattagaaaaagcatctgtttaccatattgctgatgaattgatggcttatcatgatattgtgggagcttcgtatgttatttcaaccgcctgttctgcaagtaataatgccgtaatattaggaacacaattacttcaagatggcgattgtgatttagctatttgtggtggctgtgatgagttaagtgatatttctttagcaggcttcacatcactaggagctattaatacagaaatggcatgtcagccctattcttctggaaaaggaatcaatttgggtgagggcgctggttttgttgttcttgtcaaagatcagtccttagctaaatatggaaaaattatcggtggtcttattacttcagatggttatcatataacagcacctaagccaacaggtgaaggggcggcacagattgcaaagcagctagtgactcaagcaggtattgactacagtgagattgactatattaacggtcacggtacaggtactcaagctaatgataaaatggaaaaaaatatgtatggtaagtttttcccgacaacgacattgatcagcagtaccaaggggcaaacgggtcatactctaggggctgcaggtattatcgaattgattaattgtttagcggcaatagaggaacagactgtaccagcaactaaaaatgagattgggatagaaggttttccagaaaattttgtctatcatcaaaagagagaatacccaataagaaatgctttaaatttttcgtttgcttttggtggaaataatagtggtgtcttattgtcatctttagattcacctctagaaacattacctgctagagaaaatcttaaaatggctatcttatcatctgttgcttccatttctaagaatgaatcactttctataacctatgaaaaagttgctagtaatttcaacgactttgaagcattacgctttaaaggggctagaccacccaaaactgtcaacccagcacaatttaggaaaatggatgatttttccaaaatggttgccgtaacaacagctcaagcactaatagaaagcaatattaatctaaaaaaacaagatacttcaaaagtaggaattgtatttacaacactttctggaccagttgaggttgttgaaggtattgaaaagcaaatcacaacagaaggatatgcacatgtttctgcttcacgattcccgtttacagtaatgaatgcagcagctggtatgctttctatcatttttaaaataacaggtcctttatctgtcatttcgacaaatagtggagcgcttgatggtatacaatatgccaaggaaatgatgcgtaacgataatctagactatgtgattcttgtttctgctaatcagtggacagacatgagttttatgtggtggcaacaattaaactatgatagtcaaatgtttgtcggttctgattattgttcagcacaagtcctctctcgtcaagcattggataattctcctataatattaggtagtaaacaattaaaatatagccataaaacattcacagatgtgatgactatttttgatgctgcgcttcaaaatttattatcagacttaggactaaccataaaagatatcaaaggtttcgtttggaatgagcggaagaaggcagttagttcagattatgatttcttagcgaacttgtctgagtattataatatgccaaaccttgcttctggtcagtttggattttcatctaatggtgctggtgaagaactggactatactgttaatgaaagtatagaaaagggctattatttagtcctatcttattcgatcttcggtggtatctcttttgctattattgaaaaaagg

SEQ ID NO: 28 - protein, 731 aa, Streptococcus agalactiae
MSVYVSGIGIISSLGKNYSEHKQHLFDLKEGISKHLYKNHDSILESYTGSITSDPEVPEQYKDETRNFKFAFTAFEEALASSGVNLKAYHNIAVCLGTSLGGKSAGQNALYQFEEGERQVDASLLEKASVYHIADELMAYHDIVGASYVISTACSASNNAVILGTQLLQDGDCDLAICGGCDELSDISLAGFTSLGAINTEMACQPYSSGKGINLGEGAGFVVLVKDQSLAKYGKIIGGLITSDGYHITAPKPTGEGAAQIAKQLVTQAGIDYSEIDYINGHGTGTQANDKMEKNMYGKFFPTTTLISSTKGQTGHTLGAAGIIELINCLAAIEEQTVPATKNEIGIEGFPENFVYHQKREYPIRNALNFSFAFGGNNSGVLLSSLDSPLETLPARENLKMAILSSVASISKNESLSITYEKVASNFNDFEALRFKGARPPKTVNPAQFRKMDDFSKMVAVTTAQALIESNINLKKQDTSKVGIVFTTLSGPVEVVEGIEKQITTEGYAHVSASRFPFTVMNAAAGMLSIIFKITGPLSVISTNSGALDGIQYAKEMMRNDNLDYVILVSANQWTDMSFMWWQQLNYDSQMFVGSDYCSAQVLSRQALDNSPIILGSKQLKYSHKTFTDVMTIFDAALQNLLSDLGLTIKDIKGFVWNERKKAVSSDYDFLANLSEYYNMPNLASGQFGFSSNGAGEELDYTVNESIEKGYYLVLSYSIFGGISFAIIEKR

SEQ ID NO: 29 - DNA, 900 bp, Streptococcus agalactiae
atgaaaatagatgacctaagaaaaagcgacaatgttgaagatcgtcgctccagtagcggaggttcattctctagcggaggaagtggattaccgattcttcaacttttattgctgcgagggagttggaaaaccaagcttgtggttttaatcatcttactgctacttggcggagggggactaaccagcatttttaatgactcatcctcaccttctagttaccaatctcagaatgtctcacgttctgttgataatagcgcaacgagagaacaaatcgatttcgttaataaagtccttggctcaactgaggatttctggtcacaagaattccaaacccaaggttttggaaattataaggaaccaaaacttgttctttacaccaattcaattcaaacaggttgtggtataggtgaatctgcttcaggaccattttattgttcagcagataaaaaaatctatcttgatatttctttttacaatgaattatcacataaatatggtgctactggtgattttgctatggcctacgtcatcgcccacgaagttggtcaccacattcaaacagagttaggcattatggataagtataatagaatgcgacacggacttactaagaaagaagcaaatgctttaaatgttcggctagaacttcaagcagattattatgcaggggtatgggctcactacatcaggggaaaaaatctcttagaacaaggagactttgaagaggccatgaatgctgcccacgccgtcggagacgatacccttcagaaagaaacctacggaaaattagtgcctgatagctttacccatggaacagctgaacaacgccaacgttggtttaacaaaggctttcaatatggtgacatccaacacggtgatactttctccgtagaacatcta

SEQ ID NO: 30 - protein, 300 aa, Streptococcus agalactiae
MKIDDLRKSDNVEDRRSSSGGSFSSGGSGLPILQLLLLRGSWKTKLVVLIILLLLGGGGLTSIFNDSSSPSSYQSQNVSRSVDNSATREQIDFVNKVLGSTEDFWSQEFQTQGFGNYKEPKLVLYTNSIQTGCGIGESASGPFYCSADKKIYLDISFYNELSHKYGATGDFAMAYVIAHEVGHHIQTELGIMDKYNRMRHGLTKKEANALNVRLELQADYYAGVWAHYIRGKNLLEQGDFEEAMNAAHAVGDDTLQKETYGKLVPDSFTHGTAEQRQRWFNKGFQYGDIQHGDTFSVEHL

SEQ ID NO: 31 - DNA, 783 bp, Streptococcus agalactiae
atgaaaagattacataaactgtttataaccgtaattgctacattaggtatgttgggggtaatgacctttggtcttccaacgcagccgcaaaacgtaacgccgatagtacatgctgatgtcaattcatctgttgatacgagccaggaatttcaaaataatttaaaaaatgctattggtaacctaccatttcaatatgttaatggtatttatgaattaaataataatcagacaaatttaaatgctgatgtcaatgttaaagcgtatgttcaaaatacaattgacaatcaacaaagactatcaactgctaatgcaatgcttgatagaaccattcgtcaatatcaaaatcgcagagataccactcttcccgatgcaaattggaaaccattaggttggcatcaagtagctactaatgaccattatggacatgcagtcgacaaggggcatttaattgcctatgctttagctggaaatttcaaaggttgggatgcttccgtgtcaaatcctcaaaatgttgtcacacaaacagctcattccaaccaatcaaatcaaaaaatcaatcgtggacaaaattattatgaaagcttagttcgtaaggcggttgaccaaaacaaacgtgttcgttaccgtgtaactccattgtaccgtaatgatactgatttagttccatttgcaatgcacctagaagctaaatcacaagatggcacattagaatttaatgttgctattccaaacacacaagcatcatacactatggattatgcaacaggagaaataacactaaat

SEQ ID NO: 32 - protein, 261 aa, Streptococcus agalactiae
MKRLHKLFITVIATLGMLGVMTFGLPTQPQNVTPIVHADVNSSVDTSQEFQNNLKNAIGNLPFQYVNGIYELNNNQTNLNADVNVKAYVQNTIDNQQRLSTANAMLDRTIRQYQNRRDTTLPDANWKPLGWHQVATNDHYGHAVDKGHLIAYALAGNFKGWDASVSNPQNVVTQTAHSNQSNQKINRGQNYYESLVRKAVDQNKRVRYRVTPLYRNDTDLVPFAMHLEAKSQDGTLEFNVAIPNTQASYTMDYATGEITLN

SEQ ID NO: 33 - DNA, 1242 bp, Streptococcus agalactiae
atgagtaaacgacaaaatttaggaattagtaaaaaaggagcaattatatcagggctctcagtggcactaattgtagtaataggtggctttttatgggtacaatctcaacctaataagagtgcagtaaaaactaactacaaagtttttaatgttagagaaggaagtgtttcgtcctcaactcttttgacaggaaaagctaaggctaatcaagaacagtatgtgtattttgatgctaataaaggtaatcgagcaactgtcacagttaaagtgggtgataaaatcacagctggtcagcagttagttcaatatgatacaacaactgcacaagcagcctacgacactgctaatcgtcaattaaataaagtagcgcgtcagattaataatctaaagacaacaggaagtcttccagctatggaatcaagtgatcaatcttcttcatcatcacaaggacaagggactcaatcgactagtggtgcgacgaatcgtctacagcaaaattatcaaagtcaagctaatgcttcatacaaccaacaacttcaagatttgaatgatgcttatgcagatgcacaggcagaagtaaataaagcacaaaaagcattgaatgatactgttattacaagtgacgtatcagggacagttgttgaagttaatagtgatattgatccagcttcaaaaactagtcaagtacttgtccatgtagcaactgaaggtaaactccaagtacaaggaacgatgagtgagtatgatttggctaatgttaaaaaagaccaggctgttaaaataaaatctaaggtctatcctgacaaggaatgggaaggtaaaatttcatatatctcaaattatccagaagcagaagcaaacaacaatgactctaataacggctctagtgctgtaaattataaatataaagtagatattactagccctctcgatgcattaaaacaaggttttaccgtatcagttgaagtagttaatggagataagcaccttattgtccctacaagttctgtgataaacaaagataataaacactttgtttgggtatacaatgattctaatcgtaaaatttccaaagttgaagtcaaaattggtaaagctgatgctaagacacaagaaattttatcaggtttgaaagcaggacaaatcgtggttactaatccaagtaaaaccttcaaggatgggcaaaaaattgataatattgaatcaatcgatcttaactctaataagaaatcagaggtgaaa

SEQ ID NO: 34 - protein, 414 aa, Streptococcus agalactiae
MSKRQNLGISKKGAIISGLSVALIVVIGGFLWVQSQPNKSAVKTNYKVFNVREGSVSSSTLLTGKAKANQEQYVYFDANKGNRATVTVKVGDKITAGQQLVQYDTTTAQAAYDTANRQLNKVARQINNLKTTGSLPAMESSDQSSSSSQGQGTQSTSGATNRLQQNYQSQANASYNQQLQDLNDAYADAQAEVNKAQKALNDTVITSDVSGTVVEVNSDIDPASKTSQVLVHVATEGKLQVQGTMSEYDLANVKKDQAVKIKSKVYPDKEWEGKISYISNYPEAEANNNDSNNGSSAVNYKYKVDITSPLDALKQGFTVSVEVVNGDKHLIVPTSSVINKDNKHFVWVYNDSNRKISKVEVKIGKADAKTQEILSGLKAGQIVVTNPSKTFKDGQKIDNIESIDLNSNKKSEVK

SEQ ID NO: 35 - DNA, 930 bp, Streptococcus agalactiae
atgaaaaaaattggaattattgtcctcacactactgaccttctttttggtatcttgcggacaacaaactaaacaagaaagcactaaaacaactatttctaaaatgcctaaaattgaaggcttcacctattatggaaaaattcctgaaaatccgaaaaaagtaattaattttacatattcttacactgggtatttattaaaactaggtgttaatgtttcaagttacagtttagacttagaaaaagatagccccgtttttggtaaacaactgaaagaagctaaaaaattaactgctgatgatacagaagctattgccgcacaaaaacctgatttaatcatggttttcgatcaagatccaaacatcaatactctgaaaaaaattgcaccaactttagttattaaatatggtgcacaaaattatttagatatgatgccagccttggggaaagtattcggtaaagaaaaagaagctaatcagtgggttagccaatggaaaactaaaactctcgctgtcaaaaaagatttacaccatatcttaaagcctaacactacttttactattatggatttttatgataaaaatatctatttatatggtaataattttggacgcggtggagaactaatctatgattcactaggttatgctgccccagaaaaagtcaaaaaagatgtctttaaaaaagggtggtttaccgtttcgcaagaagcaatcggtgattacgttggagattatgcccttgttaatataaacaaaacgactaaaaaagcagcttcatcacttaaagaaagtgatgtctggaagaatttaccagctgtcaaaaaagggcacatcatagaaagtaactacgacgtgttttatttctctgaccctctatctttagaagctcaattaaaatcatttacaaaggctatcaaagaaaatacaaat

SEQ ID NO: 36 - protein, 310 aa, Streptococcus agalactiae
MKKIGIIVLTLLTFFLVSCGQQTKQESTKTTISKMPKIEGFTYYGKIPENPKKVINFTYSYTGYLLKLGVNVSSYSLDLEKDSPVFGKQLKEAKKLTADDTEAIAAQKPDLIMVFDQDPNINTLKKIAPTLVIKYGAQNYLDMMPALGKVFGKEKEANQWVSQWKTKTLAVKKDLHHILKPNTTFTIMDFYDKNIYLYGNNFGRGGELIYDSLGYAAPEKVKKDVFKKGWFTVSQEAIGDYVGDYALVNINKTTKKAASSLKESDVWKNLPAVKKGHIIESNYDVFYFSDPLSLEAQLKSFTKAIKENTN

SEQ ID NO: 37 - DNA, 576 bp, Streptococcus agalactiae
atgaaagtgaaaaataagattttaacgatggtagcacttactgtcttaacatgtgctacttattcatcaatcggttatgctgatacaagtgataagaatactgacacgagtgtcgtgactacgaccttatctgaggagaaaagatcagatgaactagaccagtctagtactggttcttcttctgaaaatgaatcgagttcatcaagtgaaccagaaacaaatccgtcaactaatccacctacaacagaaccatcgcaaccctcacctagtgaagagaacaagcctgatggtagaacgaagacagaaattggcaataataaggatatttctagtggaacaaaagtattaatttcagaagatagtattaagaattttagtaaagcaagtagtgatcaagaagaagtggatcgcgatgaatcatcatcttcaaaagcaaatgatgggaaaaaaggccacagtaagcctaaaaaggaacttcctaaaacaggagatagccactcagatactgtaatagcatctacgggagggattattctgttatcattaagtttttacaataagaaaatgaaactttat

SEQ ID NO: 38 - protein, 192 aa, Streptococcus agalactiae
MKVKNKILTMVALTVLTCATYSSIGYADTSDKNTDTSVVTTTLSEEKRSDELDQSSTGSSSENESSSSSEPETNPSTNPPTTEPSQPSPSEENKPDGRTKTEIGNNKDISSGTKVLISEDSIKNFSKASSDQEEVDRDESSSSKANDGKKGHSKPKKELPKTGDSHSDTVIASTGGIILLSLSFYNKKMKLY

SEQ ID NO: 39 - DNA, 924 bp, Streptococcus agalactiae
atgaaaaggatacggaaaagccttatttttgttctcggagtagttaccctaatttgcttatgtgcttgtactaaacaaagccagcaaaaaaatggcttgtcagtagtgactagcttttatccagtatattccattacaaaagcagtttctggtgatttgaatgatattaaaatgattcgatcacagtcaggtattcatggttttgaaccctcatcaagtgatgttgctgccatttatgatgctgatctatttctttatcattcgcacacactagaagcttgggcgagacgtttggaacctagtttgcatcactctaaagtatctgtaattgaagcttcaaaaggtatgactttggataaagttcatggcttagaagatgtagaggcagaaaaaggagtagatgagtcaaccttgtatgaccctcacacttggaatgaccctgtaaaagtatctgaggaagcacaactcatcgctacacaattagctaaaaaggatcctaaaaacgctaaggtttatcaaaaaaatgctgatcaatttagtgacaaggcaatggctattgcagagaagtataagccaaaatttaaagctgcaaagtctaaatactttgtgacttcacatacagcattctcatacttagctaagcgatacggattgactcagttaggtattgcaggtgtctcaaccgagcaagaacctagtgctaaaaaattagccgaaattcaggagtttgtgaaaacatataaggttaagactatttttgttgaagaaggagtctcacctaaattagctcaagcagtagcttcagctactcgagttaaaattgcaagtttaagtcctttaraagcagttcccaaaaacaataaagattacttagaaaatttggaaactaatcttaaggtacttgtcaaatcgttaaatcaatag

SEQ ID NO: 40 - protein, 307 aa, Streptococcus agalactiae
  VARIANT (1)...(307): Xaa = Any Amino Acid
MKRIRKSLIFVLGVVTLICLCACTKQSQQKNGLSVVTSFYPVYSITKAVSGDLNDIKMIRSQSGIHGFEPSSSDVAAIYDADLFLYHSHTLEAWARRLEPSLHHSKVSVIEASKGMTLDKVHGLEDVEAEKGVDESTLYDPHTWNDPVKVSEEAQLIATQLAKKDPKNAKVYQKNADQFSDKAMAIAEKYKPKFKAAKSKYFVTSHTAFSYLAKRYGLTQLGIAGVSTEQEPSAKKLAEIQEFVKTYKVKTIFVEEGVSPKLAQAVASATRVKIASLSPLXAVPKNNKDYLENLETNLKVLVKSLNQ

SEQ ID NO: 41 - DNA, 1134 bp, Streptococcus agalactiae
atgcctaagaagaaatcagataccccagaaaaagaagaagttgtcttaacggaatggcaaaagcgtaaccttgaatttttaaaaaaacgcaaagaagatgaagaagaacaaaaacgtattaacgaaaaattacgcttagataaaagaagtaaattaaatatttcttctcctgaagaacctcaaaatactactaaaattaagaagcttcattttccaaagatttcaagacctaagattgaaaagaaacagaaaaaagaaaaaatagtcaacagcttagccaaaactaatcgcattagaactgcacctatatttgtagtagcattcctagtcattttagtttccgttttcctactaactccttttagtaagcaaaaaacaataacagttagtggaaatcagcatacacctgatgatattttgatagagaaaacgaatattcaaaaaaacgattatttcttttctttaatttttaaacataaagctattgaacaacgtttagctgcagaagatgtatgggtaaaaacagctcagatgacttatcaatttcccaataagtttcatattcaagttcaagaaaataagattattgcatatgcacatacaaagcaaggatatcaacctgtcttggaaactggaaaaaaggctgatcctgtaaatagttcagagctaccaaagcacttcttaacaattaaccttgataaggaagatagtattaagctattaattaaagatttaaaggctttagaccctgatttaataagtgagattcaggtgataagtttagctgattctaaaacgacacctgacctcctgctgttagatatgcacgatggaaatagtattagaataccattatctaaatttaaagaaagacttcctttttacaaacaaattaagaagaaccttaaggaaccttctattgttgatatggaagtgggagtttacacaacaacaaataccattgaatcaacccctgttaaagcagaagatacaaaaaataaatcaactgataaaacacaaacacaaaatggtcaggttgcggaaaatagtcaaggacaaacaaataactcaaatactaatcaacaaggacaacagatagcaacagagcaggcacctaaccctcaaaatgttaat

SEQ ID NO: 42 - protein, 378 aa, Streptococcus agalactiae
MPKKKSDTPEKEEVVLTEWQKRNLEFLKKRKEDEEEQKRINEKLRLDKRSKLNISSPEEPQNTTKIKKLHFPKISRPKIEKKQKKEKIVNSLAKTNRIRTAPIFVVAFLVILVSVFLLTPFSKQKTITVSGNQHTPDDILIEKTNIQKNDYFFSLIFKHKAIEQRLAAEDVWVKTAQMTYQFPNKFHIQVQENKIIAYAHTKQGYQPVLETGKKADPVNSSELPKHFLTINLDKEDSIKLLIKDLKALDPDLISEIQVISLADSKTTPDLLLLDMHDGNSIRIPLSKFKERLPFYKQIKKNLKEPSIVDMEVGVYTTTNTIESTPVKAEDTKNKSTDKTQTQNGQVAENSQGQTNNSNTNQQGQQIATEQAPNPQNVN